(19) United States
(12) Patent Application Publication
(10) Pub. No.: US 2024/0318152 A1
(43) Pub. Date: Sep. 26, 2024

(54) P450-BM3 VARIANTS WITH IMPROVED ACTIVITY

(71) Applicant: Codexis, Inc., Redwood City, CA (US)

(72) Inventors: David Entwistle, San Carlos, CA (US); Jason Fell, Martinez, CA (US); Stephan Jenne, Foster City, CA (US); Anders Matthew Knight, Mountain View, CA (US); Simon Ng, San Francisco, CA (US); Ryan D. Reeves, Redwood City, CA (US); Zara Maxine Seibel, San Francisco, CA (US); Jonathan Vroom, South San Francisco, CA (US)

(21) Appl. No.: 18/516,054

(22) Filed: Nov. 21, 2023

Related U.S. Application Data
(60) Provisional application No. 63/491,247, filed on Mar. 20, 2023.

(51) Int. Cl. C12N 9/02

(52) U.S. Cl. CPC C12N 9/0071; C12Y 114/14001

(57) ABSTRACT

The present invention provides improved P450-BM3 variants with improved activity. In some embodiments, the P450-BM3 variants exhibit improved activity on a 1-tert-Butoxycarbonylaminocyclopentanoic acid substrate.

### CROSS-REFERENCE TO RELATED APPLICATIONS

This application claims priority under 35 U.S.C. § 119(e) to U.S. Provisional Application No. 63/491,247, filed Mar. 20, 2023 which is incorporated herein by reference in its entirety.

### FIELD OF THE INVENTION

The present invention provides improved P450-BM3 variants with improved activity. In some embodiments, the P450-BM3 variants exhibit improved activity on a 1-tert-Butoxycarbonylaminocyclopentanoic acid substrate.

### REFERENCE TO SEQUENCE LISTING, TABLE OR COMPUTER PROGRAM

The official copy of the Sequence Listing is submitted concurrently with the specification as an XML file, with a file name of “CX2-233USP2_ST26.xml”, a creation date of Mar. 17, 2023, and a size of 4,511 kilobytes. The Sequence Listing filed is part of the specification and is incorporated in its entirety by reference herein.

### BACKGROUND OF THE INVENTION

The cytochrome P450 monooxygenases (“P450s”) comprise a large group of widely-distributed heme enzymes that are ubiquitous in the natural world. Cytochrome P450-BM3 (“P450-BM3”), obtained from Bacillus megaterium (also now known as Priestia megaterium) catalyzes the NADPH-dependent hydroxylation of long-chain fatty acids, alcohols, and amides, as well as the epoxidation of unsaturated fatty acids (See e.g., Narhi and Fulco, J. Biol. Chem., 261:7160-7169 [1986]; and Capdevila et al., J. Biol. Chem., 271:2263-22671 [1996]). P450-BM3 is unique, in that the reductase (65 kDa) and monooxygenase (55 kDa) domains of the enzyme are fused and produced as a catalytically self-sufficient 120 kDa enzyme. Although these enzymes have been the subject of numerous studies, there remains a need in the art for improved P450s that exhibit high levels of enzymatic activity on various substrates, including 1-tert-Butoxycarbonylaminocyclopentanoic acid.

### SUMMARY OF THE INVENTION

The present invention provides improved P450-BM3 variants with improved activity. In some embodiments, the P450-BM3 variants exhibit improved activity on a 1-tert-Butoxycarbonylaminocyclopentanoic acid substrate. The present disclosure provides a recombinant cytochrome P450-BM3 variant having at least 80% sequence identity to a polypeptide sequence comprising the sequence set forth in SEQ ID NOs: 4, 36, 66, 72, 198, 226, 244, 286, 358, 410, 534, 734, 748, 828, 968, 984, 1160, or 1266. In some further embodiments, the recombinant cytochrome P450-BM3 variants oxidize 1-tert-Butoxycarbonylaminocyclopentanoic acid.

The present invention provides novel biocatalysts and associated methods of use for the synthesis of (tert-Butoxycarbonylamino)-cyclopentanoic acid compounds and related ketone compounds from 1-tert-Butoxycarbonylaminocyclopentanoic acid. The P450-BM3 variants of the present disclosure are engineered variants of a polypeptide (SEQ ID NO: 4), which is an engineered variant of the wild-type enzyme from Bacillus megaterium (SEQ ID NO: 2). These engineered polypeptides are capable of catalyzing the conversion of 1-tert-Butoxycarbonylaminocyclopentanoic acid to (tert-Butoxycarbonylamino)-cyclopentanoic acid products, which are useful in the production of active pharmaceutical ingredients.

The present invention provide engineered cytochrome P450-BM3 variants comprising a polypeptide sequence having at least 85%, 86%, 87%, 88%, 89%, 90%, 91%, 92%, 93%, 94%, 95%, 96%, 97%, 98%, 99% or more sequence identity to SEQ ID NO:4, 36, 66, 72, 198, 226, 244, 286, 358, 410, 534, 734, 748, 828, 968, 984, 1160, or 1266, or a functional fragment thereof, wherein the polypeptide sequence of the engineered cytochrome P450-BM3 variant comprises at least one substitution or substitution set and wherein the amino acid positions of the polypeptide sequence are numbered with reference to SEQ ID NO:4, 36, 66, 72, 198, 226, 244, 286, 358, 410, 534, 734, 748, 828, 968, 984, 1160, or 1266.

The present invention provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 4, comprising at least one substitution or one substitution set at one or more positions selected from 32/83/88, 32/83/88/176, 32/83/88/231/574, 32/83/88/574, 52/83/88, 52/83/88/105, 52/83/88/231, 52/83/88/231/433/574, 52/83/88/433, 52/83/88/433/574, 52/83/88/574, 83/88, 83/88/105, 83/88/111, 83/88/111/433, 83/88/111/574, 83/88/231, 83/88/349, 83/88/433/574, and 83/88/574, wherein the positions are numbered with reference to SEQ ID NO: 4. In some additional embodiments, the engineered polypeptide comprises at least one substitution or one substitution set selected from 32K/83A/88A, 32K/83A/88A/1761, 32K/83A/88A/231R/574T, 32K/83A/88A/574T, 52Y/83A/88A, 52Y/83A/88A/105V, 52Y/83A/88A/231R, 52Y/83A/88A/231R/433D/574T, 52Y/83A/88A/433D, 52Y/83A/88A/433D/574T, 52Y/83A/88A/574T, 83A/88A, 83A/88A/105L, 83A/88A/111Q, 83A/88A/111Q/433D, 83A/88A/111Q/574T, 83A/88A/231R, 83A/88A/349E, 83A/88A/433D/574T, and 83A/88A/574T, wherein the positions are numbered with reference to SEQ ID NO: 4. In some embodiments, the engineered polypeptide comprises at least one substitution or one substitution set selected from R32K/L83A/F88A, R32K/L83A/F88A/V1761, R32K/L83A/F88A/S231R/N574T, R32K/L83A/F88A/N574T, F52Y/L83A/F88A, F52Y/L83A/F88A/G105V, F52Y/L83A/F88A/S231R, F52Y/L83A/F88A/S231R/V433D/N574T, F52Y/L83A/F88A/V433D, F52Y/L83A/F88A/V433D/N574T, F52Y/L83A/F88A/N574T, L83A/F88A, L83A/F88A/G105L, L83A/F88A/R111Q, L83A/F88A/R111Q/V433D, L83A/F88A/R111Q/N574T, L83A/F88A/S231R, L83A/F88A/T349E, L83A/F88A/V433D/N574T, and L83A/F88A/N574T, wherein the positions are numbered with reference to SEQ ID NO: 4.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 36, comprising at least one substitution or one substitution set selected from: 75, 75/374, 75/374/458/726, 75/374/726, 75/458, 75/458/726, 75/726, 111/114, 111/603/604/623/853, 111/623, 374/726, and 726, wherein the positions are numbered with reference to SEQ ID NO: 36. In some embodiments, the engineered polypeptide comprises at least one substitution or one substitution set selected from 75S, 75S/374S, 75S/374S/458L/726L, 75S/374S/726L, 75S/458L, 75S/458L/726L, 75S/726L, 111H/114G, 111H/603F/604G/623Q/853E, 111H/623Q, 374S/726L, and 726L, wherein the positions are numbered with reference to SEQ ID NO: 36. In some further embodiments, the engineered polypeptide comprises at least one substitution or one substitution set selected from A75S, A75S/E374S, A75S/E374S/G458L/Q726L, A75S/E374S/Q726L, A75S/G458L, A75S/G458L/Q726L, A75S/Q726L, R111H/K114G, R111H/E603F/A604G/S623Q/P853E, R111H/S623Q, E374S/Q726L, and Q726L, wherein the positions are numbered with reference to SEQ ID NO: 36.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 66, comprising at least one substitution or one substitution set selected from: 74, 75, 83, 179, 181, 182, 186, 189, 238, 267, 268, 328, 331, 355, 358, 437, and 438, wherein the positions are numbered with reference to SEQ ID NO: 66. In some embodiments, the engineered polypeptide comprises at least one substitution or one substitution set selected from 74G, 75K, 75T, 83V, 179M, 179R, 181V, 182I, 182V, 186R, 189G, 189T, 238L, 267T, 268Q, 328S, 328V, 331F, 331M, 331T, 355V, 358T, 358V, 437G, 437S, and 438R, wherein the positions are numbered with reference to SEQ ID NO: 66. In some further embodiments, the engineered polypeptide comprises at least one substitution or one substitution set selected from Q74G, A75K, A75T, A83V, V179M, V179R, A181V, L182I, L182V, M186R, L189G, L189T, M238L, H267T, E268Q, T328S, T328V, A331F, A331M, A331T, M355V, I358T, I358V, T437G, T437S, and L438R, wherein the positions are numbered with reference to SEQ ID NO: 66.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 72, comprising at least one substitution or one substitution set selected from: 74/75/83/179/189/328/331/437, 74/75/268/328/331/358/437, 74/83/268/328/331/358/437, 74/267/268/328, 75/83/179/189/331, 75/83/179/268/328/331/437, 75/83/189/267/268, 75/83/267/268/355/358/654, 75/83/268/437, 75/268/328/331/358, 83/179/182/437, 83/179/189/331/355, 83/179/328/331, 83/179/355/358/437, 83/182/189/268/328/331/355/358/437, 83/182/189/328/331, 83/182/268/328/355/358/437, 83/189/267/268/358, 83/189/268/328/331, 83/189/268/328/355/358/437, 83/189/328/331/437, 83/267/268/328/331/355/358, 83/268, 83/268/328/331, 83/268/328/331/355/358, 83/268/328/331/358, 83/268/328/331/358/437, 83/268/328/331/437, 83/268/331, 83/331, 83/331/437, 83/358/437, 179/182/268, 179/189/331/437, 179/328/331, 179/331/358, 189/267/268/437, 189/268/328, 189/268/328/331/358/437, 189/268/358, 267/268/328, 267/268/328/331/355/358, 267/268/331/355/358/437, 268, 268/328/331, 268/328/355/358, 268/331/355/358, 268/355/358/437, 268/358, and 328/331/358, wherein the positions are numbered with reference to SEQ ID NO: 72. In some embodiments, the engineered polypeptide comprises at least one substitution or one substitution set selected from 74G/75T/83V/179R/189G/328V/331T/437G, 74G/75T/268Q/328V/331T/358V/437S, 74G/83V/268Q/328S/331M/358V/437G, 74G/267T/268Q/328V, 75T/83V/179R/189G/331M, 75T/83V/179R/268Q/328V/331M/437G, 75T/83V/189G/267T/268Q, 75T/83V/267T/268Q/355V/358V/654L, 75T/83V/268Q/437G, 75T/268Q/328V/331T/358T, 83V/179R/182V/437S, 83V/179R/189G/331M/355V, 83V/179R/328V/331M, 83V/179R/355V/358V/437G, 83V/182I/189G/268Q/328V/331M/355V/358V/437G, 83V/182I/189G/328V/331M, 83V/182I/268Q/328V/355V/358V/437S, 83V/189G/267T/268Q/358T, 83V/189G/268Q/328V/331M, 83V/189G/268Q/328V/355V/358V/437G, 83V/189G/328V/331M/437G, 83V/267T/268Q/328V/331M/355V/358T, 83V/268Q, 83V/268Q/328S/331M/355V/358V, 83V/268Q/328S/331M/358T/437G, 83V/268Q/328S/331T/437G, 83V/268Q/328V/331M, 83V/268Q/328V/331M/358V, 83V/268Q/331T, 83V/331M, 83V/331T/437S, 83V/358V/437S, 179R/182I/268Q, 179R/189G/331M/437S, 179R/328V/331M, 179R/331M/358V, 189G/267T/268Q/437S, 189G/268Q/328V, 189G/268Q/328V/331T/358V/437S, 189G/268Q/358V, 267T/268Q/328V, 267T/268Q/328V/331M/355V/358T, 267T/268Q/331T/355V/358T/437S, 268Q, 268Q/328V/331M, 268Q/328V/355V/358T, 268Q/331M/355V/358V, 268Q/355V/358V/437G, 268Q/358V, and 328V/331M/358T, wherein the positions are numbered with reference to SEQ ID NO: 72. In some further embodiments, the engineered polypeptide comprises at least one substitution or one substitution set selected from Q74G/A75T/A83V/V179R/L189G/T328V/A331T/T437G, Q74G/A75T/E268Q/T328V/A331T/I358V/T437S, Q74G/A83V/E268Q/T328S/A331M/I358V/T437G, Q74G/H267T/E268Q/T328V, A75T/A83V/V179R/L189G/A331M, A75T/A83V/V179R/E268Q/T328V/A331M/T437G, A75T/A83V/L189G/H267T/E268Q, A75T/A83V/H267T/E268Q/M355V/I358V/M654L, A75T/A83V/E268Q/T437G, A75T/E268Q/T328V/A331T/I358T, A83V/V179R/L182V/T437S, A83V/V179R/L189G/A331M/M355V, A83V/V179R/T328V/A331M, A83V/V179R/M355V/I358V/T437G, A83V/L182I/L189G/E268Q/T328V/A331M/M355V/I358V/T437G, A83V/L182I/L189G/T328V/A331M, A83V/L182I/E268Q/T328V/M355V/I358V/T437S, A83V/L189G/H267T/E268Q/I358T, A83V/L189G/E268Q/T328V/A331M, A83V/L189G/E268Q/T328V/M355V/I358V/T437G, A83V/L189G/T328V/A331M/T437G, A83V/H267T/E268Q/T328V/A331M/M355V/I358T, A83V/E268Q, A83V/E268Q/T328S/A331M/M355V/I358V, A83V/E268Q/T328S/A331M/I358T/T437G, A83V/E268Q/T328S/A331T/T437G, A83V/E268Q/T328V/A331M, A83V/E268Q/T328V/A331M/I358V, A83V/E268Q/A331T, A83V/A331M, A83V/A331T/T437S, A83V/I358V/T437S, V179R/L182I/E268Q, V179R/L189G/A331M/T437S, V179R/T328V/A331M, V179R/A331M/I358V, L189G/H267T/E268Q/T437S, L189G/E268Q/T328V, L189G/E268Q/T328V/A331T/I358V/T437S, L189G/E268Q/I358V, H267T/E268Q/T328V, H267T/E268Q/T328V/A331M/M355V/I358T, H267T/E268Q/A331T/M355V/I358T/T437S, E268Q, E268Q/T328V/A331M, E268Q/T328V/M355V/I358T, E268Q/A331M/M355V/I358V, E268Q/M355V/I358V/T437G, E268Q/I358V, and T328V/A331M/I358T, wherein the positions are numbered with reference to SEQ ID NO: 72.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 198, comprising at least one substitution or one substitution set selected from: 79, 213, and 257, wherein the positions are numbered with reference to SEQ ID NO: 198. In some embodiments, the engineered polypeptide comprises at least one substitution or one substitution set selected from 79I, 213V, and 257Q, wherein the positions are numbered with reference to SEQ ID NO: 198. In some further embodiments, the engineered polypeptide comprises at least one substitution or one substitution set selected from A79I, M213V, and Y257Q, wherein the positions are numbered with reference to SEQ ID NO: 198.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 226, comprising at least one substitution or one substitution set selected from: 315, 320, 385, 388, 391, 398, 405, 493, 497, 502, 503, 504, 541, 542, 547, 573, 576 and 577, wherein the positions are numbered with reference to SEQ ID NO: 226.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 244, comprising at least one substitution or one substitution set selected from: 75/178/213/315, 75/331/576/726, 178, 178/179/213/437/497/573/576, 178/179/573/726, 178/179/576, 178/213/573, 178/213/726, 178/437, 178/497/726, 178/576, 178/726, 179/358, 179/726, 331, 331/358/391/437, 331/497, 331/573/576, 497/573, 573, 682, 685, 699, 701, 704, 707, 708, 726, 756, 759, 794, 796, 797, 848, 851, 862, 888, 889, 999, 1003, and 1048, wherein the positions are numbered with reference to SEQ ID NO: 244. In some embodiments, the engineered polypeptide comprises at least one substitution or one substitution set selected from 75S/178V/213V/315T, 75S/331T/576V/726L, 178V, 178V/179R/213V/4375/497A/573S/576V, 178V/179R/573S/726L, 178V/179R/576V, 178V/213V/573S, 178V/213V/726L, 178V/437S, 178V/497A/726L, 178V/576V, 178V/726L, 179R/358T, 179R/726L, 331T, 331T/358T/391Y/437S, 331T/497A, 331T/573S/576V, 497A/573S, 573S, 682K, 685F, 699R, 701V, 704L, 707Y, 708C, 726L, 756Q, 759T, 794C, 796H, 796N, 797H, 848T, 851G, 851V, 862L, 888Y, 889Q, 999A, 1003W, and 1048L, wherein the positions are numbered with reference to SEQ ID NO: 244. In some further embodiments, the engineered polypeptide comprises at least one substitution or one substitution set selected from A75S/M178V/M213V/V315T, A75S/M331T/A576V/Q726L, M178V, M178V/V179R/M213V/T437S/T497A/K573S/A576V, M178V/V179R/K573S/Q726L, M178V/V179R/A576V, M178V/M213V/K573S, M178V/M213V/Q726L, M178V/T437S, M178V/T497A/Q726L, M178V/A576V, M178V/Q726L, V179R/V358T, V179R/Q726L, M331T, M331T/V358T/F391Y/T437S, M331T/T497A, M331T/K573S/A576V, T497A/K573S, K573S, T682K, L685F, D699R, L701V, 1704L, N707Y, Y708C, Q726L, L756Q, P759T, V794C, A796H, A796N, K797H, S848T, S851G, S851V, K862L, E888Y, F889Q, 1999A, G1003W, and A1048L, wherein the positions are numbered with reference to SEQ ID NO: 244.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 286, comprising at least one substitution or one substitution set selected from: 75/331/701/726/851/1048, 75/331/726/999, 75/726/796/851/999, 75/1048, 87, 88/522, 89, 234, 269, 328, 330, 331, 398, 405, 408, and 411, wherein the positions are numbered with reference to SEQ ID NO: 286. In some embodiments, the engineered polypeptide comprises at least one substitution or one substitution set selected from 75S/331T/701V/726L/851V/1048L, 75S/331T/726L/999A, 75S/726L/796N/851V/999A, 75S/1048L, 871, 87V, 88S/522R, 89S, 234F, 269G, 269P, 328T, 330G, 331S, 398R, 405A, 405S, 408R, and 411G, wherein the positions are numbered with reference to SEQ ID NO: 286. In some further embodiments, the engineered polypeptide comprises at least one substitution or one substitution set selected from A75S/M331T/L701V/Q726L/S851V/A1048L, A75S/M331T/Q726L/1999A, A75S/Q726L/A796N/S851V/I999A, A75S/A1048L, L87I, L87V, A88S/G522R, T89S, L234F, T269G, T269P, V328T, P330G, M331S, Q398R, Q405A, Q405S, L408R, and A411G, wherein the positions are numbered with reference to SEQ ID NO: 286.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 358, comprising at least one substitution or one substitution set selected from: 75/269/707, 269, 269/522/707, 269/522/707/1048, 522, 522/726, 522/1048, and 726/1048, wherein the positions are numbered with reference to SEQ ID NO: 358. In some embodiments, the engineered polypeptide comprises at least one substitution or one substitution set selected from 75S/269P/707Y, 269P, 269P/522G/707Y, 269P/522G/707Y/1048L, 522G, 522G/726L, 522G/1048L, and 726L/1048L, wherein the positions are numbered with reference to SEQ ID NO: 358. In some further embodiments, the engineered polypeptide comprises at least one substitution or one substitution set selected from A75S/T269P/N707Y, T269P, T269P/R522G/N707Y, T269P/R522G/N707Y/A1048L, R522G, R522G/Q726L, R522G/A1048L, and Q726L/A1048L, wherein the positions are numbered with reference to SEQ ID NO: 358.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 358, comprising at least one substitution or one substitution set selected from: 77, 170, 286, 289, 462, 547, 557, 630, 646, 651, 672, 676, 692, 775, 786, 787, 788, 814, 841, 876, 877, 888, 893, 896, 924, 941, 955, 969, 973, 982, 989, 993, and 1038, wherein the positions are numbered with reference to SEQ ID NO: 358. In some embodiments, the engineered polypeptide comprises at least one substitution or one substitution set selected from 77L, 170S, 170T, 286R, 289A, 462W, 547R, 557E, 557P, 630G, 646F, 651S, 672G, 676K, 676R, 692G, 692V, 775K, 786G, 786R, 786S, 786V, 787R, 788P, 8141, 814S, 841R, 876G, 877V, 888G, 893G, 896L, 924A, 924G, 924N, 924P, 941R, 955G, 969E, 969G, 973P, 982G, 989G, 989L, 993S, and 1038Q, wherein the positions are numbered with reference to SEQ ID NO: 358. In some further embodiments, the engineered polypeptide comprises at least one substitution or one substitution set selected from K77L, Q170S, Q170T, H286R, Q289A, P462W, Q547R, A557E, A557P, N630G, Q646F, A651S, E672G, P676K, P676R, E692G, E692V, P775K, E786G, E786R, E786S, E786V, K787R, Q788P, K8141, K814S, K841R, D876G, T877V, E888G, K893G, E896L, Q924A, Q924G, Q924N, Q924P, H941R, S955G, P969E, P969G, K973P, Q982G, E989G, E989L, Q993S, and E1038Q, wherein the positions are numbered with reference to SEQ ID NO: 358.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 410, comprising at least one substitution or one substitution set selected from: 158/170, 158/170/410/462/630/672/726/786/788/814/924, 158/170/410/462/726/786/814, 158/170/410/924, 158/170/630/726/786/788/814, 158/410, 158/410/462/557/969, 158/410/557/786/787, 158/410/814/924, 158/410/862, 158/462/557/630/924, 158/462/630/786/788/969, 158/557, 158/557/630/814, 158/557/726/786/788/862, 158/557/786, 158/557/786/787/788, 158/557/814/924, 158/630/786/814, 158/726, 158/786/787/788, 158/814/924, 170/410/557/786/787/814/924/989, 385, 410/462/557/630/786/787/969, 462/557/726/786/787/924, 469, 523, 550, 553, 556, 574, 613, 640, 645, 650, 652, 717, 773, 779, 795, 838, 871, 923, and 926, wherein the positions are numbered with reference to SEQ ID NO: 410. In some embodiments, the engineered polypeptide comprises at least one substitution or one substitution set selected from 158L/170T, 158L/170T/410S/462W/630G/672G/726L/786R/788P/814S/924G, 158L/170T/410S/462W/726L/786G/814S, 158L/170T/410S/924G, 158L/170T/630G/726L/786G/788P/814S, 158L/410S, 158L/410S/462W/557E/969G, 158L/410S/557P/786G/787R, 158L/410S/814S/924G, 158L/410S/862L, 158L/462W/557P/630G/924G, 158L/462W/630G/786R/788P/969G, 158L/557P, 158L/557P/630G/814S, 158L/557P/726L/786G/788P/862L, 158L/557P/786R/787R/788P, 158L/557P/786V, 158L/557P/814S/924G, 158L/630G/786S/814S, 158L/726L, 158L/786S/787R/788P, 158L/814S/924G, 170T/410S/557E/786V/787R/814S/924G/989G, 385R, 385T, 410S/462W/557P/630G/786V/787R/969G, 462W/557P/726L/786G/787R/924G, 469R, 523R, 550T, 553A, 553G, 553K, 553S, 556G, 574H, 574S, 613A, 640C, 640G, 640V, 640W, 645V, 650P, 652S, 717C, 717Q, 773A, 773G, 779G, 795A, 838W, 871M, 871N, 923A, 923L, 926K, and 926R, wherein the positions are numbered with reference to SEQ ID NO: 410. In some further embodiments, the engineered polypeptide comprises at least one substitution or one substitution set selected from G158L/Q170T, G158L/Q170T/E410S/P462W/N630G/E672G/Q726L/E786R/Q788P/K814S/Q924G, G158L/Q170T/E410S/P462W/Q726L/E786G/K814S, G158L/Q170T/E410S/Q924G, G158L/Q170T/N630G/Q726L/E786G/Q788P/K814S, G158L/E410S, G158L/E410S/P462W/A557E/P969G, G158L/E410S/A557P/E786G/K787R, G158L/E410S/K814S/Q924G, G158L/E410S/K862L, G158L/P462W/A557P/N630G/Q924G, G158L/P462W/N630G/E786R/Q788P/P969G, G158L/A557P, G158L/A557P/N630G/K814S, G158L/A557P/Q726L/E786G/Q788P/K862L, G158L/A557P/E786R/K787R/Q788P, G158L/A557P/E786V, G158L/A557P/K814S/Q924G, G158L/N630G/E786S/K814S, G158L/Q726L, G158L/E786S/K787R/Q788P, G158L/K814S/Q924G, Q170T/E410S/A557E/E786V/K787R/K814S/Q924G/E989G, A385R, A385T, E410S/P462W/A557P/N630G/E786V/K787R/P969G, P462W/A557P/Q726L/E786G/K787R/Q924G, K469R, N523R, D550T, D553A, D553G, D553K, D553S, S556G, N574H, N574S, T613A, K640C, K640G, K640V, K640W, L645V, S650P, A652S, A717C, A717Q, V773A, V773G, V779G, L795A, V838W, E871M, E871N, E923A, E923L, Q926K, and Q926R, wherein the positions are numbered with reference to SEQ ID NO: 410.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 410, comprising at least one substitution or one substitution set selected from: 51/851, 460, 466, 474, 597, 600, 635, 638, 655, 663, 664, 677, 694, 696, 713, 771, 783, 789, 806, 807, 840, 842, 851, 857, 860, 878, 894, 942, 947, 960, 978, 992, 1008, 1012, 1024, and 1025, wherein the positions are numbered with reference to SEQ ID NO: 410. In some embodiments, the engineered polypeptide comprises at least one substitution or one substitution set selected from 51H/851Q, 460R, 466C, 474F, 474P, 597R, 597V, 600K, 635S, 638E, 638W, 655A, 655L, 655T, 663G, 664L, 677H, 677N, 694P, 696C, 713M, 771I, 783E, 783K, 783S, 789G, 806N, 807R, 807T, 840L, 842G, 851L, 857F, 860R, 878S, 894V, 942K, 947W, 960A, 960G, 960K, 978S, 992F, 992G, 1008C, 1012G, 1012M, 1024I, 1024L, 1025G, 1025T, and 1025W, wherein the positions are numbered with reference to SEQ ID NO: 410. In some further embodiments, the engineered polypeptide comprises at least one substitution or one substitution set selected from R51H/S851Q, P460R, Q466C, K474F, K474P, N597R, N597V, D600K, N635S, D638E, D638W, P655A, P655L, P655T, F663G, S664L, G677H, G677N, S694P, Q696C, N713M, K771I, A783E, A783K, A783S, A789G, E806N, K807R, K807T, E840L, Q842G, S851L, G857F, E860R, I878S, D894V, E942K, Q947W, T960A, T960G, T960K, H978S, D992F, D992G, P1008C, A1012G, A1012M, V1024I, V1024L, S1025G, S1025T, and S1025W, wherein the positions are numbered with reference to SEQ ID NO: 410.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 544, comprising at least one substitution or one substitution set selected from: 77/179/286/410/788/888, 77/410/676/788/924, 77/557/707/888, 286/410/651/676, 286/410/707/788, 286/410/888, 286/692/786/788, 410, 410/557/676/788/888/924/993, 410/557/692/788, and 410/646/651/788, wherein the positions are numbered with reference to SEQ ID NO: 544. In some embodiments, the engineered polypeptide comprises at least one substitution or one substitution set selected from 77L/179L/286R/410S/788Q/888G, 77L/410S/676K/788Q/924N, 77L/557P/707Y/888G, 286R/410S/651S/676K, 286R/410S/707Y/788Q, 286R/410S/888G, 286R/692V/786R/788Q, 410S, 410S/557P/676K/788Q/888G/924N/993S, 410S/557P/692V/788Q, and 410S/646F/651S/788Q, wherein the positions are numbered with reference to SEQ ID NO: 544. In some further embodiments, the engineered polypeptide comprises at least one substitution or one substitution set selected from K77L/V179L/H286R/E410S/P788Q/E888G, K77L/E410S/P676K/P788Q/Q924N, K77L/A557P/N707Y/E888G, H286R/E410S/A651S/P676K, H286R/E410S/N707Y/P788Q, H286R/E410S/E888G, H286R/E692V/G786R/P788Q, E410S, E410S/A557P/P676K/P788Q/E888G/Q924N/Q993S, E410S/A557P/E692V/P788Q, and E410S/Q646F/A651S/P788Q, wherein the positions are numbered with reference to SEQ ID NO: 544.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 734, comprising at least one substitution or one substitution set selected from: 24, 53, 75, 78, 82, 88, 150, 180, 183, 257, 270, 410/497/557/576/814, 410/497/573/576, 410/497/814, 410/557/924, 437, and 497/557, wherein the positions are numbered with reference to SEQ ID NO: 734. In some embodiments, the engineered polypeptide comprises at least one substitution or one substitution set selected from 24R, 24S, 53V, 75G, 78E, 82I, 88G, 88T, 88V, 150G, 180G, 183S, 257G, 257H, 257N, 257W, 270I, 270V, 410E/497A/557P/576V/814K, 410E/497A/573S/576V, 410E/497A/814K, 410E/557P/924G, 437N, 437V, and 497A/557P, wherein the positions are numbered with reference to SEQ ID NO: 734. In some further embodiments, the engineered polypeptide comprises at least one substitution or one substitution set selected from D24R, D24S, L53V, A75G, F78E, F82I, S88G, S88T, S88V, T150G, R180G, D183S, Q257G, Q257H, Q257N, Q257W, T270I, T270V, S410E/T497A/A557P/A576V/S814K, S410E/T497A/K573S/A576V, S410E/T497A/S814K, S410E/A557P/Q924G, S437N, S437V, and T497A/A557P, wherein the positions are numbered with reference to SEQ ID NO: 734.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 748, comprising at least one substitution or one substitution set selected from: 75, 75/82/180/257/437, 75/82/257/268, 75/82/257/556/640, 75/82/257/773, 75/82/437, 75/180/183/257/268/270/385/437/556/613/652/923, 75/180/257/268/270/437/556/574/652, 75/180/574/795, 75/257/613, 75/257/640, 75/556/773, 75/574, 75/613, 82/613, 180/437/773/795, and 257/613/773/795, wherein the positions are numbered with reference to SEQ ID NO: 748. In some embodiments, the engineered polypeptide comprises at least one substitution or one substitution set selected from 75G, 75G/82I/180G/257G/437N, 75G/82I/257G/268T, 75G/82I/257H/773A, 75G/82I/257N/556G/640G, 75G/82I/437N, 75G/180G/183S/257N/268T/270I/385R/437N/556G/613A/652S/923A, 75G/180G/257N/268T/270I/437V/556G/574S/652S, 75G/180G/574S/795A, 75G/257N/613A, 75G/257N/640W, 75G/556G/773A, 75G/574S, 75G/613A, 82I/613A, 180G/437N/773A/795A, and 257H/613A/773A/795A, wherein the positions are numbered with reference to SEQ ID NO: 748. In some further embodiments, the engineered polypeptide comprises at least one substitution or one substitution set selected from A75G, A75G/F82I/R180G/Q257G/S437N, A75G/F82I/Q257G/Q268T, A75G/F82I/Q257H/V773A, A75G/F82I/Q257N/S556G/K640G, A75G/F82I/S437N, A75G/R180G/D183S/Q257N/Q268T/T270I/A385R/S437N/S556G/T613A/A652S/E923A, A75G/R180G/Q257N/Q268T/T270I/S437V/S556G/N574S/A652S, A75G/R180G/N574S/L795A, A75G/Q257N/T613A, A75G/Q257N/K640W, A75G/S556G/V773A, A75G/N574S, A75G/T613A, F82I/T613A, R180G/S437N/V773A/L795A, and Q257H/T613A/V773A/L795A, wherein the positions are numbered with reference to SEQ ID NO: 748.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 748, comprising at least one substitution or one substitution set selected from: 45, 102, 106, 110, 111, 114, 127, 191, 193, 194, 196, 196/853, 198, 202, 203, 206, 210, 226, 232, 236, 237, 244, 245, 248, 254, 256, and 347, wherein the positions are numbered with reference to SEQ ID NO: 748. In some embodiments, the engineered polypeptide comprises at least one substitution or one substitution set selected from 45E, 102A, 102L, 106V, 110F, 110L, 110V, 111L, 111R, 111W, 114H, 114M, 114Q, 114R, 127M, 191S, 193K, 194T, 196Q/853L, 196W, 198S, 202Q, 203G, 206R, 210R, 210T, 226N, 232R, 236G, 236S, 237N, 237R, 244G, 244R, 244Y, 245A, 245G, 245L, 245M, 245N, 245Q, 245R, 245S, 245T, 245V, 245W, 248M, 248R, 254L, 256K, 347E, 347K, and 347S, wherein the positions are numbered with reference to SEQ ID NO: 748. In some further embodiments, the engineered polypeptide comprises at least one substitution or one substitution set selected from A45E, N102A, N102L, P106V, Q110F, Q110L, Q110V, H111L, H111R, H111W, G114H, G114M, G114Q, G114R, L127M, R191S, N193K, P194T, D196Q/P853L, D196W, A198S, N202Q, K203G, C206R, I210R, I210T, A226N, H232R, T236G, T236S, Q237N, Q237R, P244G, P244R, P244Y, E245A, E245G, E245L, E245M, E245N, E245Q, E245R, E245S, E245T, E245V, E245W, E248M, E248R, N254L, R256K, P347E, P347K, and P347S, wherein the positions are numbered with reference to SEQ ID NO: 748.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 828, comprising at least one substitution or one substitution set selected from: 22/75/717/720/779/1004, 22/550/826, 22/616/717/1004, 22/717/795/799/826, 550/616/717/779, 550/640/717, 550/717/795/799/800, 616/717/720/799, 640, 717, 717/720/779/1004, 717/779/799/800/1004, 717/1004, 720/779, 779/1004, 800/1004, and 826/1004, wherein the positions are numbered with reference to SEQ ID NO: 828. In some embodiments, the engineered polypeptide comprises at least one substitution or one substitution set selected from 22D/75A/717C/720L/779F/1004I, 22D/550T/826G, 22D/616L/717I/1004I, 22D/717Q/795V/799I/826G, 550T/616L/717I/779F, 550T/640D/717I, 550T/717C/795V/799I/800Y, 616L/717G/720L/799I, 640V, 717C, 717G/720L/779F/1004I, 717G/1004I, 717Q/779F/799I/800Y/1004I, 717Q/1004I, 720L/779F, 779F/1004I, 800Y/1004I, and 826G/1004I, wherein the positions are numbered with reference to SEQ ID NO: 828. In some further embodiments, the engineered polypeptide comprises at least one substitution or one substitution set selected from N22D/G75A/A717C/G720L/V779F/S1004I, N22D/D550T/R826G, N22D/E616L/A717I/S1004I, N22D/A717Q/L795V/L799I/R826G, D550T/E616L/A717I/V779F, D550T/K640D/A717I, D550T/A717C/L795V/L799I/T800Y, E616L/A717G/G720L/L799I, K640V, A717C, A717G/G720L/V779F/S1004I, A717G/S1004I, A717Q/V779F/L799I/T800Y/S1004I, A717Q/S1004I, G720L/V779F, V779F/S1004I, T800Y/S1004I, and R826G/S1004I, wherein the positions are numbered with reference to SEQ ID NO: 828.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 968, comprising at least one substitution or one substitution set selected from: 3, 118/446, 132, 230, 285, 290, 292, 293, 295, 296, 300, 303, 305, 307, 366, 371, 372, 381, 382, 415, 417, 418, 424, 427, 432, 433, 446, 447, 455, 463, 465, 468, 473, 473/790, 478, 480, 481, 506, 600/635/713/771/1025, 600/840/960, 635/636/793/840, 635/638/793/823/960, 635/713/1025, 635/771/894, 636/638/793/851, 638/663/793/840, 713, 771, 786/840/960, 793/840, 807, 840/960, 851/1024/1025, 851/1025, 960, and 1025, wherein the positions are numbered with reference to SEQ ID NO: 968. In some embodiments, the engineered polypeptide comprises at least one substitution or one substitution set selected from 3D, 3W, 118T/446L, 132M, 132V, 230D, 230V, 285G, 285S, 290S, 292S, 293R, 293T, 295S, 296G, 296Q, 300P, 303Q, 303W, 305G, 305N, 305R, 307R, 366I, 366S, 371K, 372S, 381F, 382M, 382T, 415V, 417V, 418L, 418V, 424V, 424Y, 427T, 432V, 433D, 433K, 433L, 433R, 446S, 447L, 455R, 463A, 463V, 465Q, 468F, 473L/790H, 473R, 478S, 480W, 481L, 506D, 506V, 600K/635S/713M/771I/1025G, 600K/840L/960L, 635S/636G/793T/840L, 635S/638G/793T/823A/960L, 635S/713M/1025T, 635S/771I/894V, 636M/638G/793T/851L, 638G/663R/793T/8405, 713M, 771I, 786A/8405/960L, 793T/840L, 807R, 840L/960L, 851L/1024I/1025T, 851L/1025T, 960L, 1025T, and 1025W, wherein the positions are numbered with reference to SEQ ID NO: 968. In some further embodiments, the engineered polypeptide comprises at least one substitution or one substitution set selected from I3D, I3W, A118T/V446L, E132M, E132V, Q230D, Q230V, P285G, P285S, K290S, A292S, E293R, E293T, A295S, A296G, A296Q, V300P, V303Q, V303W, S305G, S305N, S305R, K307R, T366I, T366S, D371K, V372S, E381F, N382M, N382T, L415V, M417V, M418L, M418V, F424V, F424Y, H427T, L432V, V433D, V433K, V433L, V433R, V446S, V447L, P455R, S463A, S463V, E465Q, A468F, K473L/Y790H, K473R, A478S, N480W, T481L, M506D, M506V, D600K/N635S/N713M/K771I/S1025G, D600K/E840L/T960L, N635S/S636G/Q793T/E840L, N635S/D638G/Q793T/P823A/T960L, N635S/N713M/S1025T, N635S/K771I/D894V, S636M/D638G/Q793T/S851L, D638G/F663R/Q793T/E840S, N713M, K771I, G786A/E840S/T960L, Q793T/E840L, K807R, E840L/T960L, S851L/V1024I/S1025T, S851L/S1025T, T960L, 51025T, and S1025W, wherein the positions are numbered with reference to SEQ ID NO: 968.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 984, comprising at least one substitution or one substitution set selected from: 45, 45/111/226/347, 45/853, 110/114, 111, 111/127/226/244, 111/194/244/347/853, 111/194/347, 111/194/347/853, 111/210, 111/210/347/853, 111/226, 111/226/244/347, 111/226/853/969, 111/244, 111/244/853, 111/347/853, 111/853, 114, 114/245, 127/210/244, 127/210/244/853, 127/210/347/853, 127/244, 127/347, 194/853, 210/853, 226/236/244/347/853, 236/244, 237, 237/245, 244/853, and 245, wherein the positions are numbered with reference to SEQ ID NO: 984. In some embodiments, the engineered polypeptide comprises at least one substitution or one substitution set selected from 45E, 45E/111R/226N/347S, 45E/853L, 110L/114Q, 11 IL, 111L/127M/226N/244Y, 111L/210T, 111L/244R, 111L/244Y/853L, 111L/853L, 111R/127M/226N/244Y, 111R/194T/244Y/347S/853L, 111R/194T/347S, 111R/194T/347S/853L, 111R/210T/347S/853L, 111R/226N, 111R/226N/244Y/347S, 111R/347E/853L, 111R/853L, 111W, 111W/226N/853L/969S, 114Q, 114Q/245V, 127M/210T/244Y, 127M/210T/244Y/853L, 127M/210T/347E/853L, 127M/244Y, 127M/347E, 194T/853L, 210T/853L, 226N/236G/244Y/347S/853L, 236G/244Y, 237R, 237R/245V, 244Y/853L, 245S, and 245V, wherein the positions are numbered with reference to SEQ ID NO: 984. In some further embodiments, the engineered polypeptide comprises at least one substitution or one substitution set selected from A45E, A45E/H111R/A226N/P347S, A45E/P853L, Q110L/G114Q, H111L, H111 L/L127M/A226N/P244Y, H111L/I210T, H111 L/P244R, H111 L/P244Y/P853L, H111 L/P853L, H111R/L127M/A226N/P244Y, H111R/P194T/P244Y/P347S/P853L, H111R/P194T/P347S, H111R/P194T/P347S/P853L, H111R/1210T/P347S/P853L, H11R/A226N, H111R/A226N/P244Y/P347S, H11R/P347E/P853L, H111R/P853L, H11W, H111W/A226N/P853L/P969S, G114Q, G114Q/E245V, L127M/I210T/P244Y, L127M/1210T/P244Y/P853L, L127M/1210T/P347E/P853L, L127M/P244Y, L127M/P347E, P194T/P853L, 1210T/P853L, A226N/T236G/P244Y/P347S/P853L, T236G/P244Y, Q237R, Q237R/E245V, P244Y/P853L, E245S, and E245V, wherein the positions are numbered with reference to SEQ ID NO: 984.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 984, comprising at least one substitution or one substitution set selected from: 518, 518/652, 519, 562, 563, 584/724, 586, 616, 618, 619, 621, 623, 628, 640, 653, and 666, wherein the positions are numbered with reference to SEQ ID NO: 984. In some embodiments, the engineered polypeptide comprises at least one substitution or one substitution set selected from 518G, 518N/652S, 518S, 518T, 519L, 562P, 563L, 563S, 584G/724P, 586V, 616V, 618G, 618L, 619G, 619R, 621T, 623P, 628S, 640L, 653A, 653R, 653T, and 666R, wherein the positions are numbered with reference to SEQ ID NO: 984. In some further embodiments, the engineered polypeptide comprises at least one substitution or one substitution set selected from D518G, D518N/A652S, D518S, D518T, S519L, G562P, V563L, V563S, A584G/S724P, 1586V, E616V, R618G, R618L, D619G, D619R, M621T, S623P, Y628S, K640L, D653A, D653R, D653T, and N666R, wherein the positions are numbered with reference to SEQ ID NO: 984.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 1160, comprising at least one substitution or one substitution set selected from: 132/366/433/463/467/793, 132/366/467/661, 132/467/468/506/793, 132/468/793, 132/1025, 183/1025/1045/1048, 290/366/433/463/467, 290/433/467/793/1025, 290/433/793, 290/793, 290/1025, 366/433, 433, 433/467/1025, 433/506/1025, 433/790, 463/793, 473, 793, and 1025, wherein the positions are numbered with reference to SEQ ID NO: 1160. In some embodiments, the engineered polypeptide comprises at least one substitution or one substitution set selected from 132G/366I/433K/463A/467K/793T, 132G/366I/467G/661D, 132G/467K/468F/506V/793T, 132G/468F/793T, 132G/1025T, 183G/1025A/1045N/1048R, 290T/366I/433K/463A/467K, 290T/433K/467K/793T/1025T, 290T/433K/793T, 290T/793T, 290T/1025T, 366I/433K, 433K, 433K/467K/1025T, 433K/506V/1025T, 433K/790H, 463A/793T, 473L, 793T, and 1025T, wherein the positions are numbered with reference to SEQ ID NO: 1160. In some further embodiments, the engineered polypeptide comprises at least one substitution or one substitution set selected from E132G/T366I/V433K/S463A/S467K/Q793T, E132G/T366I/S467G/G661D, E132G/S467K/A468F/M506V/Q793T, E132G/A468F/Q793T, E132G/S1025T, D183G/S1025A/D1045N/L1048R, K290T/T366I/V433K/S463A/S467K, K290T/V433K/S467K/Q793T/S1025T, K290T/V433K/Q793T, K290T/Q793T, K290T/S1025T, T366I/V433K, V433K, V433K/S467K/S1025T, V433K/M506V/S1025T, V433K/Y790H, S463A/Q793T, K473L, Q793T, and S1025T, wherein the positions are numbered with reference to SEQ ID NO: 1160.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 1266, comprising at least one substitution or one substitution set selected from: 114/230/446/853, 114/292/293/296/463/853, 132/183/366/467/661/1025/1045/1048, 132/290/366/433/467/661/793/1025, 132/290/366/467/661/793, 132/290/366/467/661/1025, 132/366/433/467/506/661/1025, 132/366/433/467/661, 132/366/433/467/661/790, 132/366/463/467/661/793, 132/366/467/473/661, 132/366/467/661/793, 366/467/468/506/661/793, 433/463/467/661/793, 463, 463/853, 689, 720, 724, 730, 769, 780, 792, 810, 817, 824, 853, 923, 926, 939, 952, 962, 968, 974, 979, 981, 995, 1006, 1015, 1017, 1022, 1027, 1031, and 1040, wherein the positions are numbered with reference to SEQ ID NO: 1266. In some embodiments, the engineered polypeptide comprises at least one substitution or one substitution set selected from 114R/230V/446S/853L, 114R/292S/293T/296Q/463V/853L, 132E/183G/366T/467S/661G/1025A/1045N/1048R, 132E/290T/366T/433K/467K/661G/793T/1025T, 132E/290T/366T/467S/661G/793T, 132E/290T/366T/467S/661G/1025T, 132E/366T/433K/467S/506V/661G/1025T, 132E/366T/433K/467S/661G, 132E/366T/433K/467S/661G/790H, 132E/366T/463A/467S/661G/793T, 132E/366T/467S/473L/661G, 132E/366T/467S/661G/793T, 366T/467K/468F/506V/661G/793T, 433K/463A/467K/661G/793T, 463V, 463V/853L, 689G, 720Q, 724W, 730W, 769T, 780I, 792I, 792V, 810S, 817G, 824G, 853L, 923R, 923V, 926R, 939D, 952G, 962R, 968L, 974V, 979Q, 981N, 995G, 1006S, 1015L, 1017A, 1017G, 1017L, 1022S, 1022W, 1027R, 1031Q, 1040D, and 1040R, wherein the positions are numbered with reference to SEQ ID NO: 1266. In some further embodiments, the engineered polypeptide comprises at least one substitution or one substitution set selected from G114R/Q230V/V446S/P853L, G114R/A292S/E293T/A296Q/S463V/P853L, G132E/D183G/I366T/G467S/D661G/S1025A/D1045N/L1048R, G132E/K290T/I366T/V433K/G467K/D661G/Q793T/S1025T, G132E/K290T/I366T/G467S/D661G/Q793T, G132E/K290T/I366T/G467S/D661G/S1025T, G132E/I366T/V433K/G467S/M506V/D661G/S1025T, G132E/I366T/V433K/G467S/D661G, G132E/I366T/V433K/G467S/D661G/Y790H, G132E/I366T/S463A/G467S/D661G/Q793T, G132E/I366T/G467S/K473L/D661G, G132E/I366T/G467S/D661G/Q793T, I366T/G467K/A468F/M506V/D661G/Q793T, V433K/S463A/G467K/D661G/Q793T, S463V, S463V/P853L, L689G, G720Q, S724W, E730W, A769T, E780I, E792I, E792V, A810S, E817G, S824G, P853L, E923R, E923V, Q926R, S939D, N952G, H962R, M968L, T974V, V979Q, E981N, A995G, M1006S, M1015L, S1017A, S1017G, S1017L, H1022S, H1022W, A1027R, L1031Q, G1040D, and G1040R, wherein the positions are numbered with reference to SEQ ID NO: 1266.

The present invention also provides an engineered polypeptide comprising an amino acid sequence with at least 80%, 85%, 86%, 87%, 88%, 89%, 90%, 91%, 92%, 93%, 94%, 95%, 96%, 97%, 98%, 99% sequence identity to a reference sequence of SEQ ID NO: 1266, comprising at least one substitution or one substitution set selected from: 458, 518/653, 519/628, 616/619, and 653, wherein the positions are numbered with reference to SEQ ID NO: 1266. In some embodiments, the engineered polypeptide comprises at least one substitution or one substitution set selected from 458A, 518G/653A, 518G/653R, 519L/628S, 616V/619G, and 653R, wherein the positions are numbered with reference to SEQ ID NO: 1266. In some further embodiments, the engineered polypeptide comprises at least one substitution or one substitution set selected from G458A, D518G/D653A, D518G/D653R, S519L/Y628S, E616V/D619G, and D653R, wherein the positions are numbered with reference to SEQ ID NO: 1266.

In an embodiment, engineered cytochrome P450-BM3 polypeptide variants comprising a polypeptide sequence that is at least 85%, 86%, 87%, 88%, 89%, 90%, 91%, 92%, 93%, 94%, 95%, 96%, 97%, 98%, 99% or more identical to the sequence of at least one engineered cytochrome P450-BM3 variant set forth in Tables 2.1, 3.1, 4.1, 5.1, 6.1, 7.1, 8.1, 9.1, 10.1, 10.2, 11.1, 11.2, 12.1, 13.1, 14.1, 14.2, 15.1, 16.1, 17.1, 18.1, 19.1 and 19.2 are provided.

In various embodiments, the engineered cytochrome P450-BM3 variant comprises at least one improved property as compared to a wild-type Bacillus megaterium cytochrome P450-BM3 or engineered P450-BM3 variant. In some aspects, the improved property comprises improved activity on a substrate. In certain aspects, the substrate comprises 1-tert-Butoxycarbonylaminocyclopentanoic acid (Compound (1)). In some aspects, the improved property comprises improved thermostability or increased activity on a substrate after preincubation at 42.5° C. In some aspects, the improved property comprises improved stereoselectivity toward one or more diastereomer products.

The present invention provides engineered cytochrome P450-BM3 variants wherein the variant is purified. The invention also provides compositions comprising at least one engineered P450-BM3 variant.

The present invention further provides isolated recombinant polynucleotide sequences encoding the recombinant cytochrome P450-BM3 polypeptide variants provided herein. In some embodiments, the isolated recombinant polynucleotide sequence comprises SEQ ID NO: 3, 35, 65, 71, 197, 225, 243, 285, 357, 409, 533, 733, 747, 827, 967, 983, 1159, or 1265, or a functional fragment thereof. In various aspects, the polynucleotide sequence is operably linked to a control sequence. In some aspects, the polynucleotide sequence is codon-optimized. In some embodiments, the polynucleotide sequence comprises a polynucleotide sequence set forth in the odd numbered sequences of SEQ ID NOS:3-1367.

The present invention also provides expression vectors comprising at least one polynucleotide sequence provided herein. In some additional embodiments, the vector comprises at least one polynucleotide sequence that is operably linked with at least one regulatory sequence suitable for expression of the polynucleotide sequence in a suitable host cell. In some embodiments, the host cell is a prokaryotic or eukaryotic cell. In some additional embodiments, the host cell is a prokaryotic cell. In some further embodiments, the host cell is E. coli. The present invention also provides host cells comprising the vectors provided herein.

The present invention also provides methods for producing at least one recombinant cytochrome P450-BM3 variant comprising culturing the host cell provided herein under conditions such that at least one of the recombinant cytochrome P450-BM3 variants provided herein is produced by the host cell. In some additional embodiments, the methods further comprise the step of recovering at least one recombinant cytochrome P450 variant. In some aspect, the methods further comprise the step of purifying the at least one engineered cytochrome P450-BM3 variant.

### DESCRIPTION OF THE INVENTION

The present invention provides improved P450-BM3 variants with improved activity. In some embodiments, the P450-BM3 variants exhibit improved activity on 1-tert-Butoxycarbonylaminocyclopentanoic acid. P450-BM3 enzymes exhibit the highest rate of catalysis amongst P450 monooxygenases due to the efficient electron transfer between the fused reductase and heme domains (See e.g., Noble et al., Biochem. J., 339:371-379 [1999]; and Munro et al., Eur. J. Biochem., 239:403-409 [2009]). Thus, P450-BM3 is a highly desirable enzyme for the manipulation of biotechnological processes (See e.g., Sawayama et al., Chem., 15:11723-11729 [2009]; Otey et al., Biotechnol. Bioeng., 93:494-499 [2006]; Damsten et al., Biol. Interact., 171:96-107 [2008]; and Di Nardo and Gilardi, Int. J. Mol. Sci., 13:15901-15924). However, there still remains a need in the art for P450 enzymes that exhibit activity on various substrates, including 1-tert-Butoxycarbonylaminocyclopentanoic acid. The present invention provides P450-BM3 variants that have improved enzymatic activity on 1-tert-Butoxycarbonylaminocyclopentanoic acid, as compared to a parental P450-BM3 sequence (i.e., SEQ ID NO: 4, 36, 66, 72, 198, 226, 244, 286, 358, 410, 534, 734, 748, 828, 968, 984, 1160, or 1266).

In some embodiments, the present invention provides P450-BM3 variants that provide improved total percent conversion/activity for the oxidation of substrates. In some embodiments, the present invention provides P450-BM3 variants that provide improved thermostability in the oxidation of substrates. In some embodiments, the present invention provides P450-BM3 variants that provide improved stereoselectivity towards various diastereomers in the oxidation of substrates. In particular, during the development of the present invention, beneficial diversity was identified and recombined based on HTP screening results.

### Abbreviations and Definitions:

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses. All patents, patent applications, articles and publications mentioned herein, both supra and infra, are hereby expressly incorporated herein by reference.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the application as a whole. All patents, patent applications, articles and publications mentioned herein, both supra and infra, are hereby expressly incorporated herein by reference.

Also, as used herein, the singular “a”, “an,” and “the” include the plural references, unless the context clearly indicates otherwise.

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

The term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole. Nonetheless, in order to facilitate understanding of the invention, a number of terms are defined below.

Unless otherwise indicated, nucleic acids are written left to right in 5′ to 3′ orientation; amino acid sequences are written left to right in amino to carboxy orientation, respectively.

As used herein, the term “comprising” and its cognates are used in their inclusive sense (i.e., equivalent to the term “including” and its corresponding cognates).

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

As used herein “cytochrome P450-BM3” and “P450-BM3” refer to the cytochrome P450 enzyme obtained from Bacillus megaterium (now also known as Priestia megaterium) that catalyzes the NADPH-dependent hydroxylation of long-chain fatty acids, alcohols, and amides, as well as the epoxidation of unsaturated fatty acids

“Protein,” “polypeptide,” and “peptide” are used interchangeably herein to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

The term “engineered,” “recombinant,” “non-naturally occurring,” and “variant,” when used with reference to a cell, a polynucleotide or a polypeptide refers to a material or a material corresponding to the natural or native form of the material that has been modified in a manner that would not otherwise exist in nature or is identical thereto but produced or derived from synthetic materials and/or by manipulation using recombinant techniques.

As used herein, “wild-type” and “naturally-occurring” refer to the form found in nature. For example a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein.

The term “percent (%) sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 2:482 [1981]), by the homology alignment algorithm of Needleman and Wunsch (Needleman and Wunsch, J. Mol. Biol., 48:443 [1970), by the search for similarity method of Pearson and Lipman (Pearson and Lipman, Proc. Natl. Acad. Sci. USA 85:2444 [1988]), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol., 215: 403-410 [1990]; and Altschul et al., 1977, Nucl. Acids Res., 3389-3402 [1977], respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (See, Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See, Henikoff and Henikoff, Proc. Natl. Acad. Sci. USA 89:10915 [1989]). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. “Comparison window” refers to a conceptual segment of at least about 20 contiguous nucleotide positions or amino acid residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered P450-BM3, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Amino acid difference” or “residue difference” refers to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X93 as compared to SEQ ID NO:2” refers to a difference of the amino acid residue at the polypeptide position corresponding to position 93 of SEQ ID NO:2. Thus, if the reference polypeptide of SEQ ID NO:2 has a serine at position 93, then a “residue difference at position X93 as compared to SEQ ID NO:2” an amino acid substitution of any residue other than serine at the position of the polypeptide corresponding to position 93 of SEQ ID NO:2. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances (e.g., in Tables 2.1, 3.1, 4.1, 5.1, 6.1, 7.1, 8.1, 9.1, 10.1, 10.2, 11.1, 11.2, 12.1, 13.1, 14.1, 14.2, 15.1, 16.1, 17.1, 17.2, 18.1, 19.1, and 19.2), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X307H/X307P or X307H/P). The present application includes engineered polypeptide sequences comprising one or more amino acid differences that include either/or both conservative and non-conservative amino acid substitutions.

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acid having an aromatic side chain is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered P450-BM3 enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

A “functional fragment” or a “biologically active fragment” used interchangeably herein refers to a polypeptide that has an amino-terminal and/or carboxy-terminal deletion(s) and/or internal deletions, but where the remaining amino acid sequence is identical to the corresponding positions in the sequence to which it is being compared (e.g., a full-length engineered P450-BM3 of the present invention) and that retains substantially all of the activity of the full-length polypeptide.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The recombinant P450-BM3 polypeptides may be present within a cell, present in the cellular medium, or prepared in various forms, such as lysates or isolated preparations. As such, in some embodiments, the recombinant P450-BM3 polypeptides can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. However, in some embodiments, the composition comprising P450-BM3 comprises P450-BM3 that this less than 50% pure (e.g., about 10%, about 20%, about 30%, about 40%, or about 50%) Generally, a substantially pure P450-BM3 composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant P450-BM3 polypeptides are substantially pure polypeptide compositions.

“Improved enzyme property” refers to an engineered P450-BM3 polypeptide that exhibits an improvement in any enzyme property as compared to a reference P450-BM3 polypeptide and/or a wild-type P450-BM3 polypeptide or another engineered P450-BM3 polypeptide. Improved properties include but are not limited to such properties as increased protein expression, increased thermoactivity, increased thermostability, increased pH activity, increased stability, increased enzymatic activity, increased substrate specificity or affinity, increased specific activity, increased resistance to substrate or end-product inhibition, increased chemical stability, improved stereoselectivity, improved chemoselectivity, improved solvent stability, increased tolerance to acidic pH, increased tolerance to proteolytic activity (i.e., reduced sensitivity to proteolysis), reduced aggregation, increased solubility, and altered temperature profile.

“Increased enzymatic activity” or “enhanced catalytic activity” refers to an improved property of the engineered P450-BM3 polypeptides, which can be represented by an increase in specific activity (e.g., product produced/time/weight protein) or an increase in percent conversion of the substrate to the product (e.g., percent conversion of starting amount of substrate to product in a specified time period using a specified amount of P450-BM3) as compared to the reference P450-BM3 enzyme. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.1 fold the enzymatic activity of the corresponding wild-type enzyme, to as much as 2-fold, 5-fold, 10-fold, 20-fold, 25-fold, 50-fold, 75-fold, 100-fold, 150-fold, 200-fold or more enzymatic activity than the naturally occurring P450-BM3 or another engineered P450-BM3 from which the P450-BM3 polypeptides were derived.

“Conversion” refers to the enzymatic conversion (or biotransformation) of a substrate(s) to the corresponding product(s). “Percent conversion” refers to the percent of the substrate that is converted to the product within a period of time under specified conditions. Thus, the “enzymatic activity” or “activity” of a P450-BM3 polypeptide can be expressed as “percent conversion” of the substrate to the product in a specific period of time.

Enzymes with “generalist properties” (or “generalist enzymes”) refer to enzymes that exhibit improved activity for a wide range of substrates, as compared to a parental sequence. Generalist enzymes do not necessarily demonstrate improved activity for every possible substrate. In particular, the present invention provides P450-BM3 variants with generalist properties, in that they demonstrate similar or improved activity relative to the parental gene for a wide range of sterically and electronically diverse substrates. In addition, the generalist enzymes provided herein were engineered to be improved across a wide range of diverse API-like molecules to increase the production of metabolites/products.

“Hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature T, as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the P450-BM3 enzymes may be codon optimized for optimal production from the host organism selected for expression.

“Control sequence” refers herein to include all components, which are necessary or advantageous for the expression of a polynucleotide and/or polypeptide of the present application. Each control sequence may be native or foreign to the nucleic acid sequence encoding the polypeptide. Such control sequences include, but are not limited to, a leader, polyadenylation sequence, propeptide sequence, promoter sequence, signal peptide sequence, initiation sequence and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” refers to those conditions in the enzymatic conversion reaction solution (e.g., ranges of enzyme loading, substrate loading, temperature, pH, buffers, co-solvents, etc.) under which a P450-BM3 polypeptide of the present application is capable of converting a substrate to the desired product compound, Exemplary “suitable reaction conditions” are provided in the present application and illustrated by the Examples. “Loading”, such as in “compound loading” or “enzyme loading” refers to the concentration or amount of a component in a reaction mixture at the start of the reaction. “Substrate” in the context of an enzymatic conversion reaction process refers to the compound or molecule acted on by the P450-BM3 polypeptide. “Product” in the context of an enzymatic conversion process refers to the compound or molecule resulting from the action of the P450-BM3 polypeptide on a substrate.

As used herein the term “culturing” refers to the growing of a population of microbial cells under any suitable conditions (e.g., using a liquid, gel or solid medium).

Recombinant polypeptides can be produced using any suitable methods known in the art. Genes encoding the wild-type polypeptide of interest can be cloned in vectors, such as plasmids, and expressed in desired hosts, such as E. coli, etc. Variants of recombinant polypeptides can be generated by various methods known in the art. Indeed, there are a wide variety of different mutagenesis techniques well known to those skilled in the art. In addition, mutagenesis kits are also available from many commercial molecular biology suppliers. Methods are available to make specific substitutions at defined amino acids (site-directed), specific or random mutations in a localized region of the gene (regio-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased activity, or low or reduced activity, increased thermal activity, increased thermal stability, and/or acidic pH stability, etc.). In some embodiments, “recombinant P450-BM3 polypeptides” (also referred to herein as “engineered P450-BM3 polypeptides,” “variant P450-BM3 enzymes,” and “P450-BM3 variants”) find use.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature.

As used herein, the terms “host cell” and “host strain” refer to suitable hosts for expression vectors comprising DNA provided herein (e.g., the polynucleotides encoding the P450-BM3 variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, analogues mean polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

### Engineered P450-BM3 Polypeptides:

The present invention provides improved P450-BM3 variants with improved activity on 1-tert-Butoxycarbonylaminocyclopentanoic acid.

In some embodiments, the present disclosure provides P450-BM3 variants with improved monooxygenase activity towards 1-tert-Butoxycarbonylaminocyclopentanoic acid (Compound (1)) and increased conversion to the product of 1-(tert-Butoxycarbonylamino)-3-hydroxycyclopentanoic acid (Compound (2)) and/or 1-(tert-Butoxycarbonylamino)-3-oxocyclopentanoic acid (Compound (3)), as compared to the starting polypeptide, as depicted in Scheme 1.

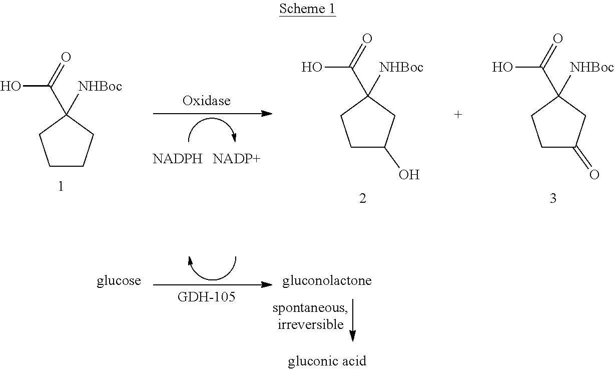 [SMILES: CC(C)(C)OC(=O)NC1(C(=O)O)CCCC1 ; CC(C)(C)OC(=O)NC1(C(=O)O)CCC(O)C1 ; CC(C)(C)OC(=O)NC1(C(=O)O)CCC(=O)C1]

In some embodiments, the monooxygenase (also referred to as oxidase) reaction uses NADPH as a cofactor, which is recycled from NADP+ by a glucose dehydrogenase (GDH-105, Codexis, Inc.) as depicted above, in Scheme 1. However, the use of the GDH recycling system can cause a downward shift in reaction pH. In some embodiments, a phosphite dehydrogenase (PDH-102, Codexis, Inc.) is used for cofactor recycling, as depicted, below, in Scheme 2.

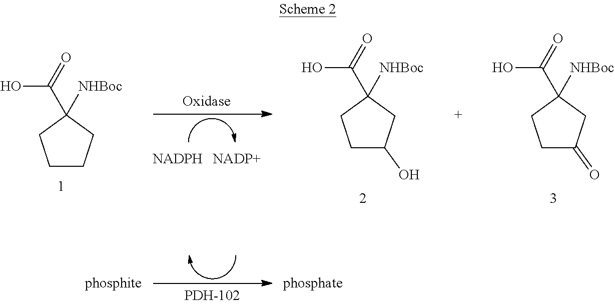 [SMILES: CC(C)(C)OC(=O)NC1(C(=O)O)CCCC1 ; CC(C)(C)OC(=O)NC1(C(=O)O)CCC(O)C1 ; CC(C)(C)OC(=O)NC1(C(=O)O)CCC(=O)C1]

The P450-BM3 variants of the present disclosure produce the four diastereoisomers of Compound (2) depicted below in Scheme 3. Additionally, hydroxylation has also been observed at position 4, yielding additional diastereomers represented as Compound (4), below in Scheme 3.

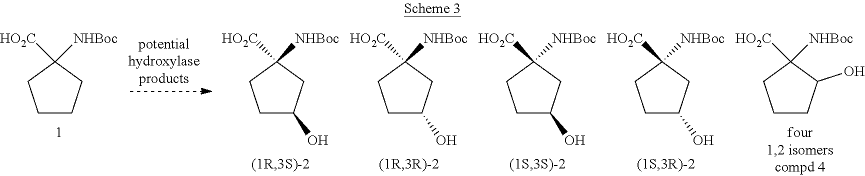

In some embodiments, the engineered P450-BM3 polypeptides of the present disclosure are regioselective for hydroxylation at the 2 position as compared to a reference P450-BM3 polypeptide. In some embodiments, the engineered P450-BM3 polypeptides of the present disclosure are stereoselective for the (1R,3S)-2 and/or (1R,3R)-2 diastereoisomers as compared to a reference P450-BM3 polypeptide.

In some embodiments, the engineered P450-BM3 polypeptides of the present disclosure may produce ketone products from the substrate of Compound (1), as depicted below, in Scheme 4.

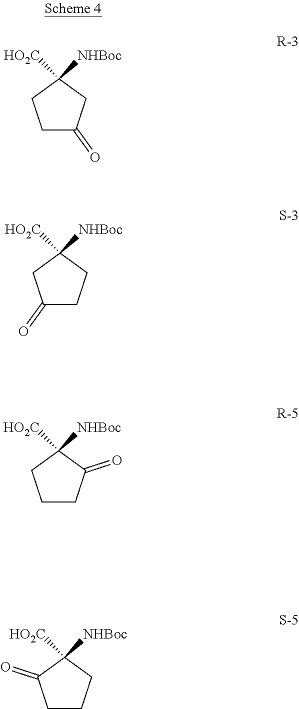

In some embodiments, the engineered P450-BM3 polypeptides of the present disclosure are regioselective for oxidation to a ketone at the 3 position as compared to a reference P450-BM3 polypeptide.

The present invention provides exemplary engineered P450-BM3 polypeptides having P450-BM3 activity (i.e., P450-BM3 variants). The Examples provide Tables showing sequence structural information correlating specific amino acid sequence features with the functional activity of the engineered P450-BM3 polypeptides. This structure-function correlation information is provided in the form of specific amino acid residues differences relative to a reference engineered polypeptide, as indicated in the Examples. The Examples further provide experimentally determined activity data for the exemplary engineered P450-BM3 polypeptides.

In some embodiments, the engineered P450-BM3 polypeptides of the invention having P450-BM3 activity comprise: a) an amino acid sequence having at least 80% sequence identity to reference sequence SEQ ID NO: 4, 36, 66, 72, 198, 226, 244, 286, 358, 410, 534, 734, 748, 828, 968, 984, 1160, or 1266; b) an amino acid residue difference as compared to SEQ ID NO: 4, 36, 66, 72, 198, 226, 244, 286, 358, 410, 534, 734, 748, 828, 968, 984, 1160, or 1266 at one or more amino acid positions; and c) which exhibits an improved property selected from i) enhanced catalytic activity, ii) increased thermostability, iii) increased tolerance to acidic pH, iv) reduced aggregation, v) increased activity on 1-tert-Butoxycarbonylaminocyclopentanoic acid substrates, vi) increased solubility, vii) increased regioselectivity, or viii) increased selectivity toward the desired chiral product or a combination of any of i), ii), iii), iv), v), vi), vii), or viii), as compared to the reference sequence.

In some embodiments the engineered P450-BM3 which exhibits an improved property has at least about 85%, at least about 88%, at least about 90%, at least about 91%, at least about 92%, at least about 93%, at least about 94%, at least about 95%, at least about 96%, at least about 97%, at least about 98%, at least about 99%, or at about 100% amino acid sequence identity with SEQ ID NO: 4, 36, 66, 72, 198, 226, 244, 286, 358, 410, 534, 734, 748, 828, 968, 984, 1160, or 1266, and an amino acid residue difference as compared to SEQ ID NO: 4, 36, 66, 72, 198, 226, 244, 286, 358, 410, 534, 734, 748, 828, 968, 984, 1160, or 1266, at one or more amino acid positions (such as at 1, 2, 3, 4, 5, 6, 7, 8, 9, 10, 11, 12, 14, 15, 20 or more amino acid positions compared to SEQ ID NO: 4, 36, 66, 72, 198, 226, 244, 286, 358, 410, 534, 734, 748, 828, 968, 984, 1160, or 1266, or a sequence having at least 85%, at least 88%, at least 90%, at least 91%, at least 92%, at least 93%, at least 94%, at least 95%, at least 96%, at least 97%, at least 98%, at least 99% or greater amino acid sequence identity with SEQ ID NO: 4, 36, 66, 72, 198, 226, 244, 286, 358, 410, 534, 734, 748, 828, 968, 984, 1160, or 1266). In some embodiments, the residue difference as compared to SEQ ID NO: 4, 36, 66, 72, 198, 226, 244, 286, 358, 410, 534, 734, 748, 828, 968, 984, 1160, or 1266, at one or more positions will include at least 1, 2, 3, 4, 5, 6, 7, 8, 9, 10 or more conservative amino acid substitutions. In some embodiments, the engineered P450-BM3 polypeptide is a polypeptide listed in any of Tables 2.1, 3.1, 4.1, 5.1, 6.1, 7.1, 8.1, 9.1, 10.1, 10.2, 11.1, 11.2, 12.1, 13.1, 14.1, 14.2, 15.1, 16.1, 17.1, 17.2, 18.1, 19.1, and 19.2.

In some embodiments the engineered P450-BM3 which exhibits an improved property has at least 85%, at least 88%, at least 90%, at least 91%, at least 92%, at least 93%, at least 94%, at least 95%, at least 96%, at least 97%, at least 98%, or at least 99% amino acid sequence identity with SEQ ID NO: 4, 36, 66, 72, 198, 226, 244, 286, 358, 410, 534, 734, 748, 828, 968, 984, 1160, or 1266.

In some embodiments, the engineered P450-BM3 polypeptides of the present disclosure comprise an amino acid sequence having at least 85%, 90%, 95%, or 99% sequence identity to a reference sequence of SEQ ID NO: 4, 36, 66, 72, 198, 226, 244, 286, 358, 410, 534, 734, 748, 828, 968, 984, 1160, or 1266 and an amino acid residue difference as compared to SEQ ID NO: 4, 36, 66, 72, 198, 226, 244, 286, 358, 410, 534, 734, 748, 828, 968, 984, 1160, or 1266 at one or more amino acid positions, wherein said engineered P450-BM3 polypeptide converts 1-tert-Butoxycarbonylaminocyclopentanoic acid (Compound (1)) to the product of 1-(tert-Butoxycarbonylamino)-3-hydroxycyclopentanoic acid (Compound (2)) and/or 1-(tert-Butoxycarbonylamino)-3-oxocyclopentanoic acid (Compound (3)), as compared to the starting polypeptide with at least 1.5 fold, 2.0 fold, 5.0 fold, 10.0 fold, 50.0 fold, 100.0 fold, 200.0 fold, 300.0 fold, 400.0 fold or 500.0 fold the activity of a reference engineered P450-BM3 polypeptide of SEQ ID NO: 4, 36, 66, 72, 198, 226, 244, 286, 358, 410, 534, 734, 748, 828, 968, 984, 1160, or 1266.

In some embodiments, the engineered P450-BM3 polypeptides of the present disclosure comprise an amino acid sequence having at least 85%, 90%, 95%, or 99% sequence identity to a reference sequence of SEQ ID NO: 4, 36, 66, 72, 198, 226, 244, 286, 358, 410, 534, 734, 748, 828, 968, 984, 1160, or 1266 and an amino acid residue difference as compared to SEQ ID NO: 4, 36, 66, 72, 198, 226, 244, 286, 358, 410, 534, 734, 748, 828, 968, 984, 1160, or 1266 at one or more amino acid positions, wherein said engineered P450-BM3 polypeptide converts 1-tert-Butoxycarbonylaminocyclopentanoic acid (Compound (1)) to the product of 1-(tert-Butoxycarbonylamino)-3-hydroxycyclopentanoic acid (Compound (2)) and/or 1-(tert-Butoxycarbonylamino)-3-oxocyclopentanoic acid (Compound (3)) with increased thermostability or increased activity after preincubation at an increased temperature (such as 42.5° C.) as compared to a reference engineered P450-BM3 polypeptide of SEQ ID NO: 4, 36, 66, 72, 198, 226, 244, 286, 358, 410, 534, 734, 748, 828, 968, 984, 1160, or 1266.

In some embodiments, the engineered P450-BM3 polypeptides of the present disclosure comprise an amino acid sequence having at least 85%, 90%, 95%, or 99% sequence identity to a reference sequence of SEQ ID NO: 4, 36, 66, 72, 198, 226, 244, 286, 358, 410, 534, 734, 748, 828, 968, 984, 1160, or 1266 and an amino acid residue difference as compared to SEQ ID NO: 4, 36, 66, 72, 198, 226, 244, 286, 358, 410, 534, 734, 748, 828, 968, 984, 1160, or 1266 at one or more amino acid positions, wherein said engineered P450-BM3 polypeptide converts 1-tert-Butoxycarbonylaminocyclopentanoic acid (Compound (1)) to the product of 1-(tert-Butoxycarbonylamino)-3-hydroxycyclopentanoic acid (Compound (2)) and/or 1-(tert-Butoxycarbonylamino)-3-oxocyclopentanoic acid (Compound (3)) with at least 5%, 20%, 30%, 50%, 75%, 80%, or 90% enantiomeric excess for Compound (2) or Compound (3), as compared to a reference engineered P450-BM3 polypeptide of SEQ ID NO: 4, 36, 66, 72, 198, 226, 244, 286, 358, 410, 534, 734, 748, 828, 968, 984, 1160, or 1266.

In some embodiments, the engineered P450-BM3 polypeptide comprises a functional fragment of an engineered P450-BM3 polypeptide encompassed by the invention. Functional fragments have at least 95%, 96%, 97%, 98%, or 99% of the activity of the engineered P450-BM3 polypeptide from which is derived (i.e., the parent engineered P450-BM3). A functional fragment comprises at least 90%, 91%, 92%, 93%, 94%, 95%, 96%, 97%, 98% and even 99% of the parent sequence of the engineered P450-BM3. In some embodiments the functional fragment is truncated by less than 5, less than 10, less than 15, less than 10, less than 25, less than 30, less than 35, less than 40, less than 45, and less than 50 amino acids.

### Methods of Using the Engineered P450-BM3 Polypeptides Enzymes:

In some embodiments, the engineered P450-BM3 polypeptides of the invention having P450-BM3 activity comprise: a) an amino acid sequence having at least 85% sequence identity to reference sequence SEQ ID NO: 4, 36, 66, 72, 198, 226, 244, 286, 358, 410, 534, 734, 748, 828, 968, 984, 1160, or 1266, or a fragment thereof; b) an amino acid residue difference as compared to SEQ ID NO: 4, 36, 66, 72, 198, 226, 244, 286, 358, 410, 534, 734, 748, 828, 968, 984, 1160, or 1266, at one or more amino acid positions; and c) which exhibits improved activity, as compared to SEQ ID NO: 4, 36, 66, 72, 198, 226, 244, 286, 358, 410, 534, 734, 748, 828, 968, 984, 1160, or 1266.

In some embodiments, the engineered P450-BM3 that exhibits improved activity has at least 85%, at least 88%, at least 90%, at least 91%, at least 92%, at least 93%, at least 94%, at least 95%, at least 96%, at least 97%, at least 98%, at least 99% or greater amino acid sequence identity with SEQ ID NO: 4, 36, 66, 72, 198, 226, 244, 286, 358, 410, 534, 734, 748, 828, 968, 984, 1160, or 1266, and an amino acid residue difference as compared to SEQ ID NO: 4, 36, 66, 72, 198, 226, 244, 286, 358, 410, 534, 734, 748, 828, 968, 984, 1160, or 1266, at one or more amino acid positions (such as at 1, 2, 3, 4, 5, 6, 7, 8, 9, 10, 11, 12, 14, 15, 20 or more amino acid positions compared to SEQ ID NO: 4, 36, 66, 72, 198, 226, 244, 286, 358, 410, 534, 734, 748, 828, 968, 984, 1160, or 1266, or a sequence having at least 85%, at least 88%, at least 90%, at least 91%, at least 92%, at least 93%, at least 94%, at least 95%, at least 96%, at least 97%, at least 98%, at least 99% or greater amino acid sequence identity with SEQ ID NO: 4, 36, 66, 72, 198, 226, 244, 286, 358, 410, 534, 734, 748, 828, 968, 984, 1160, or 1266.

In some embodiments, when all other assay conditions are essentially the same, the engineered P450-BM3 polypeptide has improved activity as compared to a reference P450-BM3 polypeptide. In some embodiments this activity can be measured under conditions that monitor enzymatic activity using any suitable assay system to assess the maximum activity of the enzyme (e.g., the k_{cat}). In other embodiments this activity can be measured under substrate concentrations resulting in one-half, one-fifth, one-tenth or less of maximal activity. Under either method of analysis, the engineered polypeptide has improved activity levels about 1.0 fold, 1.5-fold, 2-fold, 5-fold, 10-fold, 20-fold, 25-fold, 50-fold, 75-fold, 100-fold, or more of the enzymatic activity of the reference P450-BM3 In some embodiments, the engineered P450-BM3 polypeptide having improved activity as compared to a reference P450-BM3 when measured by any standard assay, including, but not limited to the assays described in the Examples.

In some embodiments, the engineered P450-BM3 polypeptides described herein find use in processes for converting 1-tert-Butoxycarbonylaminocyclopentanoic acid (Compound (1)) to the product of 1-(tert-Butoxycarbonylamino)-3-hydroxycyclopentanoic acid (Compound (2)) and/or 1-(tert-Butoxycarbonylamino)-3-oxocyclopentanoic acid (Compound (3)). Generally, the process for performing the monooxygenation reaction comprises contacting or incubating the substrate compound in presence of a co-substrate, such as NADP+, with an engineered P450-BM3 polypeptide of the invention under reaction conditions suitable for formation of the hydroxylated product, as shown in Scheme 1 and Scheme 2, above.

In the embodiments provided herein and illustrated in the Examples, various ranges of suitable reaction conditions that can be used in the processes, include but are not limited to, substrate loading, reductant, recycling system, pH, temperature, buffer, solvent system, polypeptide loading, and reaction time. Further suitable reaction conditions for carrying out the process for biocatalytic conversion of substrate compounds to product compounds using an engineered P450-BM3 polypeptide described herein can be readily optimized in view of the guidance provided herein by routine experimentation that includes, but is not limited to, contacting the engineered P450-BM3 polypeptide and substrate compound under experimental reaction conditions of concentration, pH, temperature, and solvent conditions, and detecting the product compound.

Suitable reaction conditions using the engineered P450-BM3 polypeptides typically comprise a NADP+co-substrate, which is used stoichiometrically in the monooxygenation reaction. Generally, the co-substrate for engineered P450-BM3 polypeptide is NADP+. Other reductants that are capable of serving as co-substrates for engineered P450-BM3 polypeptides can be used. In some embodiments, the suitable reaction conditions can comprise a co-substrate concentration, particularly NADP+ of about 0.0005 M to about 2 M, 0.01 M to about 2 M, 0.1 M to about 2 M, 0.2 M to about 2 M, about 0.5 M to about 2 M, or about 1 M to about 2 M. In some embodiments, the reaction conditions comprise a co-substrate concentration of about 0.0001 M, 0.001 M, 0.01 M, 0.1 M, 0.2 M, 0.3 M, 0.4 M, 0.5 M, 0.6 M, 0.7 M, 0.8 M, 1 M, 1.5 M, or 2 M, depending on desired conversion. In some embodiments, additional co-substrate can be added during the reaction. In some embodiments, an NADP+ recycling system can be used in the reaction.

Substrate compound in the reaction mixtures can be varied, taking into consideration, for example, the desired amount of product compound, the effect of substrate concentration on enzyme activity, stability of enzyme under reaction conditions, and the percent conversion of substrate to product. In some embodiments, the suitable reaction conditions comprise a substrate compound loading of at least about 0.5 to about 200 g/L, 1 to about 200 g/L, 5 to about 150 g/L, about 10 to about 100 g/L, 20 to about 100 g/L or about 50 to about 100 g/L. In some embodiments, the suitable reaction conditions comprise a substrate compound loading of at least about 0.5 g/L, at least about 1 g/L, at least about 5 g/L, at least about 10 g/L, at least about 15 g/L, at least about 20 g/L, at least about 30 g/L, at least about 50 g/L, at least about 75 g/L, at least about 100 g/L, at least about 150 g/L or at least about 200 g/L, or even greater.

In carrying out the engineered P450-BM3 mediated processes described herein, the engineered polypeptide may be added to the reaction mixture in the form of a purified enzyme, partially purified enzyme, whole cells transformed with gene(s) encoding the enzyme, as cell extracts and/or lysates of such cells, and/or as an enzyme immobilized on a solid support. Whole cells transformed with gene(s) encoding the engineered P450-BM3 enzyme or cell extracts, lysates thereof, and isolated enzymes may be employed in a variety of different forms, including solid (e.g., lyophilized, spray-dried, and the like) or semisolid (e.g., a crude paste). The cell extracts or cell lysates may be partially purified by precipitation (ammonium sulfate, polyethyleneimine, heat treatment or the like, followed by a desalting procedure prior to lyophilization (e.g., ultrafiltration, dialysis, etc.). Any of the enzyme preparations (including whole cell preparations) may be stabilized by crosslinking using known crosslinking agents, such as, for example, glutaraldehyde or immobilization to a solid phase (e.g., Eupergit C, and the like).

The gene(s) encoding the engineered P450-BM3 polypeptides can be transformed into host cells separately or together into the same host cell. For example, in some embodiments one set of host cells can be transformed with gene(s) encoding one engineered P450-BM3 polypeptide and another set can be transformed with gene(s) encoding another engineered P450-BM3 polypeptide. Both sets of transformed cells can be utilized together in the reaction mixture in the form of whole cells, or in the form of lysates or extracts derived therefrom. In other embodiments, a host cell can be transformed with gene(s) encoding multiple engineered P450-BM3 polypeptides. In some embodiments the engineered polypeptides can be expressed in the form of secreted polypeptides, and the culture medium containing the secreted polypeptides can be used for the P450-BM3 reaction.

In some embodiments, the improved activity and/or selectivity of the engineered P450-BM3 polypeptides disclosed herein provides for processes wherein higher percentage conversion can be achieved with lower concentrations of the engineered polypeptide. In some embodiments of the process, the suitable reaction conditions comprise an engineered polypeptide amount of about 0.03% (w/w), 0.05% (w/w), 0.1% (w/w), 0.15% (w/w), 0.2% (w/w), 0.3% (w/w), 0.4% (w/w), 0.5% (w/w), 1% (w/w), 2% (w/w), 5% (w/w), 10% (w/w), 20% (w/w) or more, depending on the substrate compound loading and desired conversion.

In some embodiments, the engineered polypeptide is present at about 0.01 g/L to about 40 g/L; about 0.05 g/L to about 15 g/L; about 0.1 g/L to about 10 g/L; about 1 g/L to about 8 g/L; about 0.5 g/L to about 10 g/L; about 1 g/L to about 10 g/L; about 0.1 g/L to about 5 g/L; about 0.5 g/L to about 5 g/L; or about 0.1 g/L to about 2 g/L. In some embodiments, the engineered P450-BM3 polypeptide is present at about 0.01 g/L, 0.05 g/L, 0.1 g/L, 0.2 g/L, 0.5 g/L, 1 g/L, 2 g/L, 5 g/L, 10 g/L, 15 g/L, 20 g/L, 40 g/L or more.

During the course of the reaction, the pH of the reaction mixture may change. The pH of the reaction mixture may be maintained at a desired pH or within a desired pH range. This may be done by the addition of an acid or a base, before and/or during the course of the reaction. Alternatively, the pH may be controlled by using a buffer. Accordingly, in some embodiments, the reaction condition comprises a buffer. Suitable buffers to maintain desired pH ranges are known in the art and include, by way of example and not limitation, potassium phosphate, borate, phosphate, 2-(N-morpholino)ethanesulfonic acid (MES), 3-(N-morpholino)propanesulfonic acid (MOPS), acetate, triethanolamine, and 2-amino-2-hydroxymethyl-propane-1,3-diol (Tris), and the like. In some embodiments, the buffer is potassium phosphate. In some embodiments of the process, the suitable reaction conditions comprise a buffer concentration of from about 0.01 to about 0.5 M, 0.05 to about 0.4 M, 0.1 to about 0.3 M, or about 0.1 to about 0.2 M. In some embodiments, the reaction condition comprises a buffer concentration of about 0.01, 0.02, 0.03, 0.04, 0.05, 0.07, 0.1, 0.12, 0.14, 0.16, 0.18, 0.2, 0.3, 0.4 M, or 0.5M.

In some embodiments, the reaction conditions comprise a solvent. Any suitable solvent may be used. In some embodiments, the solvent comprises acetonitrile or DMSO. In some embodiments, the reaction conditions comprise a solvent concentration of 2%, 5%, 20%, 15%, 20%, 25%, or 30%.

In the embodiments of the process, the reaction conditions can comprise a suitable pH. The desired pH or desired pH range can be maintained by use of an acid or base, an appropriate buffer, or a combination of buffering and acid or base addition. The pH of the reaction mixture can be controlled before and/or during the course of the reaction. In some embodiments, the suitable reaction conditions comprise a solution pH from about 4 to about 10, pH from about 5 to about 10, pH from about 5 to about 9, pH from about 6 to about 9, pH from about 6 to about 8. In some embodiments, the reaction conditions comprise a solution pH of about 4, 4.5, 5, 5.5, 6, 6.5, 7, 7.5, 8, 8.5, 9, 9.5, or 10.

In the embodiments of the processes herein, a suitable temperature can be used for the reaction conditions, for example, taking into consideration the increase in reaction rate at higher temperatures, and the activity of the enzyme during the reaction time period. Accordingly, in some embodiments, the suitable reaction conditions comprise a temperature of about 10° C. to about 60° C., about 10° C. to about 55° C., about 15° C. to about 60° C., about 20° C. to about 60° C., about 20° C. to about 55° C., about 25° C. to about 55° C., or about 30° C. to about 50° C. In some embodiments, the suitable reaction conditions comprise a temperature of about 10° C., 15° C., 20° C., 25° C., 30° C., 35° C., 40° C., 45° C., 50° C., 55° C., or 60° C. In some embodiments, the temperature during the enzymatic reaction can be maintained at a specific temperature throughout the course of the reaction. In some embodiments, the temperature during the enzymatic reaction can be adjusted over a temperature profile during the course of the reaction.

In some embodiments, the reaction conditions can comprise a surfactant for stabilizing or enhancing the reaction. Surfactants can comprise non-ionic, cationic, anionic and/or amphiphilic surfactants. Exemplary surfactants, include by way of example and not limitation, nonyl phenoxypolyethoxylethanol (NP40), Triton X-100, polyoxyethylene-stearylamine, cetyltrimethylammonium bromide, sodium oleylamidosulfate, polyoxyethylene-sorbitanmonostearate, hexadecyldimethylamine, etc. Any surfactant that may stabilize or enhance the reaction may be employed. The concentration of the surfactant to be employed in the reaction may be generally from 0.1 to 50 mg/ml, particularly from 1 to 20 mg/ml.

In some embodiments, the reaction conditions can include an antifoam agent, which aids in reducing or preventing formation of foam in the reaction solution, such as when the reaction solutions are mixed or sparged. Anti-foam agents include non-polar oils (e.g., minerals, silicones, etc.), polar oils (e.g., fatty acids, alkyl amines, alkyl amides, alkyl sulfates, etc.), and hydrophobic (e.g., treated silica, polypropylene, etc.), some of which also function as surfactants. Exemplary anti-foam agents include, Y-30® (Dow Corning), poly-glycol copolymers, oxy/ethoxylated alcohols, and polydimethylsiloxanes. In some embodiments, the anti-foam can be present at about 0.001% (v/v) to about 5% (v/v), about 0.01% (v/v) to about 5% (v/v), about 0.1% (v/v) to about 5% (v/v), or about 0.1% (v/v) to about 2% (v/v). In some embodiments, the anti-foam agent can be present at about 0.001% (v/v), about 0.01% (v/v), about 0.1% (v/v), about 0.5% (v/v), about 1% (v/v), about 2% (v/v), about 3% (v/v), about 4% (v/v), or about 5% (v/v) or more as desirable to promote the reaction.

The quantities of reactants used in the monooxygenase reaction will generally vary depending on the quantities of product desired, and concomitantly the amount of indanone substrate employed. Those having ordinary skill in the art will readily understand how to vary these quantities to tailor them to the desired level of productivity and scale of production.

In some embodiments, the order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor, co-substrate, engineered P450-BM3 enzyme, and substrate may be added first to the solvent.

The solid reactants (e.g., enzyme, salts, etc.) may be provided to the reaction in a variety of different forms, including powder (e.g., lyophilized, spray dried, and the like), solution, emulsion, suspension, and the like. The reactants can be readily lyophilized or spray dried using methods and equipment that are known to those having ordinary skill in the art. For example, the protein solution can be frozen at −80° C. in small aliquots, then added to a pre-chilled lyophilization chamber, followed by the application of a vacuum.

For improved mixing efficiency when an aqueous co-solvent system is used, the engineered P450-BM3 polypeptide, and cofactor may be added and mixed into the aqueous phase first. The organic phase may then be added and mixed in, followed by addition of the substrate and co-substrate. Alternatively, the substrate may be premixed in the organic phase, prior to addition to the aqueous phase.

The monooxygenation process is generally allowed to proceed until further conversion of substrate to product does not change significantly with reaction time (e.g., less than 10% of substrate being converted, or less than 5% of substrate being converted). In some embodiments, the reaction is allowed to proceed until there is complete or near complete conversion of substrate to product. Transformation of substrate to product can be monitored using known methods by detecting substrate and/or product, with or without derivatization. Suitable analytical methods include gas chromatography, HPLC, MS, and the like.

In further embodiments of the processes for converting substrate compound to product compound using the engineered P450-BM3 polypeptides, the suitable reaction conditions can comprise an initial substrate loading to the reaction solution which is then contacted by the polypeptide. This reaction solution is then further supplemented with additional substrate compound as a continuous or batchwise addition over time at a rate of at least about 1 g/L/h, at least about 2 g/L/h, at least about 4 g/L/h, at least about 6 g/L/h, or higher. Thus, according to these suitable reaction conditions, polypeptide is added to a solution having an initial substrate loading of at least about 20 g/L, 30 g/L, or 40 g/L. This addition of polypeptide is then followed by continuous addition of further substrate to the solution at a rate of about 2 g/L/h, 4 g/L/h, or 6 g/L/h until a much higher final substrate loading of at least about 30 g/L, 40 g/L, 50 g/L, 60 g/L, 70 g/L, 100 g/L, 150 g/L, 200 g/L or more, is reached. Accordingly, in some embodiments of the process, the suitable reaction conditions comprise addition of the polypeptide to a solution having an initial substrate loading of at least about 20 g/L, 30 g/L, or 40 g/L followed by addition of further substrate to the solution at a rate of about 2 g/L/h, 4 g/L/h, or 6 g/L/h until a final substrate loading of at least about 30 g/L, 40 g/L, 50 g/L, 60 g/L, 70 g/L, 100 g/L or more, is reached. This substrate supplementation reaction condition allows for higher substrate loadings to be achieved while maintaining high rates of conversion of substrate to product of at least about 50%, 60%, 70%, 80%, 90% or greater conversion of substrate.

In some embodiments, a recycling system is used to recycle NADPH to NADP+. In some embodiments, the recycling system comprises glucose dehydrogenase and glucose. In some embodiments, the recycling system comprises phosphite dehydrogenase and phosphite.

In some embodiments of the processes, the reaction using an engineered P450-BM3 polypeptide comprises the following suitable reaction conditions: (a) substrate loading at about 1-25 g/L; (b) about 1-40 g/L of the engineered polypeptide; (c) 0.25-1 g/L NADP+; (d) a pH of about 7-9; (e) 5%-20% solvent (acetonitrile or DMSO)0% we didn't use a co-solvent; (h) temperature of about 20-30° C.; and (i) reaction time of about 20 hrs.

In some embodiments of the processes, the reaction using an engineered P450-BM3 polypeptide comprises the following suitable reaction conditions: (a) substrate loading at about 10 g/L; (b) about 1.0-5.0 g/L of the engineered polypeptide; (c) 1 g/LNADP+; (d) a pH of about 8; (e) 0% DMSO; (f) temperature of about 30° C.; and (g) reaction time of about 20 hrs.

In some embodiments, additional reaction components or additional techniques are carried out to supplement the reaction conditions. These can include taking measures to stabilize or prevent inactivation of the enzyme, reduce product inhibition, shift reaction equilibrium to product formation.

In further embodiments, any of the above described process for the conversion of substrate compound to product compound can further comprise one or more steps selected from: extraction; isolation; purification; and crystallization of product compound. Methods, techniques, and protocols for extracting, isolating, purifying, and/or crystallizing the product from biocatalytic reaction mixtures produced by the above disclosed processes are known to the ordinary artisan and/or accessed through routine experimentation. Additionally, illustrative methods are provided in the Examples below.

Various features and embodiments of the invention are illustrated in the following representative examples, which are intended to be illustrative, and not limiting.

In light of the guidance provided herein, it is further contemplated that any of the exemplary engineered polypeptides can be used as the starting amino acid sequence for synthesizing other engineered P450-BM3 polypeptides, for example by subsequent rounds of evolution by adding new combinations of various amino acid differences from other polypeptides and other residue positions described herein. Further improvements may be generated by including amino acid differences at residue positions that had been maintained as unchanged throughout earlier rounds of evolution.

### Polynucleotides Encoding Engineered Polypeptides, Expression Vectors and Host Cells:

The present invention provides polynucleotides encoding the engineered P450-BM3 polypeptides described herein. In some embodiments, the polynucleotides are operatively linked to one or more heterologous regulatory sequences that control gene expression to create a recombinant polynucleotide capable of expressing the polypeptide. Expression constructs containing a heterologous polynucleotide encoding the engineered P450-BM3 polypeptides can be introduced into appropriate host cells to express the corresponding P450-BM3 polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the engineered P450-BM3 polypeptide. Thus, having knowledge of a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made encoding the polypeptides described herein by selecting combinations based on the possible codon choices, and all such variations are to be considered specifically disclosed for any polypeptide described herein, including the variants provided in Tables 2.1, 3.1, 4.1, 5.1, 6.1, 7.1, 8.1, 9.1, 10.1, 10.2, 11.1, 11.2, 12.1, 13.1, 14.1, 14.2, 15.1, 16.1, 17.1, 17.2, 18.1, 19.1, and 19.2, as well as SEQ ID NOS: 4, 36, 66, 72, 198, 226, 244, 286, 358, 410, 534, 734, 748, 828, 968, 984, 1160, and 1266.

In various embodiments, the codons are preferably selected to fit the host cell in which the protein is being produced. For example, preferred codons used in bacteria are used for expression in bacteria. Consequently, codon optimized polynucleotides encoding the engineered P450-BM3 polypeptides contain preferred codons at about 40%, 50%, 60%, 70%, 80%, or greater than 90% of codon positions of the full length coding region.

In some embodiments, as described above, the polynucleotide encodes an engineered polypeptide having P450-BM3 activity with the properties disclosed herein, wherein the polypeptide comprises an amino acid sequence having at least 80%, 85%, 86%, 87%, 88%, 89%, 90%, 91%, 92%, 93%, 94%, 95%, 96%, 97%, 98%, 99% or more identity to a reference sequence (e.g., SEQ ID NO: 4, 36, 66, 72, 198, 226, 244, 286, 358, 410, 534, 734, 748, 828, 968, 984, 1160, or 1266), or the amino acid sequence of any variant as disclosed in any of Tables 2.1, 3.1, 4.1, 5.1, 6.1, 7.1, 8.1, 9.1, 10.1, 10.2, 11.1, 11.2, 12.1, 13.1, 14.1, 14.2, 15.1, 16.1, 17.1, 17.2, 18.1, 19.1, and 19.2, and one or more residue differences as compared to the reference polypeptide of SEQ ID NO: 4, 36, 66, 72, 198, 226, 244, 286, 358, 410, 534, 734, 748, 828, 968, 984, 1160, or 1266, or the amino acid sequence of any variant as disclosed in any of Tables 2.1, 3.1, 4.1, 5.1, 6.1, 7.1, 8.1, 9.1, 10.1, 10.2, 11.1, 11.2, 12.1, 13.1, 14.1, 14.2, 15.1, 16.1, 17.1, 17.2, 18.1, 19.1, and 19.2 (for example 1, 2, 3, 4, 5, 6, 7, 8, 9, 10 or more amino acid residue positions). In some embodiments, the reference sequence is selected from SEQ ID NO: 4, 36, 66, 72, 198, 226, 244, 286, 358, 410, 534, 734, 748, 828, 968, 984, 1160, or 1266.

In some embodiments, the polynucleotides are capable of hybridizing under highly stringent conditions to a reference polynucleotide sequence selected from SEQ ID NO: 3, 35, 65, 71, 197, 225, 243, 285, 357, 409, 533, 733, 747, 827, 967, 983, 1159, or 1265, or a complement thereof, or a polynucleotide sequence encoding any of the variant P450-BM3 polypeptides provided herein. In some embodiments, the polynucleotide capable of hybridizing under highly stringent conditions encodes a P450-BM3 polypeptide comprising an amino acid sequence that has one or more residue differences as compared to SEQ ID NO: 4, 36, 66, 72, 198, 226, 244, 286, 358, 410, 534, 734, 748, 828, 968, 984, 1160, or 1266.

In some embodiments, the polynucleotides are capable of hybridizing under highly stringent conditions to a reference polynucleotide sequence selected from any polynucleotide sequence provided herein, or a complement thereof, or a polynucleotide sequence encoding any of the variant enzyme polypeptides provided herein. In some embodiments, the polynucleotide capable of hybridizing under highly stringent conditions encodes an enzyme polypeptide comprising an amino acid sequence that has one or more residue differences as compared to a reference sequence.

In some embodiments, an isolated polynucleotide encoding any of the engineered enzyme polypeptides herein is manipulated in a variety of ways to facilitate expression of the enzyme polypeptide. In some embodiments, the polynucleotides encoding the enzyme polypeptides comprise expression vectors where one or more control sequences is present to regulate the expression of the enzyme polynucleotides and/or polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators. In some embodiments, suitable promoters are selected based on the host cells selection. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xy1A and xy1B genes, and prokaryotic beta-lactamase gene (See e.g., Villa-Kamaroff et al., Proc. Natl Acad. Sci. USA 75: 3727-3731 [1978]), as well as the tac promoter (See e.g., DeBoer et al., Proc. Natl Acad. Sci. USA 80: 21-25 [1983]). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488 [1992]).

In some embodiments, the control sequence is also a suitable transcription terminator sequence (i.e., a sequence recognized by a host cell to terminate transcription). In some embodiments, the terminator sequence is operably linked to the 3′ terminus of the nucleic acid sequence encoding the enzyme polypeptide. Any suitable terminator which is functional in the host cell of choice finds use in the present invention. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra).

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the enzyme polypeptide. Any suitable leader sequence that is functional in the host cell of choice finds use in the present invention. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

In some embodiments, the control sequence is also a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for filamentous fungal host cells include, but are not limited to the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (See e.g., Guo and Sherman, Mol. Cell. Bio., 15:5983-5990 [1995]).

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions that include, but are not limited to those obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 57:109-137 [1993]). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase.

In some embodiments, the control sequence is also a propeptide coding region that codes for an amino acid sequence positioned at the amino terminus of a polypeptide. The resultant polypeptide is referred to as a “proenzyme,” “propolypeptide,” or “zymogen.” A propolypeptide can be converted to a mature active polypeptide by catalytic or autocatalytic cleavage of the propeptide from the propolypeptide. The propeptide coding region may be obtained from any suitable source, including, but not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

In another aspect, the present invention is directed to a recombinant expression vector comprising a polynucleotide encoding an engineered enzyme polypeptide, and one or more expression regulating regions such as a promoter and a terminator, a replication origin, etc., depending on the type of hosts into which they are to be introduced. In some embodiments, the various nucleic acid and control sequences described herein are joined together to produce recombinant expression vectors which include one or more convenient restriction sites to allow for insertion or substitution of the nucleic acid sequence encoding the enzyme polypeptide at such sites. Alternatively, in some embodiments, the nucleic acid sequence of the present invention is expressed by inserting the nucleic acid sequence or a nucleic acid construct comprising the sequence into an appropriate vector for expression. In some embodiments involving the creation of the expression vector, the coding sequence is located in the vector so that the coding sequence is operably linked with the appropriate control sequences for expression.

The recombinant expression vector may be any suitable vector (e.g., a plasmid or virus), that can be conveniently subjected to recombinant DNA procedures and bring about the expression of the enzyme polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present invention provides a host cell comprising at least one polynucleotide encoding at least one engineered enzyme polypeptide of the present invention, the polynucleotide(s) being operatively linked to one or more control sequences for expression of the engineered enzyme enzyme(s) in the host cell. Host cells suitable for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Exemplary host cells also include various Escherichia coli strains (e.g., W3110 (AfhuA) and BL21). Examples of bacterial selectable markers include, but are not limited to the dal genes from Bacillus subtilis or Bacillus lichenformis, or markers, which confer antibiotic resistance such as ampicillin, kanamycin, chloramphenicol, and/or tetracycline resistance.

In some embodiments, the expression vectors of the present invention contain an element(s) that permits integration of the vector into the host cell's genome or autonomous replication of the vector in the cell independent of the genome. In some embodiments involving integration into the host cell genome, the vectors rely on the nucleic acid sequence encoding the polypeptide or any other element of the vector for integration of the vector into the genome by homologous or nonhomologous recombination.

In some alternative embodiments, the expression vectors contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements preferably contain a sufficient number of nucleotides, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A on or the origins of replication of plasmids pBR322, pUC19, pACYC177 (which plasmid has the P15A ori), or pACYC184 permitting replication in E. coli, and pUB110, pE194, or pTA1060 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (See e.g., Ehrlich, Proc. Natl. Acad. Sci. USA 75:1433 [1978]).

In some embodiments, more than one copy of a nucleic acid sequence of the present invention is inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present invention are commercially available. Suitable commercial expression vectors include, but are not limited to the p3xFLAGTM™ expression vectors (Sigma-Aldrich Chemicals), which include a CMV promoter and hGH polyadenylation site for expression in mammalian host cells and a pBR322 origin of replication and ampicillin resistance markers for amplification in E. coli. Other suitable expression vectors include, but are not limited to pBluescriptII SK(−) and pBK-CMV (Stratagene), and plasmids derived from pBR322 (Gibco BRL), pUC (Gibco BRL), pREP4, pCEP4 (Invitrogen) or pPoly (See e.g., Lathe et al., Gene 57:193-201 [1987]).

Thus, in some embodiments, a vector comprising a sequence encoding at least one variant engineered P450-BM3 polypeptide is transformed into a host cell in order to allow propagation of the vector and expression of the variant engineered P450-BM3 polypeptide(s). In some embodiments, the variant engineered P450-BM3 polypeptides are post-translationally modified to remove the signal peptide and, in some cases, may be cleaved after secretion. In some embodiments, the transformed host cell described above is cultured in a suitable nutrient medium under conditions permitting the expression of the variant engineered P450-BM3 polypeptide(s). Any suitable medium useful for culturing the host cells finds use in the present invention, including, but not limited to minimal or complex media containing appropriate supplements. In some embodiments, host cells are grown in HTP media. Suitable media are available from various commercial suppliers or may be prepared according to published recipes (e.g., in catalogues of the American Type Culture Collection).

In another aspect, the present invention provides host cells comprising a polynucleotide encoding an improved engineered P450-BM3 polypeptide provided herein, the polynucleotide being operatively linked to one or more control sequences for expression of the engineered P450-BM3 polypeptide in the host cell. Host cells for use in expressing the engineered P450-BM3 polypeptides encoded by the expression vectors of the present invention are well known in the art and include but are not limited to, bacterial cells, such as E. coli, Bacillus megaterium, Lactobacillus kefir, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Appropriate culture media and growth conditions for the above-described host cells are well known in the art.

Polynucleotides for expression of the engineered P450-BM3 polypeptide may be introduced into cells by various methods known in the art. Techniques include among others, electroporation, biolistic particle bombardment, liposome mediated transfection, calcium chloride transfection, and protoplast fusion. Various methods for introducing polynucleotides into cells are known to those skilled in the art.

In some embodiments, the host cell is a eukaryotic cell. Suitable eukaryotic host cells include, but are not limited to, fungal cells, algal cells, insect cells, and plant cells. Suitable fungal host cells include, but are not limited to, Ascomycota, Basidiomycota, Deuteromycota, Zygomycota, Fungi imperfecti. In some embodiments, the fungal host cells are yeast cells and filamentous fungal cells. The filamentous fungal host cells of the present invention include all filamentous forms of the subdivision Eumycotina and Oomycota. Filamentous fungi are characterized by a vegetative mycelium with a cell wall composed of chitin, cellulose and other complex polysaccharides. The filamentous fungal host cells of the present invention are morphologically distinct from yeast.

In some embodiments of the present invention, the filamentous fungal host cells are of any suitable genus and species, including, but not limited to Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothia, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, and/or Volvariella, and/or teleomorphs, or anamorphs, and synonyms, basionyms, or taxonomic equivalents thereof.

In some embodiments of the present invention, the host cell is a yeast cell, including but not limited to cells of Candida, Hansenula, Saccharomyces, Schizosaccharomyces, Pichia, Kluyveromyces, or Yarrowia species. In some embodiments of the present invention, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments of the invention, the host cell is an algal cell such as Chlamydomonas (e.g., C. reinhardtii) and Phormidium (P. sp. ATCC29409).

In some other embodiments, the host cell is a prokaryotic cell. Suitable prokaryotic cells include, but are not limited to Gram-positive, Gram-negative and Gram-variable bacterial cells. Any suitable bacterial organism finds use in the present invention, including but not limited to Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Campylobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas. In some embodiments, the host cell is a species of Agrobacterium, Acinetobacter, Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, or Zymomonas. In some embodiments, the bacterial host strain is non-pathogenic to humans. In some embodiments the bacterial host strain is an industrial strain. Numerous bacterial industrial strains are known and suitable in the present invention. In some embodiments of the present invention, the bacterial host cell is an Agrobacterium species (e.g., A. radiobacter, A. rhizogenes, and A. rubi). In some embodiments of the present invention, the bacterial host cell is an Arthrobacter species (e.g., A. aurescens, A. citreus, A. globiformis, A. hydrocarboglutamicus, A. mysorens, A. nicotianae, A. paraffineus, A. protophonniae, A. roseoparqffinus, A. sulfureus, and A. ureafaciens). In some embodiments of the present invention, the bacterial host cell is a Bacillus species (e.g., B. thuringensis, B. anthracis, B. megaterium, B. subtilis, B. lentus, B. circulans, B. pumilus, B. lautus, B.coagulans, B. brevis, B. firmus, B. alkaophius, B. licheniformis, B. clausii, B. stearothermophilus, B. halodurans, and B. amyloliquefaciens). In some embodiments, the host cell is an industrial Bacillus strain including but not limited to B. subtilis, B. pumilus, B. licheniformis, B. megaterium, B. clausii, B. stearothermophilus, or B. amyloliquefaciens. In some embodiments, the Bacillus host cells are B. subtilis, B. licheniformis, B. megaterium, B. stearothermophilus, and/or B. amyloliquefaciens. In some embodiments, the bacterial host cell is a Clostridium species (e.g., C. acetobutylicum, C. tetani E88, C. lituseburense, C. saccharobutylicum, C. perfringens, and C. beijerinckii). In some embodiments, the bacterial host cell is a Corynebacterium species (e.g., C. glutamicum and C. acetoacidophilum). In some embodiments the bacterial host cell is an Escherichia species (e.g., E. coli). In some embodiments, the host cell is Escherichia coli W3110. In some embodiments, the bacterial host cell is an Erwinia species (e.g., E. uredovora, E. carotovora, E. ananas, E. herbicola, E. punctata, and E. terreus). In some embodiments, the bacterial host cell is a Pantoea species (e.g., P. citrea, and P. agglomerans). In some embodiments the bacterial host cell is a Pseudomonas species (e.g., P. putida, P. aeruginosa, P. mevalonii, and P. sp. D-01 10). In some embodiments, the bacterial host cell is a Streptococcus species (e.g., S. equisimiles, S. pyogenes, and S. uberis). In some embodiments, the bacterial host cell is a Streptomyces species (e.g., S. ambofaciens, S. achromogenes, S. avermitilis, S. coelicolor, S. aureofaciens, S. aureus, S. fungicidicus, S. griseus, and S. lividans). In some embodiments, the bacterial host cell is a Zymomonas species (e.g., Z. mobilis, and Z. lipolytica).

Many prokaryotic and eukaryotic strains that find use in the present invention are readily available to the public from a number of culture collections such as American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

In some embodiments, host cells are genetically modified to have characteristics that improve protein secretion, protein stability and/or other properties desirable for expression and/or secretion of a protein. Genetic modification can be achieved by genetic engineering techniques and/or classical microbiological techniques (e.g., chemical or UV mutagenesis and subsequent selection). Indeed, in some embodiments, combinations of recombinant modification and classical selection techniques are used to produce the host cells. Using recombinant technology, nucleic acid molecules can be introduced, deleted, inhibited or modified, in a manner that results in increased yields of engineered P450-BM3 variant(s) within the host cell and/or in the culture medium. For example, knockout of Alp1 function results in a cell that is protease deficient, and knockout of pyr5 function results in a cell with a pyrimidine deficient phenotype. In one genetic engineering approach, homologous recombination is used to induce targeted gene modifications by specifically targeting a gene in vivo to suppress expression of the encoded protein. In alternative approaches, siRNA, antisense and/or ribozyme technology find use in inhibiting gene expression. A variety of methods are known in the art for reducing expression of protein in cells, including, but not limited to deletion of all or part of the gene encoding the protein and site-specific mutagenesis to disrupt expression or activity of the gene product. (See e.g., Chaveroche et al., Nucl. Acids Res., 28:22 e97 [2000]; Cho et al., Molec. Plant Microbe Interact., 19:7-15 [2006]; Maruyama and Kitamoto, Biotechnol Lett., 30:1811-1817 [2008]; Takahashi et al., Mol. Gen. Genom., 272: 344-352 [2004]; and You et al., Arch. Microbiol.,191:615-622 [2009], all of which are incorporated by reference herein). Random mutagenesis, followed by screening for desired mutations also finds use (See e.g., Combier et al., FEMS Microbiol. Lett., 220:141-8 [2003]; and Firon et al., Eukary. Cell 2:247-55 [2003], both of which are incorporated by reference).

Introduction of a vector or DNA construct into a host cell can be accomplished using any suitable method known in the art, including but not limited to calcium phosphate transfection, DEAE-dextran mediated transfection, PEG-mediated transformation, electroporation, or other common techniques known in the art. In some embodiments, the Escherichia coli expression vector pCK100900i (See, U.S. Pat. No. 9,714,437, which is hereby incorporated by reference) finds use.

In some embodiments, the engineered host cells (i.e., “recombinant host cells”) of the present invention are cultured in conventional nutrient media modified as appropriate for activating promoters, selecting transformants, or amplifying the engineered P450-BM3 polynucleotide. Culture conditions, such as temperature, pH and the like, are those previously used with the host cell selected for expression, and are well-known to those skilled in the art. As noted, many standard references and texts are available for the culture and production of many cells, including cells of bacterial, plant, animal (especially mammalian) and archaebacterial origin.

In some embodiments, cells expressing the variant engineered P450-BM3 polypeptides of the invention are grown under batch or continuous fermentations conditions. Classical “batch fermentation” is a closed system, wherein the compositions of the medium is set at the beginning of the fermentation and is not subject to artificial alternations during the fermentation. A variation of the batch system is a “fed-batch fermentation” which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. “Continuous fermentation” is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments of the present invention, cell-free transcription/translation systems find use in producing variant engineered P450-BM3 polypeptides(s). Several systems are commercially available, and the methods are well-known to those skilled in the art.

The present invention provides methods of making variant engineered P450-BM3 polypeptides or biologically active fragments thereof. In some embodiments, the method comprises: providing a host cell transformed with a polynucleotide encoding an amino acid sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO: 4, 36, 66, 72, 198, 226, 244, 286, 358, 410, 534, 734, 748, 828, 968, 984, 1160, or 1266, and comprising at least one mutation as provided herein; culturing the transformed host cell in a culture medium under conditions in which the host cell expresses the encoded variant engineered P450-BM3 polypeptide; and optionally recovering or isolating the expressed variant engineered P450-BM3 polypeptide, and/or recovering or isolating the culture medium containing the expressed variant engineered P450-BM3 polypeptide. In some embodiments, the methods further provide optionally lysing the transformed host cells after expressing the encoded engineered P450-BM3 polypeptide and optionally recovering and/or isolating the expressed variant engineered P450-BM3 polypeptide from the cell lysate. The present invention further provides methods of making a variant engineered P450-BM3 polypeptide comprising cultivating a host cell transformed with a variant engineered P450-BM3 polypeptide under conditions suitable for the production of the variant engineered P450-BM3 polypeptides and recovering the engineered P450-BM3 polypeptide. Typically, recovery or isolation of the engineered P450-BM3 polypeptide is from the host cell culture medium, the host cell or both, using protein recovery techniques that are well known in the art, including those described herein. In some embodiments, host cells are harvested by centrifugation, disrupted by physical or chemical means, and the resulting crude extract retained for further purification. Microbial cells employed in expression of proteins can be disrupted by any convenient method, including, but not limited to freeze-thaw cycling, sonication, mechanical disruption, and/or use of cell lysing agents, as well as many other suitable methods well known to those skilled in the art.

Engineered P450-BM3 polypeptides expressed in a host cell can be recovered from the cells and/or the culture medium using any one or more of the techniques known in the art for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B™ (Sigma-Aldrich). Thus, in some embodiments, the resulting polypeptide is recovered/isolated and optionally purified by any of a number of methods known in the art. For example, in some embodiments, the polypeptide is isolated from the nutrient medium by conventional procedures including, but not limited to, centrifugation, filtration, extraction, spray-drying, evaporation, chromatography (e.g., ion exchange, affinity, hydrophobic interaction, chromatofocusing, and size exclusion), or precipitation. In some embodiments, protein refolding steps are used, as desired, in completing the configuration of the mature protein. In addition, in some embodiments, high performance liquid chromatography (HPLC) is employed in the final purification steps. For example, in some embodiments, methods known in the art, find use in the present invention (See e.g., Parry et al., Biochem. J., 353:117 [2001]; and Hong et al., Appl. Microbiol. Biotechnol., 73:1331 [2007], both of which are incorporated herein by reference). Indeed, any suitable purification methods known in the art find use in the present invention.

Chromatographic techniques for isolation of the engineered P450-BM3 polypeptides include, but are not limited to reverse phase chromatography, high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., are known to those skilled in the art.

In some embodiments, affinity techniques find use in isolating the improved engineered P450-BM3 polypeptides. For affinity chromatography purification, any antibody which specifically binds the engineered P450-BM3 polypeptide may be used. For the production of antibodies, various host animals, including but not limited to rabbits, mice, rats, etc., may be immunized by injection with the engineered P450-BM3 polypeptide. The engineered P450-BM3 polypeptide may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (Bacillus Calmette Guerin) and Corynebacterium parvum.

In some embodiments, the engineered P450-BM3 polypeptides are prepared and used in the form of cells expressing the enzymes, as crude extracts, or as isolated or purified preparations. In some embodiments, the engineered P450-BM3 polypeptides are prepared as lyophilisates, in powder form (e.g., acetone powders), or prepared as enzyme solutions. In some embodiments, the engineered P450-BM3 polypeptides are in the form of substantially pure preparations.

In some embodiments, the engineered P450-BM3 polypeptides are attached to any suitable solid substrate. Solid substrates include but are not limited to a solid phase, surface, and/or membrane. Solid supports include, but are not limited to organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of the substrate can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location. A plurality of supports can be configured on an array at various locations, addressable for robotic delivery of reagents, or by detection methods and/or instruments.

In some embodiments, immunological methods are used to purify engineered P450-BM3 polypeptides variants. In one approach, antibody raised against a wild-type or engineered P450-BM3 polypeptide (e.g., against a polypeptide comprising any of SEQ ID NO: 4, 36, 66, 72, 198, 226, 244, 286, 358, 410, 534, 734, 748, 828, 968, 984, 1160, or 1266, and/or a variant thereof, and/or an immunogenic fragment thereof) using conventional methods is immobilized on beads, mixed with cell culture media under conditions in which the variant engineered P450-BM3 is bound, and precipitated. In a related approach, immunochromatography finds use.

In some embodiments, the variant engineered P450-BM3s are expressed as a fusion protein including a non-enzyme portion. In some embodiments, the variant engineered P450-BM3 polypeptide sequence is fused to a purification facilitating domain. As used herein, the term “purification facilitating domain” refers to a domain that mediates purification of the polypeptide to which it is fused. Suitable purification domains include, but are not limited to metal chelating peptides, histidine-tryptophan modules that allow purification on immobilized metals, a sequence which binds glutathione (e.g., GST), a hemagglutinin (HA) tag (corresponding to an epitope derived from the influenza hemagglutinin protein; See e.g., Wilson et al., Cell 37:767 [1984]), maltose binding protein sequences, the FLAG epitope utilized in the FLAGS extension/affinity purification system (e.g., the system available from Immunex Corp), and the like. One expression vector contemplated for use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography; See e.g., Porath et al., Prot. Exp. Purif., 3:263-281 [1992]) while the enterokinase cleavage site provides a means for separating the variant engineered P450-BM3 polypeptide from the fusion protein. pGEX vectors (Promega) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Accordingly, in another aspect, the present invention provides methods of producing the engineered enzyme polypeptides, where the methods comprise culturing a host cell capable of expressing a polynucleotide encoding the engineered enzyme polypeptide under conditions suitable for expression of the polypeptide. In some embodiments, the methods further comprise the steps of isolating and/or purifying the enzyme polypeptides, as described herein.

Appropriate culture media and growth conditions for host cells are well known in the art. It is contemplated that any suitable method for introducing polynucleotides for expression of the enzyme polypeptides into cells will find use in the present invention. Suitable techniques include, but are not limited to electroporation, biolistic particle bombardment, liposome mediated transfection, calcium chloride transfection, and protoplast fusion.

The engineered P450-BM3 with the properties disclosed herein can be obtained by subjecting the polynucleotide encoding the naturally occurring or engineered P450-BM3 polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA 91:10747-10751 [1994]; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 16:258-261 [1998]), mutagenic PCR (See e.g., Caldwell et al., PCR Methods Appl., 3:S136-S140 [1994]), and cassette mutagenesis (See e.g., Black et al., Proc. Natl. Acad. Sci. USA 93:3525-3529 [1996]).

For example, mutagenesis and directed evolution methods can be readily applied to polynucleotides to generate variant libraries that can be expressed, screened, and assayed. Mutagenesis and directed evolution methods are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, 9,665,694, 9,684,771, and all related US, as well as PCT and non-US counterparts; Ling et al., Anal. Biochem., 254(2):157-78 [1997]; Dale et al., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol., 3:284-290 [1999]; Christians et al., Nat. Biotechnol., 17:259-264 [1999]; Crameri et al., Nature, 391:288-291 [1998]; Crameri, et al., Nat. Biotechnol., 15:436-438 [1997]; Zhang et al., Proc. Nat. Acad. Sci. U.S.A., 94:4504-4509 [1997]; Crameri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; and WO 2009/152336, all of which are incorporated herein by reference).

In some embodiments, the enzyme clones obtained following mutagenesis treatment are screened by subjecting the enzymes to a defined temperature (or other assay conditions, such as testing the enzyme's activity on Compound (1)) and measuring the amount of enzyme activity remaining after heat treatments or other assay conditions. Clones containing a polynucleotide encoding a P450-BM3 polypeptide are then sequenced to identify the nucleotide sequence changes (if any), and used to express the enzyme in a host cell. Measuring enzyme activity from the expression libraries can be performed using any suitable method known in the art (e.g., standard biochemistry techniques, such as HPLC analysis).

For engineered polypeptides of known sequence, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides disclosed herein can be prepared by chemical synthesis using the classical phosphoramidite method (See e.g., Beaucage et al., Tetra. Lett., 22:1859-69 [1981]; and Matthes et al., EMBO J., 3:801-05 [1984]), as it is typically practiced in automated synthetic methods. According to the phosphoramidite method, oligonucleotides are synthesized (e.g., in an automatic DNA synthesizer), purified, annealed, ligated and cloned in appropriate vectors.

Accordingly, in some embodiments, a method for preparing the engineered P450-BM3 polypeptide can comprise: (a) synthesizing a polynucleotide encoding a polypeptide comprising greater than 85% identity to an amino acid sequence selected from the amino acid sequence of any variant provided in any of Tables 2.1, 3.1, 4.1, 5.1, 6.1, 7.1, 8.1, 9.1, 10.1, 10.2, 11.1, 11.2, 12.1, 13.1, 14.1, 14.2, 15.1, 16.1, 17.1, 17.2, 18.1, 19.1, and 19.2, as well as SEQ ID NO: 4, 36, 66, 72, 198, 226, 244, 286, 358, 410, 534, 734, 748, 828, 968, 984, 1160, or 1266, and (b) expressing the P450-BM3 polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

The foregoing and other aspects of the invention may be better understood in connection with the following non-limiting examples. The examples are provided for illustrative purposes only and are not intended to limit the scope of the present invention in any way.

### EXPERIMENTAL

The following Examples, including experiments and results achieved, are provided for illustrative purposes only and are not to be construed as limiting the present invention.

In the experimental disclosure below, the following abbreviations apply: ppm (parts per million); M (molar); mM (millimolar), uM and μM (micromolar); nM (nanomolar); mol (moles); gm and g (gram); mg (milligrams); ug and μg (micrograms); L and 1 (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); ° C. (degrees Celsius); CDS (coding sequence); DNA (deoxyribonucleic acid); RNA (ribonucleic acid); NA (nucleic acid; polynucleotide); AA (amino acid; polypeptide); E. coli W3110 (commonly used laboratory E. coli strain, available from the Coli Genetic Stock Center [CGSC], New Haven, CT); HPLC (high pressure liquid chromatography); SDS-PAGE (sodium dodecyl sulfate polyacrylamide gel electrophoresis); PES (polyethersulfone); CFSE (carboxyfluorescein succinimidyl ester); IPTG (isopropyl beta-D-1-thiogalactopyranoside); PMBS (polymyxin B sulfate); NADPH (nicotinamide adenine dinucleotide phosphate); GDH (glucose dehydrogenase); TON (turnover number); FIOPC (fold improvement over positive control); TON (turnover number); ESI (electrospray ionization); LB (Luria broth);TB (terrific broth); MeOH (methanol); Athens Research (Athens Research Technology, Athens, GA); ProSpec (ProSpec Tany Technogene, East Brunswick, NJ); Sigma-Aldrich (Sigma-Aldrich, St. Louis, MO); Ram Scientific (Ram Scientific, Inc., Yonkers, NY); Pall Corp. (Pall, Corp., Pt. Washington, NY); Millipore (Millipore, Corp., Billerica MA); Difco (Difco Laboratories, BD Diagnostic Systems, Detroit, MI); Molecular Devices (Molecular Devices, LLC, Sunnyvale, CA); Kuhner (Adolf Kuhner, AG, Basel, Switzerland); Cambridge Isotope Laboratories, (Cambridge Isotope Laboratories, Inc., Tewksbury, MA); Applied Biosystems (Applied Biosystems, part of Life Technologies, Corp., Grand Island, NY), Agilent (Agilent Technologies, Inc., Santa Clara, CA); Thermo Scientific (part of Thermo Fisher Scientific, Waltham, MA); Fisher (Fisher Scientific, Waltham, MA); Corning (Corning, Inc., Palo Alto, CA); Waters (Waters Corp., Milford, MA); GE Healthcare (GE Healthcare Bio-Sciences, Piscataway, NJ); Pierce (Pierce Biotechnology (now part of Thermo Fisher Scientific), Rockford, IL); Phenomenex (Phenomenex, Inc., Torrance, CA); Optimal (Optimal Biotech Group, Belmont, CA); and Bio-Rad (Bio-Rad Laboratories, Hercules, CA).

### Example 1

Production of Engineered Polypeptides in pCK110900

The polynucleotide (SEQ ID NO: 3) encoding the polypeptide having monooxygenase activity (SEQ ID NO: 4), was cloned into the pCK110900 vector system (See e.g., U.S. Pat. No. 9,714,437, which is hereby incorporated by reference in its entirety) and subsequently expressed in E. coli W3110fhuA under the control of the lac promoter. This polynucleotide, and associated polypeptide, were derived from a previously engineered Bacillus megaterium variant (see U.S. Appln. No. 63/384,746.)

In a 96-well format, single colonies were picked and grown in 190 μL LB media containing 1% glucose and 30 μg/mL CAM at 30° C., 200 rpm, and 85% humidity. Following overnight growth, 20 μL of the grown cultures were transferred into a deep-well plate containing 380 μL of TB media with 30 μg/mL CAM. The cultures were grown at 30° C., 250 rpm, and 85% humidity for approximately 2.5 hours. When the optical density (OD₆₀₀) of the cultures reached 0.4-0.6, expression of the monooxygenase gene was induced by the addition of IPTG to a final concentration of 1 mM. Following induction, growth continued for 18-20 hours at 30° C., 250 rpm with 85% humidity. Cells were harvested by centrifugation at 4,000 rpm and 4° C. for 10 minutes; the supernatant was then discarded. The cell pellets were stored at −80° C. until ready for use.

Prior to performing the assay, the cell pellets were thawed and resuspended in 200 or 300 μL of lysis buffer containing 1 g/L lysozyme, 0.5 g/L PMBS, and 0.025 μL/mL of commercial DNAse (New England BioLabs, M0303L) in 0.1 M potassium phosphate buffer, pH 8.0. The plates were agitated with medium-speed shaking for 2 hours on a microtiter plate shaker at room temperature. The plates were then centrifuged at 4,000 rpm for 10 minutes at 4° C., and the clarified supernatants were used in the HTP assay reaction described in the following examples.

Shake-flask procedures can be used to generate engineered monooxygenase shake-flask powders (SFP), which are useful for secondary screening assays and/or use in the biocatalytic processes described herein. Shake flask powder preparation of enzymes provides a more purified preparation (e.g., up to 30% of total protein) of the engineered enzyme, as compared to the cell lysate used in HTP assays and also allows for the use of more concentrated enzyme solutions. To start the culture, a 10 uL aliquot of a glycerol stock of E. coli containing a plasmid encoding an engineered polypeptide of interest was inoculated into 8 mL of LB cell culture media with 30 μg/mL CAM and 1% glucose. The culture was grown overnight (at least 16 hours) in an incubator at 30° C. with shaking at 250 rpm. The grown culture was then added to 250 mL of TB media with g/mL CAM in a 1-L shake-flask. The 250 mL culture was grown at 30° C. and 250 rpm for 3.5 hours until OD₆₀₀ reached 0.6-0.8. Expression of the monooxygenase gene was induced by the addition of IPTG to a final concentration of 1 mM, and growth was continued for an additional 18-20 hours. Cells were harvested by transferring the culture into a pre-weighed centrifuge bottle which was then centrifuged at 4,000 rpm for 10 minutes at 4° C. The supernatant was discarded, and the remaining cell pellet was weighed. In some embodiments, the cell pellet was stored at −80° C. until ready to use. For lysis, the cell pellet was resuspended in 6 mL/g wet cell weight of 25 mM potassium phosphate at pH 8.0 lysed using a 110L MICROFLUIDIZER® processor system (Microfluidics). Cell debris was removed by centrifugation at 10,000 rpm for 60 minutes at 4° C. The clarified lysate was collected, frozen at −80° C., and then lyophilized, using standard methods known in the art. Lyophilization of frozen clarified lysate provides a dry shake-flask powder comprising crude engineered polypeptide.

### Example 2

Evolution and Screening of Engineered Polypeptides Derived from SEQ ID NO: 4 for Improved Production of Compound (2)

The engineered polynucleotide (SEQ ID NO: 3) encoding the polypeptide with monooxygenase activity of SEQ ID NO: 4 was used to generate the engineered polypeptides of Table 2.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 4, as described below.

Directed evolution began with the polynucleotide set forth in SEQ ID NO: 3. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 75 v/v % of undiluted monooxygenase lysate, prepared as described in EXAMPLE 1, 1 g/L Compound (1), 1 g/L NADPH, 1 g/L GDH-105, 3.6 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 100 μL/well of acetonitrile was added to the reaction plates and mixed well. The plates were sealed and centrifuged at 4,000 rpm for 10 min. An aliquot of the supernatant was removed and further diluted 10-fold into 1:1 acetonitrile:water for achiral LC-MS analysis according to the method in Table 2.2.

TABLE 2.1
Activity of Monooxygenase Variants Relative to SEQ ID NO: 4

| | | FIOP (alcohol |
|---|---|---|
| | | peak area) |
| SEQ ID NO: | Amino Acid Differences (Relative to | Relative to |
| (nt/aa) | SEQ ID NO: 4) | SEQ ID NO: 4 |
| 3/4 | | 1 |
| 5/6 | L83A/F88A/N574T | +++ |
| 7/8 | R32K/L83A/F88A/N574T | +++ |
| 9/10 | L83A/F88A/R111Q/V433D | +++ |
| 11/12 | L83A/F88A/G105L | ++++ |
| 13/14 | R32K/L83A/F88A | ++++ |
| 15/16 | F52Y/L83A/F88A/V433D/N574T | +++ |
| 17/18 | L83A/F88A/T349E | ++++ |
| 19/20 | L83A/F88A/S231R | ++++ |
| 21/22 | F52Y/L83A/F88A/N574T | ++++ |
| 23/24 | R32K/L83A/F88A/V176I | +++ |
| 25/26 | F52Y/L83A/F88A/S231R/V433D/N574T | +++ |
| 27/28 | F52Y/L83A/F88A/V433D | +++ |
| 29/30 | F52Y/L83A/F88A | +++ |
| 31/32 | L83A/F88A | +++ |
| 33/34 | R32K/L83A/F88A/S231R/N574T | +++ |
| 35/36 | F52Y/L83A/F88A/G105V | +++ |
| 37/38 | F52Y/L83A/F88A/S231R | ++++ |
| 39/40 | L83A/F88A/R111Q/N574T | +++ |
| 41/42 | L83A/F88A/R111Q | +++ |
| 43/44 | L83A/F88A/V433D/N574T | +++ |

Levels of increased activity were determined relative to the reference polypeptide of SEQ ID NO: 4 and defined as follows:, “+++” >300, “++++” >500.

TABLE 2.2
Achiral HPLC-MS/MS Method Parameters

| Instrument | | Agilent 1200 series HPLC with a PAL autosampler | | | |
|---|---|---|---|---|---|
| | | and a Sciex 4000 QTRAP mass spectrometer | | | |
| Column | | Agilent Zorbax C18, 150 × 4.6 mm × 5 μm | | | |
| Mobile Phases | | A: 0.1% formic acid in water | | | |
| | | B: 0.1% formic acid in acetonitrile | | | |
| LC Gradient | | 0-0.5 min: 40% B | | | |
| | | 0.51-1.75 min: 100% B | | | |
| | | 1.76-3 min: 40% B | | | |
| Flow rate | | 1 mL/min | | | |
| Run time | | 3 min | | | |
| Column temperature | | 50° C. | | | |
| Injection volume | | 10 μL | | | |
| MS Scan Type | | MRM, positive mode | | | |
| MS Source Temperature | | 450° C. | | | |
| Ionspray voltage | | 5000 | | | |
| Ion Source Gas 1 | | 50 | | | |
| Ion Source Gas 2 | | 50 | | | |
| Declustering potential | | 40 | | | |
| Entrance potential | | 10 | | | |
| Collision cell exit potential | | 15 | | | |
| | Retention | Q1 Mass | Q3 Mass | Collision | Dwell |
| Compound | time | (Da) | (Da) | energy | Time |
| Compound (1) | 2.67 | 230.4 | 84.3 | 30 | 200 msec |
| Compound (2) | 1.73 | 246.4 | 100.4 | 30 | 200 msec |
| Compound (3) | 2.23 | 244.5 | 124.4 | 30 | 200 msec |

### Example 3

Evolution and Screening of Engineered Polypeptides Derived from SEQ ID NO: 36 for Improved Production of Compound (2)

The engineered polynucleotide (SEQ ID NO: 35) encoding the polypeptide with monooxygenase activity of SEQ ID NO: 36 was used to generate the engineered polypeptides of Table 3.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 36, as described below.

Directed evolution began with the polynucleotide set forth in SEQ ID NO: 35. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 5 v/v % of undiluted monooxygenase lysate, prepared as described in EXAMPLE 1, 5 g/L Compound (1), 1 g/L NADPH, 1 g/L GDH-105, 8 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 10-fold into 1:1 acetonitrile:water for achiral LC-MS analysis according to the method in Table 2.2.

TABLE 3.1
Activity of Monooxygenase Variants Relative to SEQ ID NO: 36

| | | FIOP (alcohol |
|---|---|---|
| | | peak area) |
| SEQ ID NO: | Amino Acid Differences (Relative to | Relative to |
| (nt/aa) | SEQ ID NO: 36) | SEQ ID NO: 36 |
| 45/46 | A75S/E374S/Q726L | +++ |
| 47/48 | A75S/Q726L | +++ |
| 49/50 | A75S/G458L/Q726L | ++ |
| 51/52 | A75S/E374S | +++ |
| 53/54 | A75S/G458L | +++ |
| 55/56 | A75S | ++ |
| 57/58 | Q726L | +++ |
| 59/60 | A75S/E374S/G458L/Q726L | +++ |
| 61/62 | E374S/Q726L | +++ |
| 63/64 | R111H/S623Q | ++ |
| 65/66 | R111H/K114G | + |
| 67/68 | R111H/E603F/A604G/S623Q/P853E | ++ |

Levels of increased activity were determined relative to the reference polypeptide of SEQ ID NO: 4 and defined as follows: “+” >0.9, “++” >1.1, “+++” >1.5, “++++” >3.

### Example 4

Evolution and Screening of Engineered Polypeptides Derived from SEQ ID NO: 66 for Improved Production of Compound (2)

The engineered polynucleotide (SEQ ID NO: 65) encoding the polypeptide with monooxygenase activity of SEQ ID NO: 66 was used to generate the engineered polypeptides of Table 4.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 66, as described below.

Directed evolution began with the polynucleotide set forth in SEQ ID NO: 65. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 5 v/v % of undiluted monooxygenase lysate, prepared as described in EXAMPLE 1, 5 g/L Compound (1), 1 g/L NADPH, 1 g/L GDH-105, 8 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 10-fold into 1:1 acetonitrile:water for achiral LC-MS analysis according to the method in Table 2.2.

TABLE 4.1
Activity of Monooxygenase Variants Relative to SEQ ID NO: 66

| | Amino Acid Differences | Alcohol FIOP |
|---|---|---|
| SEQ ID NO: | (Relative to SEQ ID | Relative to |
| (nt/aa) | NO: 66) | SEQ ID NO: 66 |
| 69/70 | T328V | +++ |
| 71/72 | L438R | ++++ |
| 73/74 | L182V | ++ |
| 75/76 | L189T | ++ |
| 77/78 | L182I | ++ |
| 79/80 | M186R | +++ |
| 81/82 | A331F | +++ |
| 83/84 | M355V | + |
| 85/86 | H267T | +++ |
| 87/88 | A75K | +++ |
| 89/90 | T328S | ++ |
| 91/92 | A331T | +++ |
| 93/94 | L189G | + |
| 95/96 | A181V | +++ |
| 97/98 | A331M | +++ |
| 99/100 | M238L | +++ |
| 101/102 | Q74G | ++ |
| 103/104 | I358T | ++ |
| 105/106 | V179M | ++ |
| 107/108 | T437S | +++ |
| 109/110 | A83V | ++ |
| 111/112 | T437G | +++ |
| 113/114 | I358V | +++ |
| 115/116 | V179R | +++ |
| 117/118 | E268Q | ++++ |
| 119/120 | A75T | ++ |

Levels of increased activity were determined relative to the reference polypeptide of SEQ ID NO: 66 and defined as follows: “+” >1, “++” >1.1, “+++” >1.5, “++++” >3.

### Example 5

Evolution and Screening of Engineered Polypeptides Derived from SEQ ID NO: 72 for Improved Production of Compounds (2) and (3)

The engineered polynucleotide (SEQ ID NO: 71) encoding the polypeptide with monooxygenase activity of SEQ ID NO: 72 was used to generate the engineered polypeptides of Table 5.1. These polypeptides displayed improved monooxygenase activity under the desired conditions e.g., the improvement in the formation of the alcohol Compound (2) and/or ketone compound (3), from the substrate Compound (1), as compared to the starting polypeptide. The engineered polypeptides, having the amino acid sequences of even-numbered sequence identifiers were generated from the “backbone” amino acid sequence of SEQ ID NO: 72, as described below.

Directed evolution began with the polynucleotide set forth in SEQ ID NO: 71. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2) or (3).

The enzyme assays were carried out in 96-well deep-well (1.1 mL total volume) plates, in 100 μL total reaction volume per well. The reactions contained 2 v/v % of undiluted monooxygenase lysate, prepared as described in EXAMPLE 1, 5 g/L Compound (1), 1 g/L NADPH, 0.1 g/L GDH-105, 8 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 40-fold into 1:1 acetonitrile:water for achiral LC-MS analysis.

TABLE 5.1
Activity of Monooxygenase Variants Relative to SEQ ID NO: 72

| | | | Alcohol |
|---|---|---|---|
| | | Ketone peak | peak area |
| | | area FIOP | FIOP |
| SEQ ID | | Relative to | Relative to |
| NO: | Amino Acid Differences | SEQ ID | SEQ ID |
| (nt/aa) | (Relative to SEQ ID NO: 72) | NO: 72 | NO: 72 |
| 121/122 | A83V/E268Q/T328S/A331M/ | ++ | |
| | M355V/I358V | | |
| 123/124 | A83V/L182I/E268Q/T328V/ | ++ | |
| | M355V/I358V/T437S | | |
| 125/126 | A83V/L189G/E268Q/T328V/ | ++ | +++ |
| | M355V/I358V/T437G | | |
| 127/128 | Q74G/H267T/E268Q/T328V | ++ | ++ |
| 129/130 | A83V/L182I/L189G/T328V/ | ++ | ++ |
| | A331M | | |
| 131/132 | A75T/A83V/L189G/H267T/E268Q | ++ | +++ |
| 133/134 | L189G/H267T/E268Q/T437S | ++ | +++ |
| 135/136 | A83V/V179R/T328V/A331M | +++ | ++++ |
| 137/138 | A83V/E268Q/T328S/A331M/ | +++ | +++ |
| | I358T/T437G | | |
| 139/140 | E268Q/I358V | +++ | + |
| 141/142 | E268Q/T328V/A331M | +++ | |
| 143/144 | T328V/A331M/I358T | ++ | +++ |
| 145/146 | L189G/E268Q/I358V | +++ | ++ |
| 147/148 | A83V/E268Q/A331T | +++ | +++ |
| 149/150 | A83V/V179R/M355V/I358V/T437G | ++ | +++ |
| 151/152 | Q74G/A75T/A83V/V179R/L189G/ | ++ | |
| | T328V/A331T/T437G | | |
| 153/154 | A75T/A83V/H267T/E268Q/M355V/ | +++ | ++++ |
| | I358V/M654L | | |
| 155/156 | Q74G/A75T/E268Q/T328V/A331T/ | +++ | +++ |
| | I358V/T437S | | |
| 157/158 | A83V/A331T/T437S | ++ | +++ |
| 159/160 | Q74G/A83V/E268Q/T328S/A331M/ | +++ | +++ |
| | I358V/T437G | | |
| 161/162 | A83V/E268Q | +++ | |
| 163/164 | V179R/L189G/A331M/T437S | ++ | +++ |
| 165/166 | V179R/L182I/E268Q | +++ | |
| 167/168 | L189G/E268Q/T328V/A331T/I358V/ | +++ | ++++ |
| | T437S | | |
| 169/170 | A83V/V179R/L182V/T437S | ++ | ++ |
| 171/172 | A83V/V179R/L189G/A331M/ | ++ | +++ |
| | M355V | | |
| 173/174 | A75T/A83V/V179R/L189G/A331M | ++ | +++ |
| 175/176 | A83V/L182I/L189G/E268Q/T328V/ | ++ | +++ |
| | A331M/M355V/I358V/T437G | | |
| 177/178 | A83V/A331M | ++ | +++ |
| 179/180 | A75T/E268Q/T328V/A331T/I358T | ++++ | +++ |
| 181/182 | L189G/E268Q/T328V | ++ | + |
| 183/184 | A83V/H267T/E268Q/T328V/ | +++ | +++ |
| | A331M/M355V/I358T | | |
| 185/186 | H267T/E268Q/A331T/M355V/ | +++ | +++ |
| | I358T/T437S | | |
| 187/188 | A83V/I358V/T437S | ++ | ++ |
| 189/190 | A83V/L189G/T328V/A331M/T437G | ++ | + |
| 191/192 | E268Q/T328V/M355V/I358T | +++ | ++++ |
| 193/194 | A83V/E268Q/T328V/A331M | +++ | +++ |
| 195/196 | A75T/A83V/V179R/E268Q/T328V/ | +++ | +++ |
| | A331M/T437G | | |
| 197/198 | A83V/E268Q/T328V/A331M/I358V | ++++ | ++++ |
| 199/200 | A83V/L189G/E268Q/T328V/A331M | +++ | +++ |
| 201/202 | V179R/A331M/I358V | ++ | +++ |
| 203/204 | E268Q/A331M/M355V/I358V | +++ | +++ |
| 205/206 | A83V/E268Q/T328S/A331T/T437G | +++ | +++ |
| 207/208 | A83V/L189G/H267T/E268Q/I358T | +++ | +++ |
| 209/210 | E268Q/M355V/I358V/T437G | ++ | +++ |
| 211/212 | H267T/E268Q/T328V | ++ | ++ |
| 213/214 | E268Q | ++ | |
| 215/216 | A75T/A83V/E268Q/T437G | ++ | ++ |
| 217/218 | H267T/E268Q/T328V/A331M/ | ++ | +++ |
| | M355V/I358T | | |
| 219/220 | V179R/T328V/A331M | ++ | ++++ |

Levels of increased activity for ketone peak area FIOP were determined relative to the reference polypeptide of SEQ ID NO: 72 and defined as follows: “+” >1.5, “++” >5, “+++” >10, “++++” >50.
Levels of increased activity for alcohol peak area FIOP were determined relative to the reference polypeptide of SEQ ID NO: 72 and defined as follows: “+” >1, “++” >1.15, “+++” >1.5, “++++” >3.

### Example 6

Evolution and Screening of Engineered Polypeptides Derived from SEQ ID NO: 198 for Improved Production of Compound (2)

The engineered polynucleotide (SEQ ID NO: 197) encoding the polypeptide with monooxygenase activity of SEQ ID NO: 198 was used to generate the engineered polypeptides of Table 6.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 198, as described below.

Directed evolution began with the polynucleotide set forth in SEQ ID NO: 197. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 35 v/v % of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 200 μL/well of acetonitrile was added to the reaction plates and mixed well. The plates were sealed and centrifuged at 4,000 rpm for 10 min. An aliquot of the supernatant was removed and further diluted 80-fold into 1:1 acetonitrile:water for achiral LC-MS analysis according to the method in Table 2.2.

TABLE 6.1
Activity of Monooxygenase Variants Relative to SEQ ID NO: 198

| SEQ ID NO: | Amino Acid Differences | alcohol Relative |
|---|---|---|
| (nt/aa) | (Relative to SEQ ID NO: 198) | to SEQ ID NO: 198 |
| 221/222 | A79I | + |
| 223/224 | M213V | + |
| 225/226 | Y257Q | + |

Levels of increased activity were determined relative to the reference polypeptide of SEQ ID NO: 198 and defined as follows: “+” >0.9, “++” >1.10.

### Example 7

Evolution and Screening of Engineered Polypeptides Derived from SEQ ID NO: 226 for Improved Production of Compound (2)

The engineered polynucleotide (SEQ ID NO: 225) encoding the polypeptide with monooxygenase activity of SEQ ID NO: 226 was used to generate the engineered polypeptides of Table 7.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 226, as described below.

Directed evolution began with the polynucleotide set forth in SEQ ID NO: 225. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 5 v/v % of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 80-fold into 1:1 acetonitrile:water for achiral LC-MS analysis according to the method in Table 2.2.

TABLE 7.1
Activity of Monooxygenase Variants Relative to SEQ ID NO: 226

| | | Alcohol FIOP |
|---|---|---|
| SEQ ID NO: | Amino Acid Differences (Relative | Relative to SEQ |
| (nt/aa) | to SEQ ID NO: 226) | ID NO: 226 |
| 227/228 | A502T | ++ |
| 229/230 | A385R | + |
| 231/232 | Q547V | + |
| 233/234 | V315M | + |
| 235/236 | D503G | + |
| 237/238 | V315T | +++ |
| 239/240 | A576P | + |
| 241/242 | N320F | + |
| 243/244 | A577G | +++ |
| 245/246 | A576V | +++ |
| 247/248 | I504R | ++ |
| 249/250 | Q405A | + |
| 251/252 | Q398V | ++ |
| 253/254 | P542T | + |
| 255/256 | I504T | ++ |
| 257/258 | F391Y | ++ |
| 259/260 | P541A | + |
| 261/262 | Q388L | ++ |
| 263/264 | K573S | ++ |
| 265/266 | T493W | + |
| 267/268 | T497A | + |
| 269/270 | I504W | +++ |

Levels of increased activity were determined relative to the reference polypeptide of SEQ ID NO: 226 and defined as follows: “+” >1.5, “++” >2, “+++” >3.

### Example 8

Evolution and Screening of Engineered Polypeptides Derived from SEQ ID NO: 244 for Improved Production of Compound (2)

The engineered polynucleotide (SEQ ID NO: 243) encoding the polypeptide with monooxygenase activity of SEQ ID NO: 244 was used to generate the engineered polypeptides of Table 8.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 244, as described below.

Directed evolution began with the polynucleotide set forth in SEQ ID NO: 243. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 5 v/v % of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 80-fold into 1:1 acetonitrile:water for achiral LC-MS analysis according to the method in Table 2.2.

Select variants were additionally analyzed for selectivity. The enzyme assays were carried out in 96-well deep-well (1.1 mL total volume) plates, in 100 μL total reaction volume per well. The reactions contained 40 v/v % of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 50 μL/well of a 25 uL/mL solution triethylamine in acetonitrile was added to the reaction plates, followed by 50 uL/well of 37 g/L solution 2-bromoacetophenone in acetonitrile. The plates were heat-sealed and shaken for 2 hours at 50° C. After 2 hours the plates were centrifuged for 10 minutes at 4,000 rpm. Then, the supernatant was filtered through a hydrophilic filter plate. An aliquot of the filtrate was removed and further diluted 10-fold into 1:1 acetonitrile:water for chiral LC-MS analysis according to the method described in Table 8.2.

TABLE 8.1
Activity of Monooxygenase Variants Relative to SEQ ID NO: 244

| | | % conversion | |
|---|---|---|---|
| | | to alcohol | |
| | | FIOP | |
| SEQ ID | | Relative to | % ee |
| NO: | Amino Acid Differences | SEQ ID | (alcohol, |
| (nt/aa) | (Relative to SEQ ID NO: 244) | NO: 244 | LCMS) |
| 271/272 | A75S/M178V/M213V/V315T | + | |
| 273/274 | K573S | ++ | + |
| 275/276 | M178V | +++ | |
| 277/278 | M178V/A576V | ++ | |
| 279/280 | M178V/M213V/K573S | ++ | |
| 281/282 | M178V/M213V/Q726L | ++ | |
| 283/284 | M178V/Q726L | ++++ | |
| 285/286 | M178V/T437S | ++ | + |
| 287/288 | M178V/T497A/Q726L | +++ | |
| 289/290 | M178V/V179R/A576V | ++ | + |
| 291/292 | M178V/V179R/K573S/Q726L | +++ | |
| 293/294 | M178V/V179R/M213V/T437S/ | ++ | |
| | T497A/K573S/A576V | | |
| 295/296 | M331T | ++ | |
| 297/298 | M331T/K573S/A576V | ++ | |
| 299/300 | M331T/T497A | ++ | |
| 301/302 | M331T/V358T/F391Y/T437S | ++ | |
| 303/304 | Q726L | ++ | |
| 305/306 | T497A/K573S | ++ | |
| 307/308 | V179R/Q726L | ++ | |
| 309/310 | V179R/V358T | ++ | |
| 311/312 | A75S/M331T/A576V/Q726L | + | + |
| 313/314 | L685F | + | |
| 315/316 | L756Q | + | |
| 317/318 | I999A | + | |
| 319/320 | F889Q | + | |
| 321/322 | Y708C | ++ | |
| 323/324 | T682K | ++ | |
| 325/326 | K797H | ++ | |
| 327/328 | E888Y | + | |
| 329/330 | V794C | + | |
| 331/332 | P759T | ++ | |
| 333/334 | S848T | + | |
| 335/336 | S851G | + | |
| 337/338 | G1003W | ++ | |
| 339/340 | D699R | ++ | |
| 341/342 | A796H | ++ | |
| 343/344 | I704L | ++ | |
| 345/346 | K862L | ++ | |
| 347/348 | L701V | + | |
| 349/350 | A796N | + | |
| 351/352 | N707Y | + | |
| 353/354 | S851V | + | |
| 355/356 | A1048L | + | |

Levels of increased activity were determined relative to the reference polypeptide of SEQ ID NO: 244 and defined as follows: “+” >1, “++” >1.15, “+++” >1.5, “++++” >2. Levels of enantiomeric excess are measured as % ee and defined as follows: “, “+” >20% SEQ ID NO: 244 has 16% ee as reference.

Derivatization was performed to facilitate chiral LC-MS analysis, as depicted below in Scheme 5.

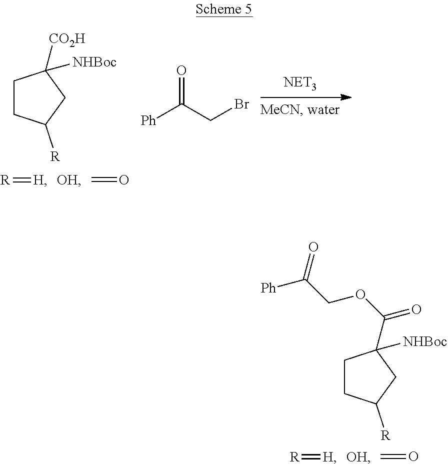

TABLE 8.2
Chiral HPLC-MS/MS Method Parameters

| Instrument | Waters Acquity UPLC system equipped with a binary |
|---|---|
| | pump and TUV UV detector and 3200 Sciex QTrap Mass |
| | Spectrometer with TurbeV ion source |
| Column | OJ-3R, 4.6 × 150 mm, 3 μm |
| Mobile Phases | A: 0.1% formic acid in water |
| | B: 0.1% formic acid in acetonitrile |
| LC Gradient | 0-2 min: 20% B |
| | 2-9.4 min: 20 to 50% B |
| | 9.4-9.5 min: 50 to 100% B |
| | 9.5-11 min: 100% B |
| | 11-11.1 min: 100% B to 20% B |
| | 11.1-12 min: 20% B |
| Flow rate | 1.2 mL/min |
| Run time | 12 min |
| Column temperature | 30° C. |
| Injection volume | 10 μL |
| MS Scan Type | MRM, positive mode |
| MS Source Temperature | 550° C. |
| Ionspray voltage | 5500 |
| Ion Source Gas 1 | 50 |
| Ion Source Gas 2 | 30 |
| Declustering potential | 40 |
| Entrance potential | 10 |
| Collision cell exit potential | 3 |

| | | Q1 Mass | Q3 Mass | Collision | Dwell |
|---|---|---|---|---|---|
| Compound | Retention time | (Da) | (Da) | energy | Time |
| Compound (2) | 8.2 and 8.3 for | 364.2 | 99.8 | 30 | 200 msec |
| | desired isomers, | | | | |
| | 7.9 and 9.1 for | | | | |
| | undesired isomers | | | | |

### Example 9

Evolution and Screening of Engineered Polypeptides Derived from SEQ ID NO: 286 for Improved Production of Compound (2)

The engineered polynucleotide (SEQ ID NO: 285) encoding the polypeptide with monooxygenase activity of SEQ ID NO: 286 was used to generate the engineered polypeptides of Table 9.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 286, as described below.

Directed evolution began with the polynucleotide set forth in SEQ ID NO: 285. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 5 v/v % of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 80-fold into 1:1 acetonitrile:water for achiral LC-MS analysis according to the method described in Table 2.2.

Select variants were additionally analyzed for selectivity. The enzyme assays were carried out in 96-well deep-well (1.1 mL total volume) plates, in 100 μL total reaction volume per well. The reactions contained 40 v/v % of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 50 μL/well of a 25 uL/mL solution triethylamine in acetonitrile was added to the reaction plates, followed by 50 uL/well of 37 g/L solution 2-bromoacetophenone in acetonitrile. The plates were heat-sealed and shaken for 2 hours at 50° C. After 2 hours the plates were centrifuged for 10 minutes at 4,000 rpm. Then, the supernatant was filtered through a hydrophilic filter plate. An aliquot of the filtrate was removed and further diluted 10-fold into 1:1 acetonitrile:water for chiral LC-MS analysis according to the method described in Table 8.2.

TABLE 9.1
Activity of Monooxygenase Variants Relative to SEQ ID NO: 286

| | | FIOP, | | |
|---|---|---|---|---|
| | | activity | | |
| SEQ ID | | Relative to | | |
| NO: | Amino Acid Differences | SEQ ID | alcohol | ketone |
| (nt/aa) | (Relative to SEQ ID NO: 286) | NO: 286 | % ee | % ee |
| 357/358 | A88S/G522R | | ++ | +++ |
| 359/360 | L87V | | + | ++ |
| 361/362 | M331S | | + | |
| 363/364 | T269P | | + | +++ |
| 365/366 | T269G | | + | ++ |
| 367/368 | L87I | | + | ++ |
| 369/370 | Q405A | + | | |
| 371/372 | P330G | | + | |
| 373/374 | L234F | + | | |
| 375/376 | L408R | + | | |
| 377/378 | T89S | ++ | | |
| 379/380 | Q405S | ++ | | |
| 381/382 | Q398R | + | | |
| 383/384 | A411G | + | | |
| 385/386 | V328T | + | | |
| 387/388 | A75S/Q726L/A796N/S851V/ | + | | |
| | I999A | | | |
| 389/390 | A75S/M331T/Q726L/I999A | + | | |
| 391/392 | A75S/M331T/L701V/Q726L/ | + | | |
| | S851V/A1048L | | | |
| 393/394 | A75S/A1048L | + | | |

Levels of increased activity were determined relative to the reference polypeptide of SEQ ID NO: 286 and defined as follows: “+” >1.2, “++” >2. Levels of enantiomeric excess are measured as % ee and defined as follows: “+” >50%, “++” >78%, “+++” >90%. SEQ ID 286 has 38% ee for the alcohol and 78% ee for the ketone.

### Example 10

Evolution and Screening of Engineered Polypeptides Derived from SEQ ID NO: 358 for Improved Production of Compound (2)

The engineered polynucleotide (SEQ ID NO: 357) encoding the polypeptide with monooxygenase activity of SEQ ID NO: 358 was used to generate the engineered polypeptides of Tables 10.1 and 10.2. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 358, as described below.

Directed evolution began with the polynucleotide set forth in SEQ ID NO: 357. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 5 v/v % of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 80-fold into 1:1 acetonitrile:water for achiral LC-MS analysis according to the method described in Table 2.2.

Select variants were additionally analyzed for selectivity. The enzyme assays were carried out in 96-well deep-well (1.1 mL total volume) plates, in 100 μL total reaction volume per well. The reactions contained 40 v/v % of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 50 μL/well of a 25 uL/mL solution triethylamine in acetonitrile was added to the reaction plates, followed by 50 uL/well of 37 g/L solution 2-bromoacetophenone in acetonitrile. The plates were heat-sealed and shaken for 2 hours at 50° C. After 2 hours the plates were centrifuged for 10 minutes at 4,000 rpm. Then, the supernatant was filtered through a hydrophilic filter plate. An aliquot of the filtrate was removed and further diluted 10-fold into 1:1 acetonitrile:water for chiral LC-MS analysis according to the method described in Table 8.2.

TABLE 10.1
Activity of Monooxygenase Variants Relative to SEQ ID NO: 358

| | | FIOP (apparent % | |
|---|---|---|---|
| | | conversion to | |
| SEQ ID | | alcohol) Relative | |
| NO: | Amino Acid Differences | to SEQ ID | % ee |
| (nt/aa) | (Relative to SEQ ID NO: 358) | NO: 358 | (alcohol) |
| 395/396 | T269P | | ++ |
| 397/398 | R522G/Q726L | ++ | |
| 399/400 | Q726L/A1048L | +++ | + |
| 401/402 | T269P/R522G/N707Y | ++ | ++ |
| 403/404 | A75S/T269P/N707Y | + | ++ |
| 405/406 | R522G | +++ | |
| 407/408 | T269P/R522G/N707Y/A1048L | | ++ |
| 409/410 | R522G/A1048L | +++ | |

Levels of increased activity were determined relative to the reference polypeptide of SEQ ID NO: 358 and defined as follows: “+” >1 “++” >1.15, “+++” >1.5. Levels of enantiomeric excess are measured as % ee and defined as follows: >80%, “++” >90%. SEQ ID NO: 358 has 80% ee.

Analysis of other variants derived from SEQ ID NO: 357 was carried out in 96-well deep-well (1.1 mL total volume) plates, in 100 μL total reaction volume per well. The reactions contained 10 v/v % of undiluted monooxygenase lysate, prepared as described in EXAMPLE 1 and then heated to 40° C. for 2 hours, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 80-fold into 1:1 acetonitrile:water for achiral LC-MS analysis.

TABLE 10.2
Activity of Monooxygenase Variants Relative to SEQ ID NO: 358

| | | FIOP (apparent % |
|---|---|---|
| SEQ ID | | conversion to |
| NO: | Amino Acid Differences | alcohol) Relative to |
| (nt/aa) | (Relative to SEQ ID NO: 358) | SEQ ID NO: 358 |
| 411/412 | E1038Q | ++ |
| 413/414 | Q993S | + |
| 415/416 | E692G | + |
| 417/418 | Q924N | + |
| 419/420 | Q646F | ++ |
| 421/422 | Q788P | + |
| 423/424 | Q170T | +++ |
| 425/426 | P676R | ++ |
| 427/428 | K814I | ++ |
| 429/430 | P969E | ++++ |
| 431/432 | E888G | ++++ |
| 433/434 | E692V | ++++ |
| 435/436 | Q170S | ++++ |
| 437/438 | E786V | ++ |
| 439/440 | E786S | ++ |
| 441/442 | D876G | ++ |
| 443/444 | K814S | ++ |
| 445/446 | N630G | ++ |
| 447/448 | P676K | ++ |
| 449/450 | Q547R | ++ |
| 451/452 | P462W | ++ |
| 453/454 | E786G | ++ |
| 455/456 | A651S | ++ |
| 457/458 | E672G | ++ |
| 459/460 | K77L | ++ |
| 461/462 | K787R | +++ |
| 463/464 | A557P | ++ |
| 465/466 | K973P | + |
| 467/468 | E989G | ++ |
| 469/470 | Q924G | ++ |
| 471/472 | K893G | ++ |
| 473/474 | A557E | ++ |
| 475/476 | S955G | + |
| 477/478 | E786R | ++ |
| 479/480 | P969G | ++ |
| 481/482 | T877V | ++ |
| 483/484 | H941R | ++ |
| 485/486 | Q924P | ++ |
| 487/488 | K841R | + |
| 489/490 | Q289A | + |
| 491/492 | H286R | ++ |
| 493/494 | Q982G | ++ |
| 495/496 | E896L | ++ |
| 497/498 | E989L | +++ |
| 499/500 | P775K | +++ |
| 501/502 | Q924A | +++ |

Levels of increased activity were determined relative to the reference polypeptide of SEQ ID NO: 358 and defined as follows: “+” >1.2, “++” >1.5, “+++” >3, “++++” >5.

### Example 11

Evolution and Screening of Engineered Polypeptides Derived from SEQ ID NO: 410 for Improved Production of Compound (2)

The engineered polynucleotide (SEQ ID NO: 409) encoding the polypeptide with monooxygenase activity of SEQ ID NO: 410 was used to generate the engineered polypeptides of Tables 11.1 and 11.2. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 410, as described below.

Directed evolution began with the polynucleotide set forth in SEQ ID NO: 409. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 10 v/v % of undiluted monooxygenase lysate, prepared as described in EXAMPLE 1 and then heated to 42.5° C. for 2 hours, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours. Some variants were also analyzed under similar conditions with 5% v/v of undiluted monooxygenase lysate, prepared as described in EXAMPLE 1 and not subject to thermal pre-incubation

After overnight incubation, 400 μL/well of acetonitrile was added to the reaction plates and mixed well. The plates were sealed and centrifuged at 4,000 rpm for 10 min. An aliquot of the supernatant was removed and further diluted 400 or 800-fold into 1:1 acetonitrile:water for achiral LC-MS analysis according to the conditions in Table 2.2.

TABLE 11.1
Activity of Monooxygenase Variants Relative to SEQ ID NO: 410

| | | FIOP | |
|---|---|---|---|
| | | (apparent | |
| | | % | |
| | | conversion | FIOP |
| | | to alcohol, | without |
| | | with | heat |
| | | thermostability | treatment |
| | | challenge at | Relative |
| | | 42.5° C.) | to |
| SEQ ID | | Relative to | SEQ ID |
| NO: | | SEQ ID | NO: |
| (nt/aa) | Amino Acid Differences (Relative to SEQ ID NO: 410) | NO: 410 | 410 |
| 503/504 | Q170T/E410S/A557E/E786V/K787R/K814S/Q924G/E989G | +++ | |
| 505/506 | G158L/Q170T | +++ | |
| 507/508 | G158L/E410S/P462W/A557E/P969G | ++++ | |
| 509/510 | G158L/Q170T/E410S/P462W/Q726L/E786G/K814S | ++++ | |
| 511/512 | G158L/E410S/A557P/E786G/K787R | +++ | |
| 513/514 | P462W/A557P/Q726L/E786G/K787R/Q924G | +++ | |
| 515/516 | G158L/E410S/K862L | ++++ | |
| 517/518 | G158L/E410S/K814S/Q924G | ++++ | |
| 519/520 | G158L/A557P/Q726L/E786G/Q788P/K862L | +++ | |
| 521/522 | G158L/A557P/N630G/K814S | +++ | |
| 523/524 | G158L/K814S/Q924G | +++ | |
| 525/526 | G158L/P462W/N630G/E786R/Q788P/P969G | +++ | |
| 527/528 | G158L/A557P/E786V | +++ | |
| 529/530 | G158L/Q726L | +++ | |
| 531/532 | G158L/Q170T/E410S/Q924G | ++++ | |
| 533/534 | G158L/A557P | +++ | |
| 535/536 | G158L/N630G/E786S/K814S | +++ | |
| 537/538 | G158L/A557P/E786R/K787R/Q788P | +++ | |
| 539/540 | G158L/P462W/A557P/N630G/Q924G | +++ | |
| 541/542 | G158L/A557P/K814S/Q924G | +++ | |
| 543/544 | G158L/Q170T/N630G/Q726L/E786G/Q788P/K814S | ++++ | |
| 545/546 | E410S/P462W/A557P/N630G/E786V/K787R/P969G | ++ | |
| 547/548 | G158L/E410S | ++++ | |
| 549/550 | G158L/Q170T/E410S/P462W/N630G/E672G/Q726L/E786R/Q788P/ | ++ | |
| | K814S/Q924G | | |
| 551/552 | G158L/E786S/K787R/Q788P | +++ | |
| 553/554 | A385R | ++ | |
| 555/556 | A652S | ++ | |
| 557/558 | A717C | | ++ |
| 559/560 | A717Q | ++ | ++ |
| 561/562 | D553A | ++ | + |
| 563/564 | D553G | ++ | + |
| 565/566 | D553S | ++ | + |
| 567/568 | E871N | ++ | |
| 569/570 | E923A | ++ | + |
| 571/572 | E923L | ++ | |
| 573/574 | K469R | ++ | + |
| 575/576 | K640C | ++ | |
| 577/578 | K640G | ++ | ++ |
| 579/580 | K640V | ++ | ++ |
| 581/582 | K640W | ++ | ++ |
| 583/584 | L645V | | ++ |
| 585/586 | L795A | ++ | ++ |
| 587/588 | N574H | ++ | |
| 589/590 | N574S | | ++ |
| 591/592 | Q926K | + | ++ |
| 593/594 | Q926R | ++ | |
| 595/596 | S556G | ++ | ++ |
| 597/598 | S650P | ++ | |
| 599/600 | V773A | ++ | |
| 601/602 | V773G | ++ | |
| 603/604 | V779G | ++ | |
| 605/606 | V838W | ++ | |
| 607/608 | A385T | ++ | |
| 609/610 | N523R | ++ | |
| 611/612 | D553K | ++ | + |
| 613/614 | D550T | ++ | ++ |
| 615/616 | E871M | ++ | + |
| 617/618 | T613A | ++ | + |

Levels of increased activity for the thermostability challenge were determined relative to the reference polypeptide of SEQ ID NO: 410 and defined as follows: “+” > 1 “++” > 1.15, “+++” > 100, “++++” > 300.
Levels of increased activity (without heat treatment) were determined relative to the reference polypeptide of SEQ ID NO: 410 and defined as follows: “+” > 1 “++” > 1.15, “+++” > 1.5

Other variants derived from SEQ ID NO: 409 were carried out in 96-well deep-well (1.1 mL total volume) plates, in 100 μL total reaction volume per well. The reactions contained 5 v/v % of undiluted monooxygenase lysate, prepared as described in EXAMPLE 1, 10 g/L Compound (1), 1 g/L NADPH, 0.25 g/L PDH-102, and dissolved in 285 mM sodium phosphite buffer, pH 8. The reaction plates were sealed with an aeropore seal and shaken at 600 rpm at 30° C. with 85% humidity for 18 hours.

These same variants were analyzed with lysate that had been heat-treated. The reactions contained 40 v/v % of monooxygenase lysate, prepared as described in EXAMPLE 1, then diluted 4-fold into 300 mM pH 8 sodium phosphite and heated to 42.5° C. for two hours, 10 g/L Compound (1), 1 g/L NADPH, 0.25 g/L PDH-102, and dissolved in 270 mM sodium phosphite buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 800-fold into 1:1 acetonitrile:water for achiral LC-MS analysis.

TABLE 11.2
Activity of Monooxygenase Variants Relative to SEQ ID NO: 410

| | Amino Acid | | |
|---|---|---|---|
| SEQ ID | Differences | FIOP (apparent % conversion to alcohol, | FIOP without heat |
| NO: | (Relative to SEQ | with thermostability challenge at 42.5° C.) | treatment Relative |
| (nt/aa) | ID NO: 410) | Relative to SEQ ID NO: 410 | to SEQ ID NO: 410 |
| 619/620 | F663G | | ++ |
| 621/622 | E860R | | ++ |
| 623/624 | E806N | | ++ |
| 625/626 | S1025G | | ++ |
| 627/628 | T960A | | ++ |
| 629/630 | N713M | | ++ |
| 631/632 | N635S | +++ | ++ |
| 633/634 | S1025W | | ++ |
| 635/636 | A783S | | ++ |
| 637/638 | K807R | | ++ |
| 639/640 | D600K | | ++ |
| 641/642 | P655T | + | ++ |
| 643/644 | D992G | | ++ |
| 645/646 | Q842G | | ++ |
| 647/648 | D894V | | ++ |
| 649/650 | A783E | | ++ |
| 651/652 | S851L | +++ | ++ |
| 653/654 | S1025T | | ++ |
| 655/656 | V1024I | | ++ |
| 657/658 | K771I | | ++ |
| 659/660 | K474P | | ++ |
| 661/662 | G677H | | ++ |
| 663/664 | T960G | | ++ |
| 665/666 | Q696C | ++++ | ++ |
| 667/668 | E840L | ++++ | |
| 669/670 | R51H/S851Q | ++++ | |
| 671/672 | E942K | ++++ | + |
| 673/674 | S664L | ++++ | + |
| 675/676 | H978S | ++++ | |
| 677/678 | V1024L | ++++ | + |
| 679/680 | P1008C | ++++ | |
| 681/682 | Q947W | ++++ | |
| 683/684 | I878S | ++++ | |
| 685/686 | K807T | ++++ | + |
| 687/688 | G677N | ++++ | + |
| 689/690 | P655A | ++++ | |
| 691/692 | N597V | ++++ | |
| 693/694 | D638W | ++++ | |
| 695/696 | N597R | ++++ | |
| 697/698 | P655L | ++++ | |
| 699/700 | T960K | ++++ | + |
| 701/702 | P460R | ++++ | |
| 703/704 | A783K | ++++ | + |
| 705/706 | D992F | +++ | + |
| 707/708 | A1012M | +++ | |
| 709/710 | K474F | +++ | |
| 711/712 | S694P | +++ | |
| 713/714 | Q466C | +++ | |
| 715/716 | A1012G | +++ | + |
| 717/718 | D638E | +++ | |
| 719/720 | G857F | +++ | |
| 721/722 | A789G | +++ | + |

Levels of increased activity were determined relative to the reference polypeptide of SEQ ID NO: 410 and defined as follows: “+” > 1 “++” > 1.15, “+++” > 1.5, “++++” > 3

### Example 12

Evolution and Screening of Engineered Polypeptides Derived from SEQ ID NO: 544 for Improved Production of Compound (2)

The engineered polynucleotide (SEQ ID NO: 543) encoding the polypeptide with monooxygenase activity of SEQ ID NO: 544 was used to generate the engineered polypeptides of Table 12.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 544, as described below.

Directed evolution began with the polynucleotide set forth in SEQ ID NO: 543. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 40 v/v % of 4-fold diluted monooxygenase lysate, prepared as described in EXAMPLE 1 and then diluted 4-fold and heated to 46° C. for 2 hours, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 500 μL/well of acetonitrile was added to the reaction plates and mixed well. The plates were sealed and centrifuged at 4,000 rpm for 10 min. An aliquot of the supernatant was removed and further diluted 800-fold into 1:1 acetonitrile:water for achiral LC-MS analysis according to the conditions in Table 2.2.

TABLE 12.1
Activity of Monooxygenase Variants Relative to SEQ ID NO: 544

| SEQ ID | | |
|---|---|---|
| NO: | Amino Acid Differences (Relative to SEQ | FIOP (% conv to alc, heat treated |
| (nt/aa) | ID NO: 544) | lysate) Relative to SEQ ID NO: 544 |
| 723/724 | E410S/A557P/E692V/P788Q | +++ |
| 725/726 | H286R/E410S/A651S/P676K | +++ |
| 727/728 | H286R/E692V/G786R/P788Q | + |
| 729/730 | E410S/A557P/P676K/P788Q/E888G/Q924N/ | +++ |
| | Q993S | |
| 731/732 | E410S/Q646F/A651S/P788Q | +++ |
| 733/734 | H286R/E410S/N707Y/P788Q | ++++ |
| 735/736 | K77L/E410S/P676K/P788Q/Q924N | ++++ |
| 737/738 | K77L/A557P/N707Y/E888G | ++ |
| 739/740 | E410S | ++ |
| 741/742 | H286R/E410S/E888G | +++ |
| 743/744 | K77L/V179L/H286R/E410S/P788Q/E888G | +++ |

Levels of increased activity were determined relative to the reference polypeptide of SEQ ID NO: 544 and defined as follows: “+” >10, “+” > 50, “+++” > 200, “++++” > 400.

### Example 13

Evolution and Screening of Engineered Polypeptides Derived from SEQ ID NO: 734 for Improved Production of Compound (2)

The engineered polynucleotide (SEQ ID NO: 733) encoding the polypeptide with monooxygenase activity of SEQ ID NO: 734 was used to generate the engineered polypeptides of Table 13.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 734, as described below.

Directed evolution began with the polynucleotide set forth in SEQ ID NO: 734. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 2 v/v % and/or 50% v/v of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 400 or 800-fold into 1:1 acetonitrile:water for achiral LC-MS analysis according to the method in Table 2.2.

Select variants were additionally analyzed for selectivity. The enzyme assays were carried out in 96-well deep-well (1.1 mL total volume) plates, in 100 μL total reaction volume per well. The reactions contained 40 v/v % of undiluted monooxygenase lysate, prepared as described in EXAMPLE 1, 10 g/L Compound (1), 1 g/L NADPH, 0.1 g/L GDH-105, 16 g/L glucose and dissolved in 100 mM potassium phosphate buffer, pH 8. The reaction plates were sealed with an aeropore seal and shaken at 600 rpm at 30° C. with 85% humidity for 18 hours.

After overnight incubation, 50 μL/well of a 25 uL/mL solution triethylamine in acetonitrile was added to the reaction plates, followed by 50 uL/well of 37 g/L solution 2-bromoacetophenone in acetonitrile. The plates were heat-sealed and shaken for 2 hours at 50° C. After 2 hours the plates were centrifuged for 10 minutes at 4,000 rpm. Then, the supernatant was filtered through a hydrophilic filter plate. An aliquot of the filtrate was removed and further diluted 50-fold into 1:1 acetonitrile:water for chiral LC-MS analysis according to the method described in Table 8.2.

TABLE 13.1
Activity of Monooxygenase Variants Relative to SEQ ID NO: 734

| | | activity FIOP, | | activity FIOP, |
|---|---|---|---|---|
| SEQ ID | | 50% lysate load | | 2% lysate load |
| NO: | Amino Acid Differences (Relative | Relative to SEQ | alcohol % | Relative to SEQ |
| (nt/aa) | to SEQ ID NO: 734) | ID NO: 734 | ee | ID NO: 734 |
| 745/746 | S88T | +++ | | +++ |
| 747/748 | S88V | ++ | | +++ |
| 749/750 | S88G | | + | |
| 751/752 | S410E/A557P/Q924G | +++ | + | |
| 753/754 | T497A/A557P | ++ | | |
| 755/756 | S410E/T497A/S814K | +++ | + | |
| 757/758 | S410E/T497A/A557P/A576V/S814K | +++ | + | |
| 759/760 | S410E/T497A/K573S/A576V | +++ | + | |
| 761/762 | T270I | | | + |
| 763/764 | S437N | | | + |
| 765/766 | S437V | | | + |
| 767/768 | T270V | | | + |
| 769/770 | D24R | | | + |
| 771/772 | Q257G | | | + |
| 773/774 | Q257N | | | + |
| 775/776 | F78E | | | + |
| 777/778 | D24S | | | + |
| 779/780 | Q257H | | | ++ |
| 781/782 | L53V | | | ++ |
| 783/784 | R180G | | | ++ |
| 785/786 | Q257W | | | ++ |
| 787/788 | F82I | | | ++ |
| 789/790 | T150G | | | ++ |
| 791/792 | D183S | | | +++ |
| 793/794 | A75G | | | +++ |

Levels of increased activity were determined relative to the reference polypeptide of SEQ ID NO: 734 and defined as follows: “+” 0.9 ++” > 1.15, “+++” > 1.5. Levels of enantiomeric excess are measured as % ee and defined as follows: “ “+” > 80%. SEQ ID NO: 734 has 80% ee.

### Example 14

Evolution and Screening of Engineered Polypeptides Derived from SEQ ID NO: 748 for Improved Production of Compound (2)

The engineered polynucleotide (SEQ ID NO: 747) encoding the polypeptide with monooxygenase activity of SEQ ID NO: 748 was used to generate the engineered polypeptides of Tables 14.1 and 14.2. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 748, as described below.

Directed evolution began with the polynucleotide set forth in SEQ ID NO: 748. Libraries of engineered polypeptides were generated using various well-known techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. These variants, and all variants in further examples, were screened using phosphite dehydrogenase (PDH) and phosphite to regenerate the NADPH cofactor instead of glucose dehydrogenase (GDH) and glucose. The switch was made due to superior performance of the SFPs with under PDH conditions. The reactions contained 1 v/v % of undiluted monooxygenase lysate, prepared as described in EXAMPLE 1, 10 g/L Compound (1), 1 g/L NADPH, 0.25 g/L PDH-102, and dissolved in 500 mM sodium phosphite buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 800-fold into 1:1 acetonitrile:water for achiral LC-MS analysis according to the conditions in Table 2.2.

TABLE 14.1
Activity of Monooxygenase Variants Relative to SEQ ID NO: 748

| | | FIOP, % |
|---|---|---|
| | | conversion to |
| SEQ ID | | alcohol, 1% |
| NO: | | lysate Relative to |
| (nt/aa) | Amino Acid Differences (Relative to SEQ ID NO: 748) | SEQ ID NO: 748 |
| 795/796 | A75G/Q257N/T613A | + |
| 797/798 | R180G/S437N/V773A/L795A | +++ |
| 799/800 | Q257H/T613A/V773A/L795A | + |
| 801/802 | A75G/F82I/Q257G/Q268T | + |
| 803/804 | A75G/Q257N/K640W | ++ |
| 805/806 | A75G/R180G/D183S/Q257N/Q268T/T270I/A385R/S437N/S556G/T613A/ | + |
| | A652S/E923A | |
| 807/808 | A75G/F82I/R180G/Q257G/S437N | + |
| 809/810 | A75G | + |
| 811/812 | A75G/F82I/Q257N/S556G/K640G | ++ |
| 813/814 | A75G/S556G/V773A | + |
| 815/816 | A75G/R180G/Q257N/Q268T/T270I/S437V/S556G/N574S/A652S | + |
| 817/818 | A75G/F82I/S437N | ++ |
| 819/820 | A75G/N574S | ++ |
| 821/822 | F82I/T613A | + |
| 823/824 | A75G/F82I/Q257H/V773A | + |
| 825/826 | A75G/R180G/N574S/L795A | ++ |
| 827/828 | A75G/T613A | +++ |

Levels of increased activity were determined relative to the reference polypeptide of SEQ ID NO: 748 and defined as follows, “+” > 1.15, “+” > 3, “+” > 5.

Other variants derived from SEQ ID NO: 747 were carried out in 96-well deep-well (1.1 mL total volume) plates, in 100 μL total reaction volume per well. The reactions contained 10 v/v % of undiluted monooxygenase lysate, prepared as described in EXAMPLE 1, 25 g/L Compound (1), 1 g/L NADPH, 0.5 g/L PDH-102, and dissolved in 450 mM sodium phosphite buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 100-fold into 1:1 acetonitrile:water for achiral LC-MS analysis according to the conditions in Table 2.2.

TABLE 14.2
Activity of Monooxygenase Variants Relative to SEQ ID NO: 748

| SEQ ID | | |
|---|---|---|
| NO: | Amino Acid Differences (Relative | FIOP, % conversion to alcohol, 10% lysate |
| (nt/aa) | to SEQ ID NO: 748) | Relative to SEQ ID NO: 748 |
| 829/830 | Q237N | ++ |
| 831/832 | P244G | + |
| 833/834 | G114M | + |
| 835/836 | Q110V | +++ |
| 837/838 | E248M | + |
| 839/840 | N102A | ++ |
| 841/842 | P347E | + |
| 843/844 | P347S | + |
| 845/846 | C206R | ++ |
| 847/848 | P106V | + |
| 849/850 | N102L | + |
| 851/852 | D196Q/P853L | + |
| 853/854 | E245A | + |
| 855/856 | E245M | + |
| 857/858 | P244R | ++ |
| 859/860 | E245S | ++ |
| 861/862 | L127M | + |
| 863/864 | P194T | + |
| 865/866 | R191S | + |
| 867/868 | T236G | + |
| 869/870 | E245G | + |
| 871/872 | G114R | +++ |
| 873/874 | H232R | + |
| 875/876 | I210T | ++ |
| 877/878 | E245N | ++ |
| 879/880 | N193K | + |
| 881/882 | A45E | + |
| 883/884 | K203G | + |
| 885/886 | Q110L | +++ |
| 887/888 | E245R | ++ |
| 889/890 | E245T | ++ |
| 891/892 | Q237R | + |
| 893/894 | E245Q | ++ |
| 895/896 | E248R | + |
| 897/898 | E245W | ++ |
| 899/900 | R256K | + |
| 901/902 | G114H | + |
| 903/904 | Q110F | ++ |
| 905/906 | P347K | + |
| 907/908 | A226N | + |
| 909/910 | N254L | + |
| 911/912 | T236S | + |
| 913/914 | P244Y | ++ |
| 915/916 | I210R | + |
| 917/918 | E245L | ++ |
| 919/920 | H111R | + |
| 921/922 | H111L | ++ |
| 923/924 | E245V | ++ |
| 925/926 | G114Q | + |
| 927/928 | H111W | ++ |
| 929/930 | D196W | + |
| 931/932 | A198S | + |
| 933/934 | N202Q | + |

Levels of increased activity were determined relative to the reference polypeptide of SEQ ID NO: 748 and defined as follows: “+” > 1.2, “++” > 1.5, “+++” > 2.

### Example 15

Evolution and Screening of Engineered Polypeptides Derived from SEQ ID NO: 828 for Improved Production of Compound (2)

The engineered polynucleotide (SEQ ID NO: 827) encoding the polypeptide with monooxygenase activity of SEQ ID NO: 828 was used to generate the engineered polypeptides of Table 15.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 828, as described below.

Directed evolution began with the polynucleotide set forth in SEQ ID NO: 827. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 5 v/v % of undiluted monooxygenase lysate, prepared as described in EXAMPLE 1, 25 g/L Compound (1), 1 g/L NADPH, 0.5 g/L PDH-102, and dissolved in 475 mM sodium phosphite buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 800-fold into 1:1 acetonitrile:water for achiral LC-MS analysis according to the conditions in Table 2.2.

TABLE 15.1
Activity of Monooxygenase Variants Relative to SEQ ID NO: 828

| SEQ ID | Amino Acid Differences (Relative to SEQ | FIOP, % conversion to alcohol Relative |
|---|---|---|
| NO: (nt/aa) | ID NO: 828) | to SEQ ID NO: 828 |
| 935/936 | R826G/S1004I | + |
| 937/938 | V779F/S1004I | ++ |
| 939/940 | D550T/E616L/A717I/V779F | ++ |
| 941/942 | N22D/G75A/A717C/G720L/V779F/S1004I | + |
| 943/944 | D550T/A717C/L795V/L799I/T800Y | + |
| 945/946 | E616L/A717G/G720L/L799I | + |
| 947/948 | N22D/D550T/R826G | + |
| 949/950 | A717G/S1004I | +++ |
| 951/952 | A717C | + |
| 953/954 | A717G/G720L/V779F/S1004I | ++ |
| 955/956 | A717Q/V779F/L799I/T800Y/S1004I | +++ |
| 957/958 | G720L/V779F | + |
| 959/960 | N22D/A717Q/L795V/L799I/R826G | + |
| 961/962 | N22D/E616L/A717I/S1004I | |
| 963/964 | T800Y/S1004I | ++ |
| 965/966 | K640V | ++ |
| 967/968 | A717Q/S1004I | +++ |
| 969/970 | D550T/K640D/A717I | +++ |

Levels of increased activity were determined relative to the reference polypeptide of SEQ ID NO: 828 and defined as follows: “+” > 1.2, “++” > 1.5, +++++ > 1.6.

### Example 16

Evolution and Screening of Engineered Polypeptides Derived from SEQ ID NO: 968 for Improved Production of Compound (2)

The engineered polynucleotide (SEQ ID NO: 967) encoding the polypeptide with monooxygenase activity of SEQ ID NO: 968 was used to generate the engineered polypeptides of Table 16.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 968, as described below.

Directed evolution began with the polynucleotide set forth in SEQ ID NO: 967. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 5 v/v % of undiluted monooxygenase lysate, prepared as described in EXAMPLE 1, 25 g/L Compound (1), 1 g/L NADPH, 0.5 g/L PDH-102, and dissolved in 475 mM sodium phosphite buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 800-fold into 1:1 acetonitrile:water for achiral LC-MS analysis according to the conditions in Table 2.2.

TABLE 16.1
Activity of Monooxygenase Variants Relative to SEQ ID NO: 968

| SEQ ID | Amino Acid Differences (Relative to | FIOP, % conversion to alcohol Relative |
|---|---|---|
| NO: (nt/aa) | SEQ ID NO: 968) | to SEQ ID NO: 968 |
| 971/972 | G786A/E840S/T960L | ++ |
| 973/974 | T960L | ++ |
| 975/976 | D638G/F663R/Q793T/E840S | ++ |
| 977/978 | N635S/S636G/Q793T/E840L | ++ |
| 979/980 | E840L/T960L | ++ |
| 981/982 | N635S/D638G/Q793T/P823A/T960L | ++ |
| 983/984 | D600K/E840L/T960L | ++ |
| 985/986 | Q793T/E840L | ++ |
| 987/988 | S636M/D638G/Q793T/S851L | ++ |
| 989/990 | S851L/V1024I/S1025T | ++ |
| 991/992 | S1025W | ++ |
| 993/994 | K807R | ++ |
| 995/996 | N713M | ++ |
| 997/998 | S851L/S1025T | ++ |
| 999/1000 | K771I | ++ |
| 1001/1002 | N635S/N713M/S1025T | ++ |
| 1003/1004 | D600K/N635S/N713M/K771I/S1025G | ++ |
| 1005/1006 | S1025T | ++ |
| 1007/1008 | N635S/K771I/D894V | ++ |
| 1009/1010 | V446S | + |
| 1011/1012 | S305G | ++ |
| 1013/1014 | S305R | ++ |
| 1015/1016 | K307R | ++ |
| 1017/1018 | V300P | ++ |
| 1019/1020 | A468F | ++ |
| 1021/1022 | T366S | ++ |
| 1023/1024 | A118T/V446L | +++ |
| 1025/1026 | N382M | ++ |
| 1027/1028 | E132V | ++ |
| 1029/1030 | I3W | ++ |
| 1031/1032 | K473L/Y790H | +++ |
| 1033/1034 | P285G | ++ |
| 1035/1036 | E381F | ++ |
| 1037/1038 | L415V | ++ |
| 1039/1040 | F424Y | ++ |
| 1041/1042 | E132M | ++ |
| 1043/1044 | T366I | +++ |
| 1045/1046 | V303W | ++ |
| 1047/1048 | A295S | + |
| 1049/1050 | Q230D | + |
| 1051/1052 | V433D | ++ |
| 1053/1054 | M417V | ++ |
| 1055/1056 | E293T | ++ |
| 1057/1058 | Q230V | + |
| 1059/1060 | V303Q | ++ |
| 1061/1062 | K290S | ++ |
| 1063/1064 | A296G | ++ |
| 1065/1066 | P285S | ++ |
| 1067/1068 | D371K | ++ |
| 1069/1070 | T481L | ++ |
| 1071/1072 | A292S | + |
| 1073/1074 | A296Q | + |
| 1075/1076 | S463V | + |
| 1077/1078 | A478S | ++ |
| 1079/1080 | S463A | +++ |
| 1081/1082 | P455R | + |
| 1083/1084 | M506V | ++ |
| 1085/1086 | F424V | ++ |
| 1087/1088 | V433K | +++ |
| 1089/1090 | E293R | ++ |
| 1091/1092 | M418V | ++ |
| 1093/1094 | N382T | ++ |
| 1095/1096 | E465Q | ++ |
| 1097/1098 | V372S | ++ |
| 1099/1100 | V433L | ++ |
| 1101/1102 | M506D | ++ |
| 1103/1104 | I3D | ++ |
| 1105/1106 | V447L | ++ |
| 1107/1108 | L432V | ++ |
| 1109/1110 | N480W | ++ |
| 1111/1112 | M418L | ++ |
| 1113/1114 | H427T | ++ |
| 1115/1116 | S305N | ++ |
| 1117/1118 | K473R | ++ |
| 1119/1120 | V433R | ++ |

Levels of increased activity were determined relative to the reference polypeptide of SEQ ID NO: 968 and defined as follows: “+” > 1,”++” > 1.15, “+++” > 1.5.

### Example 17

Evolution and Screening of Engineered Polypeptides Derived from SEQ ID NO:984 for Improved Production of Compound (2)

The engineered polynucleotide (SEQ ID NO: 983) encoding the polypeptide with monooxygenase activity of SEQ ID NO: 984 was used to generate the engineered polypeptides of Tables 17.1 and 17.2. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 984 as described below.

Directed evolution began with the polynucleotide set forth in SEQ ID NO: 983. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in 96-well deep-well (1.1 mL total volume) plates, in 100 μL total reaction volume per well. The reactions contained 5 (Table 17.1) or 10 v/v % (Table 17.2) of undiluted monooxygenase lysate, prepared as described in EXAMPLE 1, 25 g/L Compound (1), 1 g/L NADPH, 0.5 g/L PDH-102, and dissolved in 450 mM sodium phosphite buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 800-fold into 1:1 acetonitrile:water for achiral LC-MS analysis according to the conditions in Table 2.2.

TABLE 17.1
Activity of Monooxygenase Variants Relative to SEQ ID NO: 984

| SEQ ID | | |
|---|---|---|
| NO: | Amino Acid Differences (Relative | FIOP, % conversion to alcohol (5% lysate) |
| (nt/aa) | to SEQ ID NO: 984) | Relative to SEQ ID NO: 984 |
| 1121/1122 | Q110L/G114Q | + |
| 1123/1124 | E245V | ++ |
| 1125/1126 | Q237R | ++ |
| 1127/1128 | G114Q | + |
| 1129/1130 | E245S | ++ |
| 1131/1132 | Q237R/E245V | ++ |
| 1133/1134 | G114Q/E245V | ++ |
| 1135/1136 | A45E/P853L | ++ |
| 1137/1138 | H111R/I210T/P347S/P853L | ++ |
| 1139/1140 | H111L | ++ |
| 1141/1142 | H111L/L127M/A226N/P244Y | ++ |
| 1143/1144 | I210T/P853L | + |
| 1145/1146 | H111W/A226N/P853L/P969S | ++ |
| 1147/1148 | L127M/P347E | ++ |
| 1149/1150 | H111R/P853L | +++ |
| 1151/1152 | L127M/I210T/P244Y/P853L | +++ |
| 1153/1154 | A45E | ++ |
| 1155/1156 | L127M/I210T/P244Y | ++ |
| 1157/1158 | H111R/A226N/P244Y/P347S | ++ |
| 1159/1160 | H111R/A226N | ++ |
| 1161/1162 | H111R/P347E/P853L | ++ |
| 1163/1164 | P194T/P853L | ++ |
| 1165/1166 | H111L/I210T | + |
| 1167/1168 | H111R/P194T/P347S | + |
| 1169/1170 | H111R/L127M/A226N/P244Y | ++ |
| 1171/1172 | A226N/T236G/P244Y/P347S/P853L | ++ |
| 1173/1174 | P244Y/P853L | ++ |
| 1175/1176 | H111W | ++ |
| 1177/1178 | H111R/P194T/P244Y/P347S/P853L | ++ |
| 1179/1180 | H111L/P244R | + |
| 1181/1182 | T236G/P244Y | +++ |
| 1183/1184 | H111L/P853L | ++++ |
| 1185/1186 | H111R/P194T/P347S/P853L | + |
| 1187/1188 | L127M/I210T/P347E/P853L | +++ |
| 1189/1190 | L127M/P244Y | ++ |
| 1191/1192 | H111L/P244Y/P853L | + |
| 1193/1194 | A45E/H111R/A226N/P347S | + |

Levels of increased activity were determined relative to the reference polypeptide of SEQ ID NO: 984 and defined as follows: “+” > 1, “++” > 1.15, “+++” > 1.5, “++++” > 2.

TABLE 17.2
Activity of Monooxygenase Variants Relative to SEQ ID NO: 984

| SEQ ID | | |
|---|---|---|
| NO: | Amino Acid Differences (Relative | FIOP, % conversion to alcohol (10% lysate) |
| (nt/aa) | to SEQ ID NO: 984) | Relative to SEQ ID NO: 984 |
| 1195/1196 | D653T | + |
| 1197/1198 | M621T | + |
| 1199/1200 | G562P | + |
| 1201/1202 | V563S | + |
| 1203/1204 | N666R | + |
| 1205/1206 | K640L | + |
| 1207/1208 | D653A | + |
| 1209/1210 | D619G | + |
| 1211/1212 | D619R | + |
| 1213/1214 | D653R | + |
| 1215/1216 | S623P | + |
| 1217/1218 | Y628S | + |
| 1219/1220 | S519L | + |
| 1221/1222 | R618L | + |
| 1223/1224 | R618G | + |
| 1225/1226 | D518T | + |
| 1227/1228 | D518N/A652S | + |
| 1229/1230 | D518S | + |
| 1231/1232 | D518G | + |
| 1233/1234 | A584G/S724P | + |
| 1235/1236 | V563L | + |
| 1237/1238 | E616V | ++ |
| 1239/1240 | I586V | ++ |

Levels of increased activity were determined relative to the reference polypeptide of SEQ ID NO: 984 and defined as follows: “+” > 1.15, “++” > 1.5.

### Example 18

Evolution and Screening of Engineered Polypeptides Derived from SEQ ID NO: 1160 for Improved Production of Compound (2)

The engineered polynucleotide (SEQ ID NO: 1159) encoding the polypeptide with monooxygenase activity of SEQ ID NO: 1160 was used to generate the engineered polypeptides of Table 18.1. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 1160, as described below.

Directed evolution began with the polynucleotide set forth in SEQ ID NO: 1159. Libraries of engineered polypeptides were generated using various techniques (e.g., saturation mutagenesis, recombination of previously identified beneficial amino acid differences) and screened using HTP assay and analysis methods that measured the polypeptides' ability to produce Compound (2).

The enzyme assays were carried out in duplicate in 96-well deep-well (1.1 mL total volume) plates, in 100 μL total reaction volume per well. The reactions contained 5 v/v % of undiluted monooxygenase lysate, prepared as described in EXAMPLE 1, 25 g/L Compound (1), 1 g/L NADPH, 0.5 g/L PDH-102, and dissolved in 475 mM sodium phosphite buffer. One set of reactions was set up at pH 7.5 and the other duplicate set of reactions was set up at pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 800-fold into 1:1 acetonitrile:water for achiral LC-MS analysis according to the conditions in Table 2.2.

TABLE 18.1
Activity of Monooxygenase Variants Relative to SEQ ID NO: 1160

| | | FIOP, pH 8.0 | FIOP, pH 7.5 |
|---|---|---|---|
| SEQ ID | Amino Acid Differences (Relative to | Relative to SEQ ID | Relative to SEQ ID |
| NO: (nt/aa) | SEQ ID NO: 1160) | NO: 1160 | NO: 1160 |
| 1241/1242 | S1025T | +++ | + |
| 1243/1244 | E132G/A468F/Q793T | ++ | |
| 1245/1246 | V433K/S467K/S1025T | +++ | + |
| 1247/1248 | E132G/T366I/V433K/S463A/S467K/Q793T | ++ | ++ |
| 1249/1250 | K290T/S1025T | ++ | ++ |
| 1251/1252 | K290T/T366I/V433K/S463A/S467K | ++ | |
| 1253/1254 | V433K | +++ | + |
| 1255/1256 | K290T/V433K/S467K/Q793T/S1025T | | ++ |
| 1257/1258 | E132G/S1025T | +++ | ++ |
| 1259/1260 | D183G/S1025A/D1045N/L1048R | + | +++ |
| 1261/1262 | V433K/Y790H | | +++ |
| 1263/1264 | K290T/V433K/Q793T | + | ++ |
| 1265/1266 | E132G/T366I/S467G/G661D | + | +++ |
| 1267/1268 | Q793T | + | + |
| 1269/1270 | E132G/S467K/A468F/M506V/Q793T | +++ | ++ |
| 1271/1272 | K473L | | +++ |
| 1273/1274 | S463A/Q793T | + | ++ |
| 1275/1276 | V433K/M506V/S1025T | + | +++ |
| 1277/1278 | K290T/Q793T | ++ | +++ |
| 1279/1280 | T366I/V433K | ++ | + |

Levels of increased activity were determined relative to the reference polypeptide of SEQ ID NO: 1160 and defined as follows: “+” > 1, “++” > 1.15, “+++” > 1.3.

### Example 19

Evolution and Screening of Engineered Polypeptides Derived from SEQ ID NO: 1266 for Improved Production of Compound (2)

The engineered polynucleotide (SEQ ID NO: 1265) encoding the polypeptide with monooxygenase activity of SEQ ID NO: 1266 was used to generate the engineered polypeptides of Tables 19.1 and 19.2. These polypeptides displayed improved monooxygenase activity under the desired conditions e.g., the improvement in the formation of the alcohol Compound (2), from the substrate Compound (1), as compared to the starting polypeptide. The engineered polypeptides, having the amino acid sequences of even-numbered sequence identifiers were generated from the “backbone” amino acid sequence of SEQ ID NO: 1266, as described below.

Directed evolution began with the polynucleotide set forth in SEQ ID NO: 1265. Libraries of engineered polypeptides were generated using various techniques (e.g., saturation mutagenesis, recombination of previously identified beneficial amino acid differences) and screened using HTP assay and analysis methods that measured the polypeptides, ability to produce Compound (2).

The enzyme assays were carried out in 96-well deep-well (1.1 mL total volume) plates, in 100 μL total reaction volume per well. The reactions contained 10 v/v % of undiluted monooxygenase lysate, prepared as described in EXAMPLE 1, 25 g/L Compound (1), 1 g/L NADP+, 0.5 g/L PDH-102, and dissolved in 450 mM sodium phosphite buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 800-fold into 1:1 acetonitrile:water for achiral LC-MS analysis according to the conditions in Table 2.2.

TABLE 19.1
Activity of Monooxygenase Variants Relative to SEQ ID NO: 1266

| SEQ ID | | pos control fiop % conv |
|---|---|---|
| NO: | Amino Acid Differences (Relative to SEQ ID NO: | alcohol Relative to SEQ ID |
| (nt/aa) | 1266) | NO: 1266 |
| 1253/1254 | G132E/I366T/V433K/G467S/D661G | + |
| 1247/1248 | V433K/S463A/G467K/D661G/Q793T | + |
| 1249/1250 | G132E/K290T/I366T/G467S/D661G/S1025T | + |
| 1255/1256 | G132E/K290T/I366T/V433K/G467K/D661G/Q793T/S1025T | + |
| 1259/1260 | G132E/D183G/366T/G467S/D661G/S1025A/D1045N/L1048R | + |
| 1261/1262 | G132E/I366T/V433K/G467S/D661G/Y790H | + |
| 1265/1266 | | + |
| 1267/1268 | G132E/I366T/G467S/D661G/Q793T | + |
| 1269/1270 | I366T/G467K/A468F/M506V/D661G/Q793T | + |
| 1271/1272 | G132E/I366T/G467S/K473L/D661G | + |
| 1273/1274 | G132E/I366T/S463A/G467S/D661G/Q793T | + |
| 1275/1276 | G132E/I366T/V433K/G467S/M506V/D661G/S1025T | + |
| 1277/1278 | G132E/K290T/I366T/G467S/D661G/Q793T | + |
| 1281/1282 | S463V/P853L | |
| 1283/1284 | S463V | + |
| 1285/1286 | G114R/Q230V/V446S/P853L | + |
| 1287/1288 | G114R/A292S/E293T/A296Q/S463V/P853L | ++ |
| 1289/1290 | P853L | + |
| 1291/1292 | S1017G | + |
| 1293/1294 | H1022W | +++ |
| 1295/1296 | S1017L | ++ |
| 1297/1298 | E792V | ++ |
| 1299/1300 | A1027R | + |
| 1301/1302 | H1022S | + |
| 1303/1304 | L1031Q | +++ |
| 1305/1306 | E981N | + |
| 1307/1308 | Q926R | ++ |
| 1309/1310 | A769T | +++ |
| 1311/1312 | S939D | ++ |
| 1313/1314 | E923V | + |
| 1315/1316 | E792I | ++ |
| 1317/1318 | M1015L | ++ |
| 1319/1320 | S824G | ++ |
| 1321/1322 | T974V | + |
| 1323/1324 | S1017A | ++ |
| 1325/1326 | M968L | ++ |
| 1327/1328 | H962R | ++ |
| 1329/1330 | N952G | + |
| 1331/1332 | S724W | +++ |
| 1333/1334 | A810S | + |
| 1335/1336 | G1040R | ++ |
| 1337/1338 | E780I | ++ |
| 1339/1340 | E730W | ++ |
| 1341/1342 | G720Q | +++ |
| 1343/1344 | E817G | ++ |
| 1345/1346 | A995G | ++ |
| 1347/1348 | E923R | ++ |
| 1349/1350 | L689G | + |
| 1351/1352 | G1040D | +++ |
| 1353/1354 | V979Q | +++ |
| 1355/1356 | M1006S | ++++ |

Levels of increased activity were determined relative to the reference polypeptide of SEQ ID NO: 1266 and defined as follows: “+” > 1.2, “++” > 1.5, “+++” > 2, “++++” > 3.

Other variants derived from SEQ ID NO: 1265 were analyzed. The enzyme assays were carried out in 96-well deep-well (1.1 mL total volume) plates, in 100 μL total reaction volume per well. The reactions contained 40 v/v % of undiluted monooxygenase lysate, prepared as described in EXAMPLE 1, 25 g/L Compound (1), 1 g/L NADPH, 0.5 g/L PDH-102, and dissolved in 300 mM sodium phosphite buffer, pH 8. The reaction plates were sealed with an aeropore seal and shaken at 600 rpm at 30° C. with 85% humidity for 18 hours.

After overnight incubation, 400 μL/well of acetonitrile was added to the reaction plates and mixed well. The plates were sealed and centrifuged at 4,000 rpm for 10 min. An aliquot of the supernatant was removed and further diluted 400-fold into 1:1 acetonitrile:water for achiral LC-MS analysis according to the conditions in Table 2.2.

TABLE 19.2
Activity of Monooxygenase Variants Relative to SEQ ID NO: 1266

| SEQ ID | Amino Acid Differences (Relative to | FIOP, % conversion to alcohol Relative |
|---|---|---|
| NO: (nt/aa) | SEQ ID NO: 1266) | to SEQ ID NO: 1266 |
| 1357/1358 | D518G/D653A | + |
| 1359/1360 | D518G/D653R | + |
| 1361/1362 | D653R | + |
| 1363/1364 | E616V/D619G | ++ |
| 1365/1366 | G458A | + |
| 1367/1368 | S519L/Y628S | + |

Levels of increased activity were determined relative to the reference polypeptide of SEQ ID NO: 1266 and defined as follows: “+” >1.2,”++” > 1.5.

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes in the United States of America, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

What is claimed is:

1. An engineered cytochrome P450-BM3 variant comprising a polypeptide sequence having at least 85%, 86%, 87%, 88%, 89%, 90%, 91%, 92%, 93%, 94%, 95%, 96%, 97%, 98%, 99%, or more sequence identity to SEQ ID NO: 36, 4, 66, 72, 198, 226, 244, 286, 358, 410, 534, 734, 748, 828, 968, 984, 1160, or 1266, or a functional fragment thereof, wherein the polypeptide sequence of said engineered cytochrome P450-BM3 variant comprises at least one substitution or substitution set and wherein the amino acid positions of said polypeptide sequence are numbered with reference to SEQ ID NO: 36, 4, 66, 72, 198, 226, 244, 286, 358, 410, 534, 734, 748, 828, 968, 984, 1160, or 1266.

2. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 4, and wherein the polypeptide sequence of said engineered cytochrome P450-BM3 variant comprises at least one substitution or substitution set at one or more positions in said polypeptide sequence selected from 52/83/88/105, 32/83/88, 32/83/88/176, 32/83/88/231/574, 32/83/88/574, 52/83/88, 52/83/88/231, 52/83/88/231/433/574, 52/83/88/433, 52/83/88/433/574, 52/83/88/574, 83/88, 83/88/105, 83/88/111, 83/88/111/433, 83/88/111/574, 83/88/231, 83/88/349, 83/88/433/574, and 83/88/574, wherein the amino acid positions of said polypeptide sequence are numbered with reference to SEQ ID NO: 4.

3. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 36, and wherein the polypeptide sequence of said engineered cytochrome P450-BM3 variant comprises at least one substitution or substitution set at one or more positions in said polypeptide sequence selected from 75, 75/374, 75/374/458/726, 75/374/726, 75/458, 75/458/726, 75/726, 111/114, 111/603/604/623/853, 111/623, 374/726, and 726, wherein the amino acid positions of said polypeptide sequence are numbered with reference to SEQ ID NO: 36.

4. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 66, and wherein the polypeptide sequence of said engineered cytochrome P450-BM3 variant comprises at least one substitution or substitution set at one or more positions in said polypeptide sequence selected from 74, 75, 83, 179, 181, 182, 186, 189, 238, 267, 268, 328, 331, 355, 358, 437, and 438, wherein the amino acid positions of said polypeptide sequence are numbered with reference to SEQ ID NO: 66.

5. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 72, and wherein the polypeptide sequence of said engineered cytochrome P450-BM3 variant comprises at least one substitution or substitution set at one or more positions in said polypeptide sequence selected from 74/75/83/179/189/328/331/437, 74/75/268/328/331/358/437, 74/83/268/328/331/358/437, 74/267/268/328, 75/83/179/189/331, 75/83/179/268/328/331/437, 75/83/189/267/268, 75/83/267/268/355/358/654, 75/83/268/437, 75/268/328/331/358, 83/179/182/437, 83/179/189/331/355, 83/179/328/331, 83/179/355/358/437, 83/182/189/268/328/331/355/358/437, 83/182/189/328/331, 83/182/268/328/355/358/437, 83/189/267/268/358, 83/189/268/328/331, 83/189/268/328/355/358/437, 83/189/328/331/437, 83/267/268/328/331/355/358, 83/268, 83/268/328/331, 83/268/328/331/355/358, 83/268/328/331/358, 83/268/328/331/358/437, 83/268/328/331/437, 83/268/331, 83/331, 83/331/437, 83/358/437, 179/182/268, 179/189/331/437, 179/328/331, 179/331/358, 189/267/268/437, 189/268/328, 189/268/328/331/358/437, 189/268/358, 267/268/328, 267/268/328/331/355/358, 267/268/331/355/358/437, 268, 268/328/331, 268/328/355/358, 268/331/355/358, 268/355/358/437, 268/358, and 328/331/358, wherein the amino acid positions of said polypeptide sequence are numbered with reference to SEQ ID NO: 72.

6. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 198, and wherein the polypeptide sequence of said engineered cytochrome P450-BM3 variant comprises at least one substitution or substitution set at one or more positions in said polypeptide sequence selected from 79, 213, and 257, wherein the amino acid positions of said polypeptide sequence are numbered with reference to SEQ ID NO: 198.

7. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 226, and wherein the polypeptide sequence of said engineered cytochrome P450-BM3 variant comprises at least one substitution or substitution set at one or more positions in said polypeptide sequence selected from 315, 320, 385, 388, 391, 398, 405, 493, 497, 502, 503, 504, 541, 542, 547, 573, 576, and 577, wherein the amino acid positions of said polypeptide sequence are numbered with reference to SEQ ID NO: 226.

8. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 244, and wherein the polypeptide sequence of said engineered cytochrome P450-BM3 variant comprises at least one substitution or substitution set at one or more positions in said polypeptide sequence selected from 75/178/213/315, 75/331/576/726, 178, 178/179/213/437/497/573/576, 178/179/573/726, 178/179/576, 178/213/573, 178/213/726, 178/437, 178/497/726, 178/576, 178/726, 179/358, 179/726, 331, 331/358/391/437, 331/497, 331/573/576, 497/573, 573, 682, 685, 699, 701, 704, 707, 708, 726, 756, 759, 794, 796, 797, 848, 851, 862, 888, 889, 999, 1003, and 1048, wherein the amino acid positions of said polypeptide sequence are numbered with reference to SEQ ID NO: 244.

9. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 286, and wherein the polypeptide sequence of said engineered cytochrome P450-BM3 variant comprises at least one substitution or substitution set at one or more positions in said polypeptide sequence selected from 75/331/701/726/851/1048, 75/331/726/999, 75/726/796/851/999, 75/1048, 87, 88/522, 89, 234, 269, 328, 330, 331, 398, 405, 408, and 411, wherein the amino acid positions of said polypeptide sequence are numbered with reference to SEQ ID NO: 286.

10. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 358, and wherein the polypeptide sequence of said engineered cytochrome P450-BM3 variant comprises at least one substitution or substitution set at one or more positions in said polypeptide sequence selected from 77, 170, 286, 289, 462, 547, 557, 630, 646, 651, 672, 676, 692, 775, 786, 787, 788, 814, 841, 876, 877, 888, 893, 896, 924, 941, 955, 969, 973, 982, 989, 993, 1038, 75/269/707, 269, 269/522/707, 269/522/707/1048, 522, 522/726, 522/1048, and 726/1048, wherein the amino acid positions of said polypeptide sequence are numbered with reference to SEQ ID NO: 358.

11. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 410, and wherein the polypeptide sequence of said engineered cytochrome P450-BM3 variant comprises at least one substitution or substitution set at one or more positions in said polypeptide sequence selected from 158/170, 158/170/410/462/630/672/726/786/788/814/924, 158/170/410/462/726/786/814, 158/170/410/924, 158/170/630/726/786/788/814, 158/410, 158/410/462/557/969, 158/410/557/786/787, 158/410/814/924, 158/410/862, 158/462/557/630/924, 158/462/630/786/788/969, 158/557, 158/557/630/814, 158/557/726/786/788/862, 158/557/786, 158/557/786/787/788, 158/557/814/924, 158/630/786/814, 158/726, 158/786/787/788, 158/814/924, 170/410/557/786/787/814/924/989, 385, 410/462/557/630/786/787/969, 462/557/726/786/787/924, 469, 523, 550, 553, 556, 574, 613, 640, 645, 650, 652, 717, 773, 779, 795, 838, 871, 923, 926, 51/851, 460, 466, 474, 597, 600, 635, 638, 655, 663, 664, 677, 694, 696, 713, 771, 783, 789, 806, 807, 840, 842, 851, 857, 860, 878, 894, 942, 947, 960, 978, 992, 1008, 1012, 1024, and 1025, wherein the amino acid positions of said polypeptide sequence are numbered with reference to SEQ ID NO: 410.

12. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 544, and wherein the polypeptide sequence of said engineered cytochrome P450-BM3 variant comprises at least one substitution or substitution set at one or more positions in said polypeptide sequence selected from 77/179/286/410/788/888, 77/410/676/788/924, 77/557/707/888, 286/410/651/676, 286/410/707/788, 286/410/888, 286/692/786/788, 410, 410/557/676/788/888/924/993, 410/557/692/788, and 410/646/651/788, wherein the amino acid positions of said polypeptide sequence are numbered with reference to SEQ ID NO: 544.

13. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 734, and wherein the polypeptide sequence of said engineered cytochrome P450-BM3 variant comprises at least one substitution or substitution set at one or more positions in said polypeptide sequence selected from 24, 53, 75, 78, 82, 88, 150, 180, 183, 257, 270, 410/497/557/576/814, 410/497/573/576, 410/497/814, 410/557/924, 437, and 497/557, wherein the amino acid positions of said polypeptide sequence are numbered with reference to SEQ ID NO: 734.

14. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 748, and wherein the polypeptide sequence of said engineered cytochrome P450-BM3 variant comprises at least one substitution or substitution set at one or more positions in said polypeptide sequence selected from 75, 75/82/180/257/437, 75/82/257/268, 75/82/257/556/640, 75/82/257/773, 75/82/437, 75/180/183/257/268/270/385/437/556/613/652/923, 75/180/257/268/270/437/556/574/652, 75/180/574/795, 75/257/613, 75/257/640, 75/556/773, 75/574, 75/613, 82/613, 180/437/773/795, 257/613/773/795, 45, 102, 106, 110, 111, 114, 127, 191, 193, 194, 196, 196/853, 198, 202, 203, 206, 210, 226, 232, 236, 237, 244, 245, 248, 254, 256, and 347, wherein the amino acid positions of said polypeptide sequence are numbered with reference to SEQ ID NO: 748.

15. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 828, and wherein the polypeptide sequence of said engineered cytochrome P450-BM3 variant comprises at least one substitution or substitution set at one or more positions in said polypeptide sequence selected from 22/75/717/720/779/1004, 22/550/826, 22/616/717/1004, 22/717/795/799/826, 550/616/717/779, 550/640/717, 550/717/795/799/800, 616/717/720/799, 640, 717, 717/720/779/1004, 717/779/799/800/1004, 717/1004, 720/779, 779/1004, 800/1004, and 826/1004, wherein the amino acid positions of said polypeptide sequence are numbered with reference to SEQ ID NO: 828.

16. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 968, and wherein the polypeptide sequence of said engineered cytochrome P450-BM3 variant comprises at least one substitution or substitution set at one or more positions in said polypeptide sequence selected from 3, 118/446, 132, 230, 285, 290, 292, 293, 295, 296, 300, 303, 305, 307, 366, 371, 372, 381, 382, 415, 417, 418, 424, 427, 432, 433, 446, 447, 455, 463, 465, 468, 473, 473/790, 478, 480, 481, 506, 600/635/713/771/1025, 600/840/960, 635/636/793/840, 635/638/793/823/960, 635/713/1025, 635/771/894, 636/638/793/851, 638/663/793/840, 713, 771, 786/840/960, 793/840, 807, 840/960, 851/1024/1025, 851/1025, 960, and 1025, wherein the amino acid positions of said polypeptide sequence are numbered with reference to SEQ ID NO: 968.

17. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 984, and wherein the polypeptide sequence of said engineered cytochrome P450-BM3 variant comprises at least one substitution or substitution set at one or more positions in said polypeptide sequence selected from 45, 45/111/226/347, 45/853, 110/114, 111, 111/127/226/244, 111/194/244/347/853, 111/194/347, 111/194/347/853, 111/210, 111/210/347/853, 111/226, 111/226/244/347, 111/226/853/969, 111/244, 111/244/853, 111/347/853, 111/853, 114, 114/245, 127/210/244, 127/210/244/853, 127/210/347/853, 127/244, 127/347, 194/853, 210/853, 226/236/244/347/853, 236/244, 237, 237/245, 244/853, 245, 518, 518/652, 519, 562, 563, 584/724, 586, 616, 618, 619, 621, 623, 628, 640, 653, and 666 wherein the amino acid positions of said polypeptide sequence are numbered with reference to SEQ ID NO: 984.

18. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 1160, and wherein the polypeptide sequence of said engineered cytochrome P450-BM3 variant comprises at least one substitution or substitution set at one or more positions in said polypeptide sequence selected from 132/366/433/463/467/793, 132/366/467/661, 132/467/468/506/793, 132/468/793, 132/1025, 183/1025/1045/1048, 290/366/433/463/467, 290/433/467/793/1025, 290/433/793, 290/793, 290/1025, 366/433, 433, 433/467/1025, 433/506/1025, 433/790, 463/793, 473, 793, and 1025, wherein the amino acid positions of said polypeptide sequence are numbered with reference to SEQ ID NO: 1160.

19. The engineered cytochrome P450-BM3 variant of claim 1, wherein said polypeptide sequence has at least 85%, 86%, 87%, 88%, 89%, 90%, 91%, 92%, 93%, 94%, 95%, 96%, 97%, 98%, 99%, or more sequence identity to SEQ ID NO: 1266, and wherein the polypeptide sequence of said engineered cytochrome P450-BM3 variant comprises at least one substitution or substitution set at one or more positions in said polypeptide sequence selected from 114/230/446/853, 114/292/293/296/463/853, 132/183/366/467/661/1025/1045/1048, 132/290/366/433/467/661/793/1025, 132/290/366/467/661/793, 132/290/366/467/661/1025, 132/366/433/467/506/661/1025, 132/366/433/467/661, 132/366/433/467/661/790, 132/366/463/467/661/793, 132/366/467/473/661, 132/366/467/661/793, 366/467/468/506/661/793, 433/463/467/661/793, 463, 463/853, 689, 720, 724, 730, 769, 780, 792, 810, 817, 824, 853, 923, 926, 939, 952, 962, 968, 974, 979, 981, 995, 1006, 1015, 1017, 1022, 1027, 1031, 1040, 458, 518/653, 519/628, 616/619, and 653 wherein the amino acid positions of said polypeptide sequence are numbered with reference to SEQ ID NO: 1266.

20. The engineered cytochrome P450-BM3 variant of claim 1, wherein said engineered cytochrome P450-BM3 variant comprises a polypeptide sequence selected from the group consisting of (a) a polypeptide sequence that is at least 85%, 86%, 87%, 88%, 89%, 90%, 91%, 92%, 93%, 94%, 95%, 96%, 97%, 98%, 99% or more identical to SEQ ID NO: 4, 36, 66, 72, 198, 226, 244, 286, 358, 410, 534, 734, 748, 828, 968, 984, 1160, or 1266;
(b) a polypeptide sequence set forth in SEQ ID NO: 36, 66, 72, 198, 226, 244, 286, 358, 410, 534, 734, 748, 828, 968, 984, 1160, or 1266; and
(c) a polypeptide sequence forth in at least one of the even numbered sequences of SEQ ID NOS: 4-1368.

21. The engineered cytochrome P450-BM3 variant of claim 1, wherein said engineered cytochrome P450-BM3 variant comprises at least one improved property, as compared to a wild-type Bacillus megaterium cytochrome P450-BM3 or engineered P450-BM3 variant.

22. The engineered cytochrome P450-BM3 variant of claim 21, wherein said improved property comprises a property selected from the properties consisting of (a) an improved activity on a substrate, (b) improved thermostability, (c) increased activity on a substrate after preincubation at 42.5° C. and (d) improved stereoselectivity toward one or more diastereomer products.

23. The engineered cytochrome P450-BM3 variant of claim 22, wherein said substrate comprises 1-tert-Butoxycarbonylaminocyclopentanoic acid (Compound (1)).

24. A polynucleotide sequence encoding at least one engineered cytochrome P450-BM3 variant of claim 20, said polynucleotide sequence comprises at least 85%, 86%, 87%, 88%, 89%, 90%, 91%, 92%, 93%, 94%, 95%, 96%, 97%, 98%, 99%, or more sequence identity to SEQ ID NOS: 3, 35, 65, 71, 197, 225, 243, 285, 357, 409, 533, 733, 747, 827, 967, 983, 1159, or 1265, wherein the polynucleotide sequence of said engineered cytochrome P450-BM3 variant comprises at least one substitution at one or more positions.

25. An expression vector comprising at least one polynucleotide sequence of claim 24.

26. A host cell comprising at least one polynucleotide sequence of claim 24.

27. A method of producing an engineered cytochrome P450-BM3 variant in a host cell, comprising culturing a host cell of claim 26, under suitable conditions, such that at least one engineered cytochrome P450-BM3 variant is produced.
